(19) United States
(12) Patent Application Publication
(10) Pub. No.: US 2024/0408408 A1
(43) Pub. Date: Dec. 12, 2024

(54) FAP-TARGETED NEUTRON CAPTURE AGENTS, AND USES AND FORMULATIONS RELATED THERETO

(71) Applicant: Trustees of Tufts College, Medford, MA (US)

(72) Inventors: William W. Bachovchin, Cambridge, MA (US); Hung-sen Lai, Andover, MA (US); Wengen Wu, Winchester, MA (US)

(21) Appl. No.: 18/701,061

(22) PCT Filed: Oct. 21, 2022
(86) PCT No.: PCT/US22/47424

§ 371 Date: Apr. 12, 2024

Related U.S. Application Data
(60) Provisional application No. 63/270,747, filed on Oct. 22, 2021.

(51) Int. Cl. A61N 5/10; A61K 41/00; A61K 47/64; C07F 5/02

(52) U.S. Cl. CPC A61N 5/10; A61K 41/0095; A61K 47/64; C07F 5/025; A61N 2005/109

(57) ABSTRACT

Disclosed are compositions for delivering neutron capture agents to cells expressing fibroblast activation protein (“FAP”), which composition comprises an FAP binding moiety associated (covalently or non-covalently) with a neutron capture agent.

### CROSS-REFERENCES TO RELATED APPLICATIONS

This application claims the benefit of priority to U.S. Provisional Application No. 63/270,747, filed Oct. 22, 2021; which is incorporated herein by reference in its entirety.

### BACKGROUND

Fibroblast-activation protein (FAP), also known as seprase, is a type II integral membrane serine peptidase. FAP belongs to the dipeptidyl peptidase IV family. It is a 170 kDa homodimer containing two N-glycosylated subunits with a large C-terminal extracellular domain, in which the enzyme's catalytic domain is located. FAP, in its glycosylated form, has both post-prolyl dipeptidyl peptidase and gelatinase activities. Homologues of human FAP were found in several species, including mice and cynomolgus monkeys.

FAP is expressed selectively in reactive stromal fibroblasts of more than 90% of epithelial malignancies (primary and metastatic) examined, including lung, colorectal, bladder, ovarian and breast carcinomas, and in malignant mesenchymal cells of bone and soft tissue sarcomas, while it is generally absent from normal adult tissues (Brennen et al., Mol. Cancer Ther. 11 (2); 257-266 (2012); Garin-Chesa et al., Proc Natl Acad Sci USA 87, 7235-7239 (1990); Rettig et al., Cancer Res. 53:3327-3335 (1993); Rettig et al., Proc Natl Acad Sci USA 85, 3110-3114 (1988)). FAP is also expressed on certain malignant tumor cells.

Due to its expression in many common cancers and its restricted expression in normal tissues, FAP is a promising antigenic target for imaging, diagnosis, and therapy of a variety of cancers. Accordingly, there is an ongoing need for therapies that target FAP.

### SUMMARY OF THE INVENTION

The present invention provides composition for delivering neutron capture agents to cells expressing fibroblast activation protein (“FAP”), which composition comprises an FAP binding moiety (an “FAP-bm”) associated (covalently or non-covalently) with a neutron capture agent, wherein binding of the FAP-bm to FAP on the surface of a cell expressing FAP results in FAP-dependent localization of the neutron capture agent to tissue expressing FAP. Optionally, binding of the FAP-bm to FAP also causes intracellular internalization of the neutron capture agent such that the neutron capture agent is selectively taken up by cells expressing FAP in a manner dependent on the FAP-bm.

In certain embodiments, the neutron capture agent comprises the Boron isotope ¹⁰B.

In certain embodiments, the neutron capture agent comprises the Lithium isotope ⁶Li.

In certain embodiments, the neutron capture agent comprises the He isotope ³He.

In certain embodiments, the neutron capture agent comprises the Cadmium isotope ¹³Cd.

In certain embodiments, the neutron capture agent comprises the Samarium isotope ¹⁴⁹Sm.

In certain embodiments, the neutron capture agent comprises the Gadolinium isotope ¹⁵⁷Gd.

In certain embodiments, the neutron capture agent comprises Au (Gold), such as ¹⁹⁷Au.

In certain embodiments, the neutron capture agent comprises Hf (Hafnium), such as ¹⁷⁴Hf, ¹⁷⁶Hf, ¹⁷⁷Hf, ¹⁷⁸Hf, ¹⁷⁹Hf or ¹⁸⁹Hf, preferably ¹⁷⁷Hf or ¹⁷⁹Hf.

In certain embodiments, the neutron capture agent comprises an isotope with a favorable neutron capture cross section.

To further illustrate, in certain aspects the present disclosure provides particles, such as nanoparticles, liposomes. PEG or the like, comprising a neutron capture agent to be delivered to a cell expressing FAP, and having appended to the particle one or more FAP targeting moieties having a structure represented by formula II:

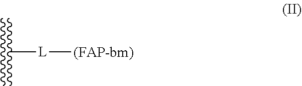

wherein.
FAP-bm is FAPi or FAPb;
FAPi is a compound that covalently binds to FAP;
FAPb is a compound that non-covalently binds to FAP; and
L is bind or a linker.

To further illustrate, in certain aspects the present disclosure provides one or more carborane(s) including one or more ¹⁰B atoms having associated therewith (covalent or non-covalently) one or more FAP targeting moieties having a structure represented by formula II:

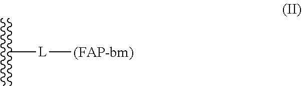

wherein,
FAP-bm is FAPi or FAPb;
FAPi is a compound that covalently binds to FAP;
FAPb is a compound that non-covalently binds to FAP; and
L is bind or a linker.

To further illustrate, in certain aspects the present disclosure provides ¹⁵⁷Gd-chelates or ¹⁵⁷Gd oxide nanoparticles having associated therewith (covalently or non-covalently) one or more FAP targeting moieties having a structure represented by formula II:

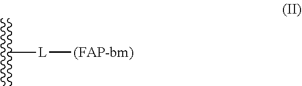

wherein,
FAP-bm is FAPi or FAPb;
FAPi is a compound that covalently binds to FAP;
FAPb is a compound that non-covalently binds to FAP; and
L is bind or a linker.

In certain embodiments, the present disclosure provides gold particles, such as gold nanoparticles comprising ¹⁹⁷Au as a neutron capture agent, and having appended to the particle one or more FAP targeting moieties having a structure represented by formula II:

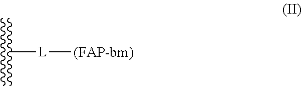

wherein.
FAP-bm is FAPi or FAPb;
FAPi is a compound that covalently binds to FAP;
FAPb is a compound that non-covalently binds to FAP; and
L is bind or a linker.

In a further embodiment, the invention comprises methods of concentrating neutrons in a cell comprising (i) administering the FAP-targeted neutron capture agent to a patient, and (ii) irradiating the patient with neutrons.

In certain embodiments, the present invention provides a compound of formula I:

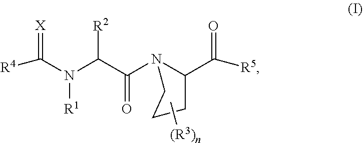

or a pharmaceutically acceptable salt thereof, wherein:
X is O or S;
R¹ is H or (C₁-C₆) alkyl;
R² is H or (C₁-C₆) alkyl;
R³ is halogen, nitro, cyano, amino, acylamino, amido, hydroxyl, alkoxy, acyloxy, thiol, alkylthio, alkyl, aralkyl, heteroaralkyl, alkenyl, alkynyl, carbocyclyl, heterocyclyl, aryl, or heteroaryl;
n is an integer selected from 0 to 7;
R⁴ is amino, alkoxy, acyloxy, alkyl, aralkyl, heteroaralkyl, alkenyl, alkynyl, carbocyclyl, heterocyclyl, aryl, or heteroaryl; and
R⁵ is a moiety comprising at least one boron atom.

In certain embodiments, the present invention provides a compound of formula II:

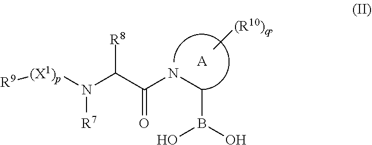

or a pharmaceutically acceptable salt thereof, wherein
A is a 4- to 7-membered heterocycle;
R⁷ is H or (C₁-C₆) alkyl;
R⁸ is H or (C₁-C₆) alkyl;
R¹⁰ is halogen, nitro, cyano, amino, amido, hydroxyl, alkoxy, aryloxy, acyloxy, carboxyl, thiol, alkylthio, arylthio, acylthio, alkyl, alkenyl, alkynyl, carbocyclyl, heterocyclyl, aryl, or heteroaryl;
q is an integer selected from 0 to 10, as valency permits;
X¹ is an alpha amino acid residue, or —X^{1A}—X^{1B}—X^{1C}—X^{1D}— wherein each X^{1A}, X^{1B}, X^{1C} and X^{1D} is independently a bond, —C(O)—, —CH₂C(O)—, alpha amino acid residue, substituted or unsubstituted (C₁-C₁₂) alkylene, substituted or unsubstituted 2 to 12 membered heteroalkylene, substituted or unsubstituted (C₃-C₈) cycloalkylene, substituted or unsubstituted 5 to 8 membered heterocycloalkylene, substituted or unsubstituted (C₆-C₈) arylene, or substituted or unsubstituted 5 to 8 membered heteroarylene, provided that at least one of X^{1A}, X^{1B}, X^{1C} and X^{1D} is not a bond;
p is an integer selected from 0 to 5; and
R⁹ is a moiety comprising at least one boron atom.

In certain embodiments, the present invention provides a pharmaceutical composition comprising a compound or composition of the invention.

In certain embodiments, the present invention provides a method of treating cancer, comprising administering to a patient in need thereof an effective amount of a compound or composition of the invention.

### BRIEF DESCRIPTION OF THE DRAWINGS

FIG. 1: Schematic representation of a particle loaded with a neutron capture agent (in the illustrated example, o-carborane is shown) and decorated with FAPi or FAPb moieties which direct the FAP-dependent endocytosis of the particle into a cells expressing FAP. Exemplary particles include liposomes. FAP-bm-decorated neutron capture agents carriers bind specifically to cell surface expressed FAP, which is overexpressed in tumor stromal cells and on cancer cells, which leads to site-specific delivery of FAP-bm-conjugated particle and increases in the concentration of neutron capture agents at the target site. FAP-bm-based delivery vehicles get internalized by target cells through FAP-mediated endocytosis.

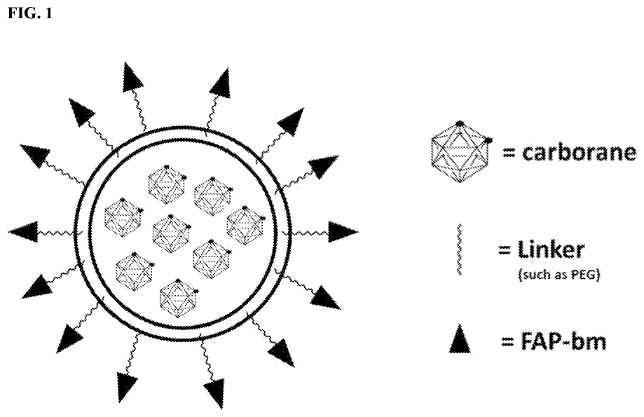

FIG. 2: Exemplary FAP inhibitor-mediated lipid/liposomal based delivery systems. (a) Common lipids used in drug delivery. Neutral lipids include cholesterol and cationic lipids, including DODAC, DSPC, DSPE, DOPE, DOTAP, and PEG. (b) Lipid/liposome nanoparticle conjugated to a FAP inhibitor (d-Ala-boroPro shown by way of example) through a PEG linker. DODAC, N—N-dioleoyl-N,N-dimethyl ammonium chloride; DOTAP, 1,2-dioleoyl-3-trimethylammoniumpropane; DSPC, 1,2-distearoyl-sn-glycero-3-phosphocholine; DSPE, 1,2-disteroylphosphatidyl ethanolamine; FR, folate receptor; PEG, polyethylene glycol. Liposomes/lipoplexes have proved to be efficient delivery agents for the transport of chemotherapeutic agents as well as neutron capture agent-based therapeutic agents. Liposomes are the vesicular structure, consisting of an amphiphilic phospholipid bilayer. This bilayer generally allows the encapsulation of the neutron capture agents (chelated 157-Gallodium complexes shown) into the central aqueous space of the liposome. Lipid-based nanoparticles are associated with several advantages such as low toxicity, biocompatibility, ease of production, and the ability to encapsulate both hydrophobic and hydrophilic neutron capture agents. The use of a FAP-bm, such as a FAP inhibitor, as a targeting ligand can be carried out to create surface modification of liposomes to ensure tumor-specific delivery of neutron capture agents and result in enhanced cellular uptake and improved biodistribution in the targeted FAP-expressing tissue.

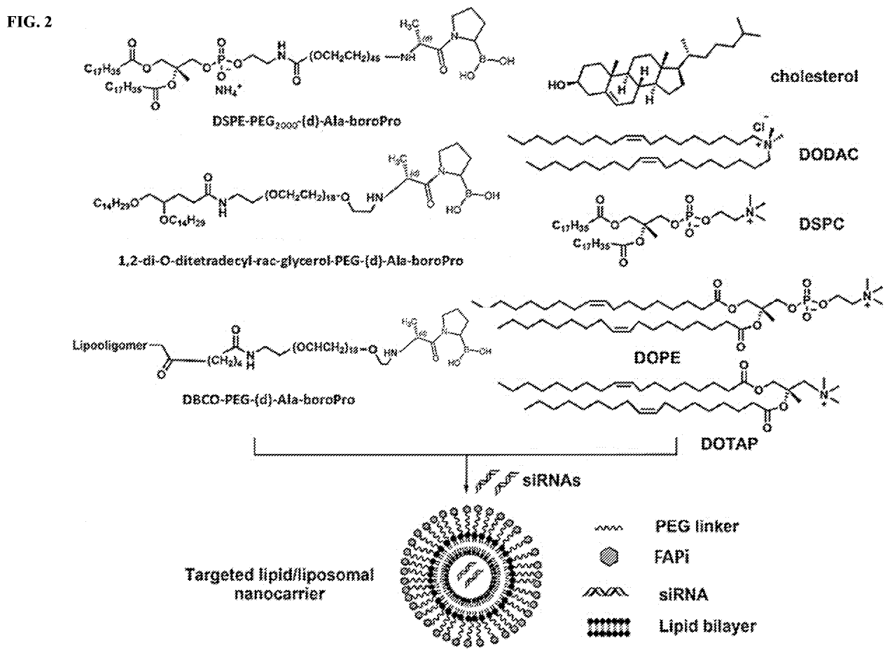

FIG. 3: Illustrates on embodiment of a FAPi-mediated chitosan-based nanoparticle delivery system. (a) a FAPi moiety is conjugated to chitosan, OCMS, DEAE. (b) FAPi-conjugated chitosan nanoparticles. DEAE, diethylaminoethyl; OCMS, O-carboxymethyichitosan.

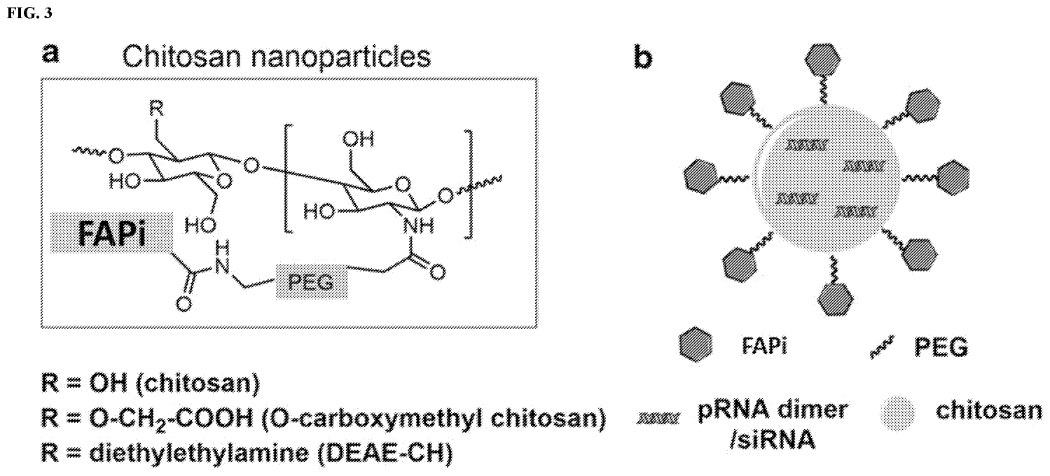

### DETAILED DESCRIPTION OF THE INVENTION

The tumor stroma, which accounts for a large part of the tumor mass. Among the stroma, is a subpopulation of cells known as cancer-associated fibroblasts (CAFs), which are present in more than 90% of epithelial carcinomas, including pancreatic, colon, and breast cancer. Cancer-associated fibroblasts feature high expression of FAP, which is not detectable in adult normal tissue but is associated with a poor prognosis in cancer patients. Thus. CAFs and FAP represent an attractive target for the delivery of diagnostic and therapeutic compounds.

The present invention provides composition for delivering neutron capture agents to cells expressing fibroblast activation protein (“FAP”), which composition comprises an FAP binding moiety (an “FAP-bm”) associated (covalently or non-covalently) with a neutron capture agent, wherein binding of the FAP-bm to FAP on the surface of a cell expressing FAP results in intracellular internalization of the FAP-bm, and with it the neutron capture agent.

In certain embodiments, FAP-bm is FAPi. In certain embodiments, FAPi covalently binds to a side chain of an amino acid in an active site of FAP, such as the active site serine. In certain embodiments, the covalent bond between FAPi and FAP is reversible. In certain embodiments, the covalent bond between FAPi and FAP is irreversible. In certain embodiments, FAPi comprises a moiety which has a Kᵢ for FAP that is at least 10× smaller as compared to the Kᵢ of prolyl endopeptidase for FAP (EC 3.4.21.26; PREP). In certain embodiments, FAPi comprises a moiety which has a Kᵢ for FAP that is at least 100× smaller as compared to the Kᵢ of prolyl endopeptidase for FAP (EC 3.4.21.26; PREP). In certain embodiments, FAPi comprises a moiety which has a Kᵢ for FAP that is at least 1.000× smaller as compared to the Kᵢ of prolyl endopeptidase for FAP (EC 3.4.21.26; PREP). In certain embodiments, FAPi comprises a moiety which has a Kᵢ for FAP that is at least 5,000× smaller as compared to the Kᵢ of prolyl endopeptidase for FAP (EC 3.4.21.26; PREP). In certain embodiments, FAPi comprises a moiety which has a Kᵢ for FAP that is at least 10,000× smaller as compared to the Kᵢ of prolyl endopeptidase for FAP (EC 3.4.21.26; PREP). In certain embodiments, FAPi comprises a moiety which has a Kᵢ for FAP that is less than 10⁻⁶M. In certain embodiments, FAPi comprises a moiety which has a Kᵢ for FAP that is less than 10⁻⁷M. In certain embodiments, FAPi comprises a moiety which has a Kᵢ for FAP that is less than 10⁻⁸M. In certain embodiments, FAPi comprises a moiety which has a Kᵢ for FAP that is less than 10⁻⁹M. In certain embodiments, FAPi comprises a moiety which has a Kᵢ for FAP that is less than 10⁻¹⁰M.

In certain embodiments, FAP-bm is FAPb. In certain embodiments, FAP_{b} forms a complex with FAP. In certain embodiments, FAP_{b} comprises a moiety which has a K_{d} for FAP that is less than 10⁻⁶M. In certain embodiments, FAP_{b} comprises a moiety which has a K_{d} for FAP that is less than 10⁻⁷M. In certain embodiments, FAP_{b} comprises a moiety which has a K_{d} for FAP that is less than 10⁻⁸M. In certain embodiments. FAP_{b} comprises a moiety which has a K_{d} for FAP that is less than 10⁻⁹M. In certain embodiments, FAP_{b} comprises a moiety which has a K_{d} for FAP that is less than 10⁻¹⁰M.

In certain embodiments, the FAPi moiety comprises a structure represented by formula IIIa:

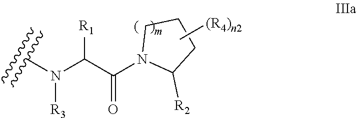

wherein,
R₁ is H or alkyl;
R₂ is a moiety which covalently binds to a side chain of an amino acid in an active site of FAP;
R₃ is H or alkyl;
R₄, independently for each occurrence, is an alkyl, hydroxyl, amino, or halo;
m is 1-3; and
n₂ is 0-7.

In certain embodiments, the FAPi moiety comprises a structure represented by formula IIIb:

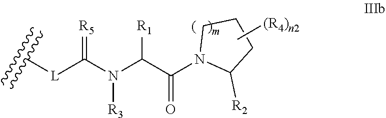

wherein,
L is a bond or a covalent linker;
R₁ is H or alkyl;
R₂ is a moiety which covalently binds to a side chain of an amino acid in an active site of FAP;
R₃ is H or alkyl;
R₄ is alkyl, hydroxyl, amino, or halo;
R₅ is O or S;
m is 1-3; and
n₂ is 0-7.

In certain embodiments, the FAPi moiety comprises a structure represented by formula IVa or a pharmaceutically acceptable salt thereof:

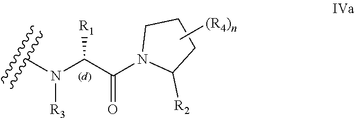

wherein,
R₁ is H or alkyl;
R₂ is a moiety which covalently binds to a side chain of an amino acid in an active site of FAP;
R₃ is H or alkyl;
R₄ is, independently for each occurrence, alkyl, hydroxyl, amino, or halo; and
n is 0-7.

In certain embodiments, the FAPi moiety comprises a structure represented by formula IVb or a pharmaceutically acceptable salt thereof:

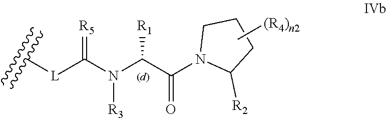

wherein,
L is a bond or a covalent linker;
R₁ is H or alkyl;
R₂ is a moiety which covalently binds to a side chain of an amino acid in an active site of FAP;
R₃ is H or alkyl;
R₄ is, independently for each occurrence, alkyl, hydroxyl, amino, or halo;
R₅ is 0 or S; and
n is 0-7.

In certain embodiments of structures IIIa, IIIb, IVa and IVb above, R₁ is (C₁-C₆)alkyl (e.g., methyl or ethyl).

In certain embodiments of structures IIIa, IIIb, IVa and IVb above, R₂ is B(Y¹)(Y²), CN, or formyl; wherein Y¹ and Y² are each hydroxyl; or Y¹ and Y² together with the boron atom to which they are attached combine to form a moiety which is hydrolysable to a boronic acid. In certain embodiments, Y¹ and Y² together with the boron atom to which they are attached combine to form a 5- to 8-membered ring. In certain embodiments, R₂ is B(OH)₂.

In certain embodiments of structures IIIa, IIIb, IVa and IVb above, R₃ is (C₁-C₆)alkyl. In certain embodiments, R₃ is H.

In certain embodiments of structures IIIa, IIIb, IVa and IVb above, R₄ is (C₁-C₆)alkyl.

In certain embodiments, R₄ is halo (e.g., fluorine).

In certain embodiments of structures IIIb and IVb above, R₅ is O.

In certain embodiments, n or n₂ is 2; and R₄ is fluorine.

In certain embodiments, n or n₂ is 0.

In certain embodiments, n is 1.

In certain embodiments, the FAPi moiety comprises a structure represented by formula V or a pharmaceutically acceptable salt thereof:

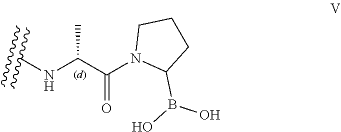

In certain embodiments, the FAPi moiety comprises a structure represented by formula VI or a pharmaceutically acceptable salt thereof:

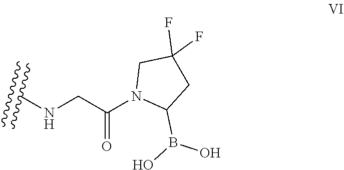

In certain embodiments, the linker L is a bivalent linker covalently connects the FAP-bm to an atom of the neutron capture agent to be delivered. The linker may have a wide variety of lengths, such as in the range from about 2 to about 100 atoms. The atoms used in forming the linker may be combined in all chemically relevant ways, such as chains of carbon atoms forming alkylene, alkenylene, and alkynylene groups, and the like; chains of carbon and oxygen atoms forming ethers, polyoxyalkylene groups, or when combined with carbonyl groups forming esters and carbonates, and the like; chains of carbon and nitrogen atoms forming amines, imines, polyamines, hydrazines, hydrazones, or when combined with carbonyl groups forming amides, ureas, semicarbazides, carbazides, and the like; chains of carbon, nitrogen, and oxygen atoms forming alkoxyamines, alkoxylamines, or when combined with carbonyl groups forming urethanes, amino acids, acyloxylamines, hydroxamic acids, and the like; and others. In addition, it is to be understood that the atoms forming the chain in each of the foregoing illustrative embodiments may be either saturated or unsaturated, such that for example, alkanes, alkenes, alkynes, imines, and the like may be radicals that are included in the linker. In addition, it is to be understood that the atoms forming the linker may also be cyclized upon each other to form divalent cyclic structures that form the linker, including cycloalkanes, cyclic ethers, cyclic amines, arylenes, heteroarylenes, and the like in the linker.

In another embodiment, the linker includes radicals that form at least one releasable linker, and optionally one or more spacer linkers. As used herein, the term releasable linker refers to a linker that includes at least one bond that can be broken under physiological conditions, such as a pH-labile, acid-labile, base-labile, oxidatively labile, metabolically labile, biochemically labile, or enzyme-labile bond. It is appreciated that such physiological conditions resulting in bond breaking do not necessarily include a biological or metabolic process, and instead may include a standard chemical reaction, such as a hydrolysis reaction, for example, at physiological pH, or as a result of compartmentalization into a cellular organelle such as an endosome having a lower pH than cytosolic pH.

### FAP-Targeted Particles

In certain embodiments, the neutron capture agents is associated with a particle, preferably a nanoparticle to which the FAP-bm is associated and able to bring about binding (and preferably internalization) of the particle into cells expressing FAP. The neutron capture agents can be contained within the particle (such as a liposome or hollow nanoparticle), be a part of the matrix making up the particle, or associated with the surface of the particle.

To overcome significant barriers in systemic delivery of neutron capture agents, various organic, inorganic, and polymeric material-based nanoparticle delivery systems have developed for the effective delivery of neutron capture agents to the target organ and can be adapted by derivation with FAPi and FAPb moieties to provide for the FAP-targeted delivery of neutron capture agents of the present invention. See, for example, Draz et al. (2014). Nanoparticle-mediated systemic delivery of siRNA for treatment of cancers and viral infections. Theranostics 4:872-892; Babu et al. (2016). Nanoparticles for siRNA based gene silencing in tumor therapy. IEEE Trans Nanobioscience 15:849-863; Gary et al. (2007). Polymer-based siRNA delivery: perspectives on the fundamental and phenomenological distinctions from polymer-based DNA delivery. J Control Release 121:64-73; Li et al. (2008). Tumor-targeted delivery of siRNA by self-assembled nanoparticles. Mol Ther 16:163-169; and Palanca-Wessels et al. (2016). Antibody targeting facilitates effective intratumoral siRNA nanoparticle delivery to HER2-overexpressing cancer cells. Oncotarget 7:9561-9575, all of which are incorporated by reference herein. The small size, large surface area, high loading capacity of neutron capture agents, easy surface functionalization, and increased stability of nanoparticle formulations make polymer-based nanoparticles the efficient delivery platform for neutron capture agents. Polymeric nanoparticles such as chitosan (CS), dendrimers, cyclodextrin, and nanogels have been employed for neutron capture agents delivery and include functional groups amenable to conjugation to FAPi or FAPb moieties. Various synthetic and natural polymers have been developed as the nonviral nanostructures for neutron capture agents delivery, and likewise can be modified to include FAPi or FAPb moieties for targeting. Natural polymers are considered nontoxic, biocompatible, and biodegradable. Several linear and branched synthetic cationic polymers have been reported, which are easy to manufacture, and possess unique self-assembling property, high stability, and ability to form core shell-shaped micelle structure that have chemistries amenable to chemical modification with FAPi or FAPb moieties.

Mesoporous silica nanoparticles (MSN) have been used extensively for various biomedical applications as well as in drug delivery. MSN have proved excellent delivery vehicles for chemotherapeutic drugs and can be readily adapted for delivery of neutron capture agents. It is associated with several advantages, including high surface area, large pore size, low cytotoxicity, sustained delivery, and flexibility in surface modification. See, for example, Pinese et al. (2018).

Graphene oxide (GO) nanoparticles are another delivery platform that can be adapted for FAPi or FAPb-meditated targeting. GO nanoparticles possess beneficial properties to act as a neutron capture agent, including high solubility, flexible surface functionalization properties, high loading capacity, and photothermal effects.

In other embodiments, the invention provides FAPi and FAPb targeted dendrimers for the delivery of neutron capture agents, alone or in combination with other drug moieties. Dendrimers are cationic synthetic polymer-containing highly symmetrical and branched polymeric chains. Dendrimers provide many sites for surface functionalization with FAPi and FAPb moieties, possess vital characteristics such as biocompatibility, provide multivalent surfaces, and have low polydispersity.

In still other embodiments, FAPi and FAPb moieties can be used to functionalize chitosan particles. Chitosan is made from repeating units of N-acetyl glucosamine and glucosamine units having β-1,4 glycosidic linkage. See, for example, Rudzinski et al. (2010). Chitosan as a carrier for targeted delivery of small interfering RNA. Int J Pharm 399:1-11; and Sevilla et al. (2019). Natural polysaccharides for siRNA delivery: nanocarriers based on chitosan, hyaluronic acid, and their derivatives. Molecules 24:2570, both of which are incorporated by reference herein.

There are a wide range of cationic polymers that are amendable to conjugation to a FAPi or FAPb moiety that also be used for the FAP-targeted delivery of neutron capture agents. Exemplary copolymers have the ability to form self-assembled micelle structures and exhibit low cytotoxicity, and are generally regarded as nonimmunogenic nature. These polymer-based delivery platforms have found remarkable results for drug/gene delivery. To illustrate, see Brinkhuis et al. (2011) Polymeric vesicles in biomedical applications. Polym Chem 2:1449-14; Suo et al. (2017). Folate-decorated PEGylated triblock copolymer as a pH/reduction dual-responsive nanovehicle for targeted intracellular co-delivery of doxorubicin and Bcl-2 siRNA. Mater Sci Eng C Mater Biol Appl 76:659-672; Lehner et al. (2017). Efficient receptor mediated siRNA delivery in vitro by folic acid targeted pentablock copolymer-based micelleplexes. Biomacromolecules 18:2654-2662; Wang et al. (2019). Engineering multifunctional bioactive citric acid-based nanovectors for intrinsical targeted tumor imaging and specific siRNA gene delivery in vitro/in vivo. Biomaterials 199:10-21; and Cunningham et al. (2020). Cholic acid-based mixed micelles as siRNA delivery agents for gene therapy. Int J Pharm 578: 119078, all of which are incorporated by reference herein.

### Compounds

In certain embodiments, the present invention provides a compound of formula I:

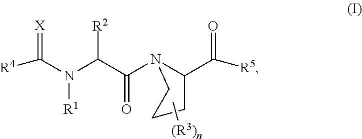

or a pharmaceutically acceptable salt thereof, wherein
X is O or S;
R¹ is H or (C₁-C₆) alkyl;
R² is H or (C₁-C₆) alkyl;
R³ is halogen, nitro, cyano, amino, acylamino, amido, hydroxyl, alkoxy, acyloxy, thiol, alkylthio, alkyl, aralkyl, heteroaralkyl, alkenyl, alkynyl, carbocyclyl, heterocyclyl, aryl, or heteroaryl;
n is an integer selected from 0 to 7;
R⁴ is amino, alkoxy, acyloxy, alkyl, aralkyl, heteroaralkyl, alkenyl, alkynyl, carbocyclyl, heterocyclyl, aryl, or heteroaryl; and
R⁵ is a moiety comprising at least one boron atom.

In certain embodiments, the compound is a compound of formula I-a:

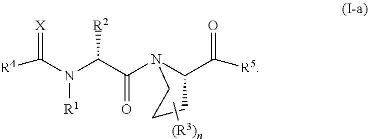

In certain embodiments, X is O.

In certain embodiments, R¹ is H.

In certain embodiments, R² is (C₁-C₆) alkyl. In certain embodiments, R² is methyl.

In certain embodiments, R³ is halogen, nitro, cyano, amino, acylamino, amido, hydroxyl, alkoxy, acyloxy, thiol, or alkylthio. In certain embodiments, R³ is alkyl, aralkyl, heteroaralkyl, alkenyl, alkynyl, carbocyclyl, heterocyclyl, aryl, or heteroaryl.

In certain embodiments, n is 0.

In certain embodiments, R⁴ is amino, alkoxy, or acryloxy. In certain embodiments, R⁴ is arylamino or heteroarylamino. In certain embodiments, R⁴ is pyridinyl.

In certain embodiments, R⁵ is a moiety comprising at least one ¹⁰B. In certain embodiments, R⁵ is a moiety comprising boronophenylalanine, having the structure of

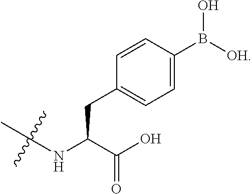 [SMILES: *N[C@@H](Cc1ccc(B(O)O)cc1)C(=O)O]

In certain embodiments, R⁵ is a moiety comprising carborane. In certain embodiments, the carborane is decaborane. In certain embodiments, R⁵ is a moiety further comprising a self-immolative linker. In certain embodiments, the self-immolative linker is selected from the group consisting of:

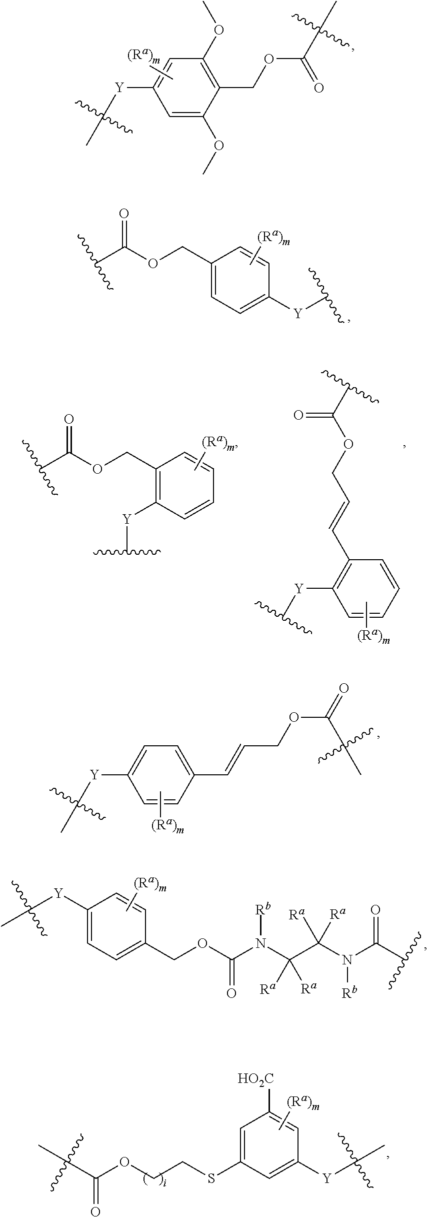

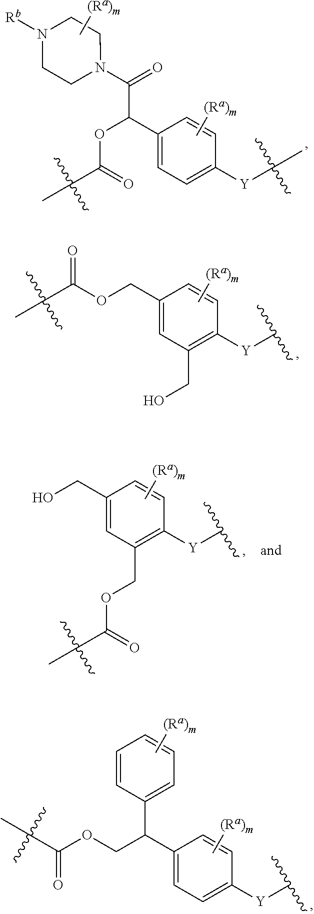

wherein:
each R^{a} independently is halogen, nitro, cyano, amino, amido, hydroxyl, alkoxy, aryloxy, acyloxy, carboxyl, thiol, alkylthio, arylthio, acylthio, alkyl, alkenyl, alkynyl, carbocyclyl, heterocyclyl, aryl, or heteroaryl;
each R^{b} independently is alkyl, aralkyl, heteroaralkyl, alkenyl, alkynyl, carbocyclyl, heterocyclyl, aryl, or heteroaryl;
Y is O, N, or S;
m is an integer selected from 0 to 6, as valency permits; and
i is an integer selected from 1-6.

In certain embodiments, i is 1 or 2. In certain embodiments, the self-immolative linker is

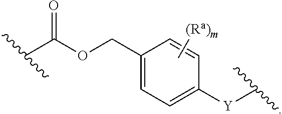

In certain embodiments, Y is N.

In certain embodiments, m is 0.

In certain embodiments, the present invention provides a compound of formula II:

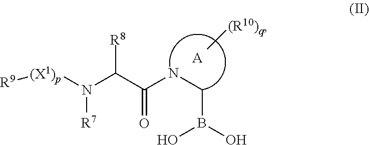

or a pharmaceutically acceptable salt thereof, wherein
A is a 4- to 7-membered heterocycle;
R⁷ is H or (C₁-C₆) alkyl;
R⁸ is H or (C₁-C₆) alkyl;
R¹⁰ is halogen, nitro, cyano, amino, amido, hydroxyl, alkoxy, aryloxy, acyloxy, carboxyl, thiol, alkylthio, arylthio, acylthio, alkyl, alkenyl, alkynyl, carbocyclyl, heterocyclyl, aryl, or heteroaryl;
q is an integer selected from 0 to 10, as valency permits;
X¹ is an alpha amino acid residue, or —X^{1A}—X^{1B}—X^{1C}—X^{1D}— wherein each X^{1A}, X^{1B}, X^{1C} and X^{1D} is independently a bond, —C(O)—, —CH₂C(O)—, alpha amino acid residue, substituted or unsubstituted (C₁-C₁₂) alkylene, substituted or unsubstituted 2 to 12 membered heteroalkylene, substituted or unsubstituted (C₃-C₈) cycloalkylene, substituted or unsubstituted 5 to 8 membered heterocycloalkylene, substituted or unsubstituted (C₆-C₈) arylene, or substituted or unsubstituted 5 to 8 membered heteroarylene, provided that at least one of X^{1A}, X^{1B}, X^{1C} and X1^{D} is not a bond;
p is an integer selected from 0 to 5; and
R⁹ is a moiety comprising at least one boron atom.

In certain embodiments, R⁸ is H or (C₁-C₆) alkyl which is optionally substituted. In certain embodiments, R⁸ is H. In certain embodiments, R⁸ is unsubstituted (C₁-C₆) alkyl. In certain embodiments, R⁸ is a (C₁-C₆) alkyl which is substituted with hydroxyl (—OH).

In certain embodiments, the compound is a compound of formula II-a:

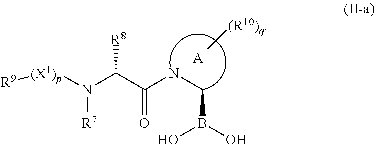

In certain embodiments, the compound is a compound of formula II-b:

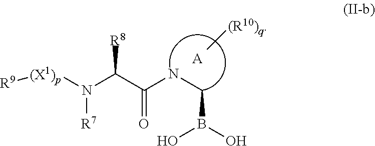

In certain embodiments, A is a 5-membered heterocycle.

In certain embodiments, R⁷ is H.

In certain embodiments, R⁸ is (C₁-C₆) alkyl. In certain embodiments, R⁸ is methyl.

In certain embodiments, q is 0.

In certain embodiments, X¹ includes a natural or modified alpha amino acid residue.

In certain embodiments, the natural alpha amino acid residue is selected from the group consisting of Val, Gly, Ile, Ala, Leu, Met, Phe, Tyr, and Trp. In certain embodiments, the natural alpha amino acid residue is selected from the group consisting of Val, Gly, and Ala.

In certain embodiments, p is an integer selected from 0 to 3. In certain embodiments, each X^{1A} and X^{1D} is independently —CH₂CH₂C(O)—, —CH₂C(O)— or —C(O)—. In certain embodiments, each X^{1B} and X^{1C} is independently a substituted or unsubstituted cyclohexylene, substituted or unsubstituted phenylene, or 2 to 6 membered heteroalkylene. In certain embodiments, each X^{1A} and X^{1D} is independently —CH₂CH₂C(O)—, —CH₂C(O)— or —C(O)—; and each X^{1B} and X^{1C} is independently a substituted or unsubstituted cyclohexylene, substituted or unsubstituted phenylene, or 2 to 6 membered heteroalkylene. In certain embodiments, X^{1A} is —CH₂CH₂C(O)—, —CH₂C(O)— or —C(O)—. In certain embodiments, X^{1B}, X^{1C} and X^{1D} is independently a bond or amino acid residue wherein at least one of X^{1B}, X^{1C} and X^{1D} is not a bond. In certain embodiments, X^{1A} is —CH₂CH₂C(O)—, —CH₂C(O)— or —C(O)—; and X^{1B}, X^{1C} and X^{1D} is independently a bond or amino acid residue wherein at least one of X^{1B}, X^{1C} and X^{1D} is not a bond.

In certain embodiments, X^{1A} is —CH₂CH₂C(O)—. In certain embodiments, X^{1A} is —CH₂C(O)—. In certain embodiments, X^{1A} is —C(O)—. In certain embodiments, X^{1D} is —CH₂CH₂C(O)—. In certain embodiments, X^{1D} is —CH₂C(O)—. In certain embodiments, X^{1D} is —C(O)—. In certain embodiments, X^{1B} is a substituted or unsubstituted cyclohexylene. In certain embodiments, X^{1B} is substituted or unsubstituted phenylene. In certain embodiments, X^{1B} is 2 to 6 membered heteroalkylene. In certain embodiments, X^{1C} is a substituted or unsubstituted cyclohexylene. In certain embodiments, X^{1C} is substituted or unsubstituted phenylene. In certain embodiments, X^{1C} is 2 to 6 membered heteroalkylene.

In certain embodiments, X¹ is

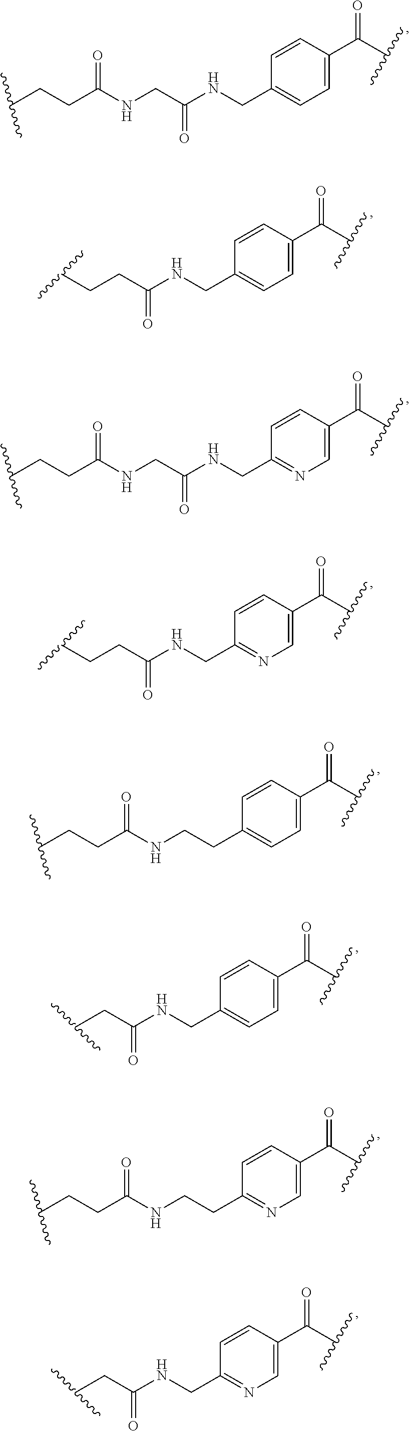

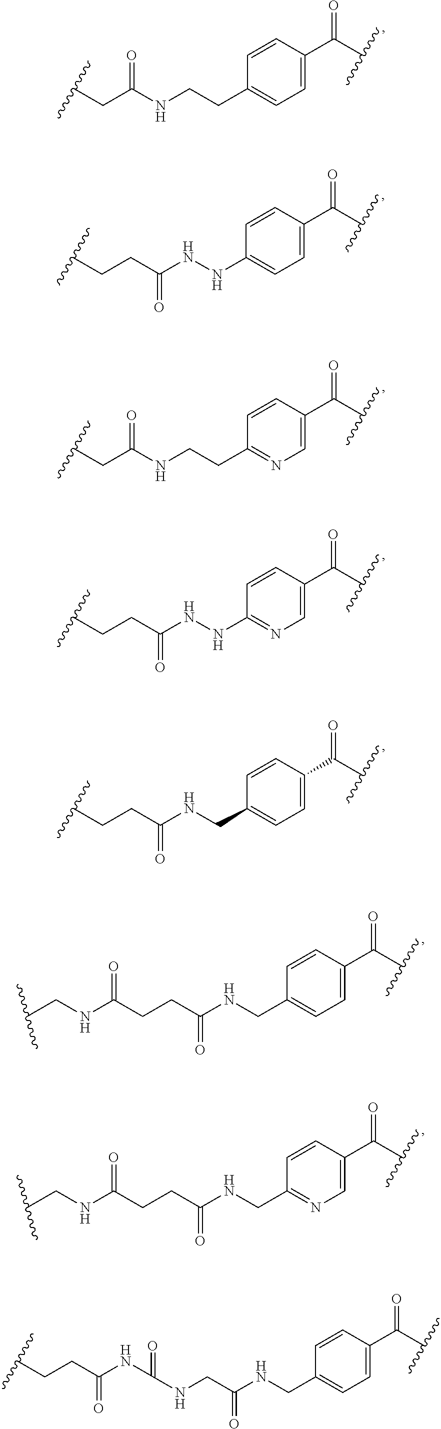

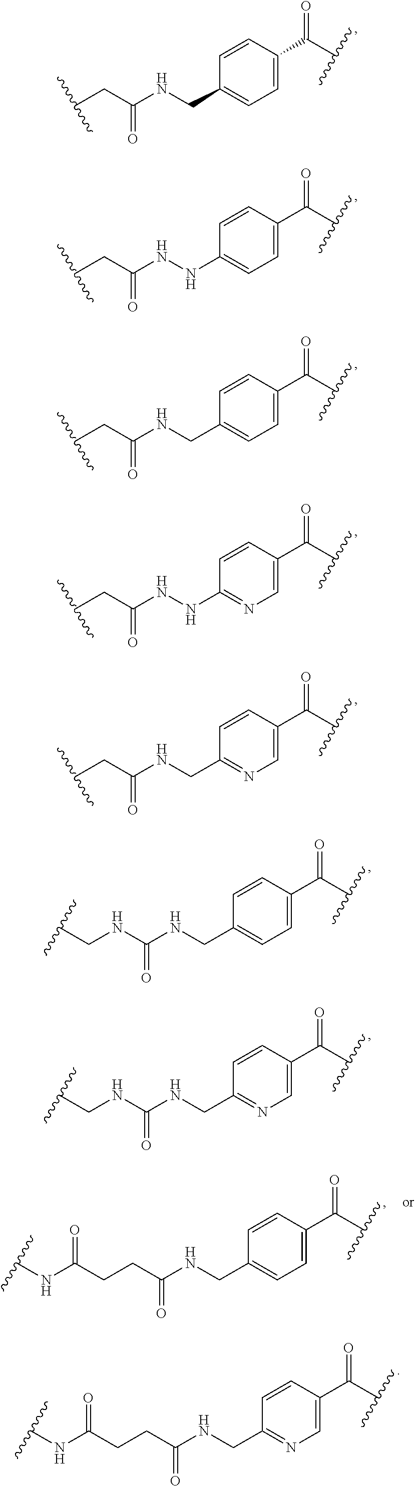

In certain embodiments, X^{1B} is a bond. In certain embodiments, X^{1B} is amino acid residue. In certain embodiments, X^{1C} a bond. In certain embodiments, X^{1C} is amino acid residue. In certain embodiments, X^{1D} is a bond. In certain embodiments, X^{1D} is amino acid residue.

In certain embodiments, X¹ is

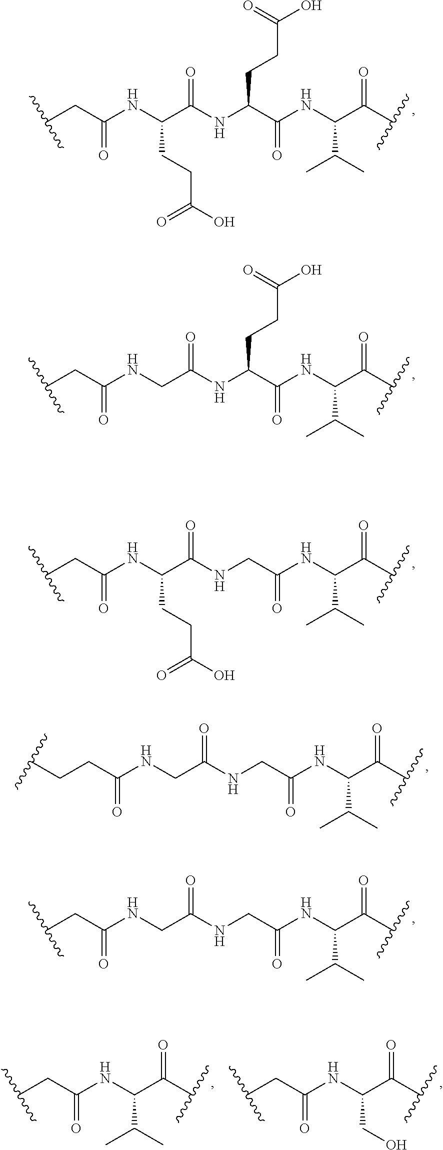

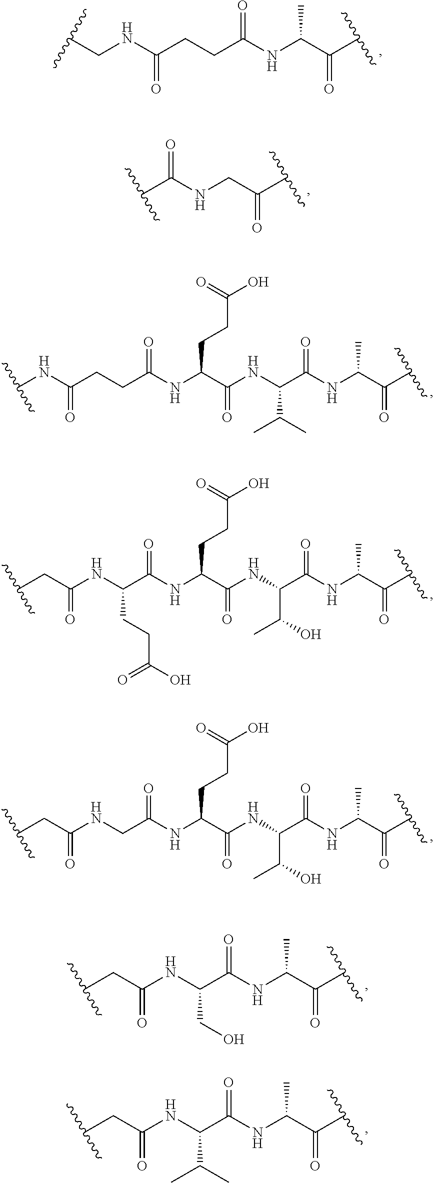

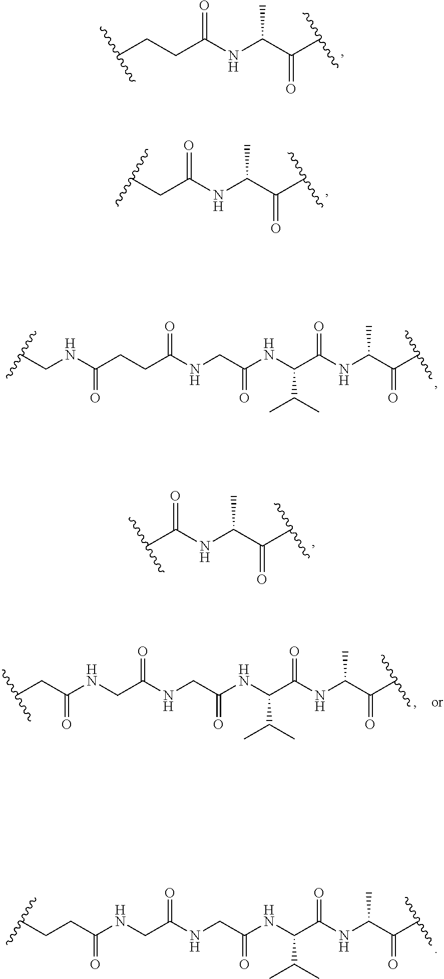

In certain embodiments, X^{1A} is —C(O)— and X^{1D} is unsubstituted C₁-C₄ alkylene. In certain embodiments, X^{1D} is —C(O) and X^{1A} is unsubstituted C₁-C₄ alkylene.

In certain embodiments, X¹ is

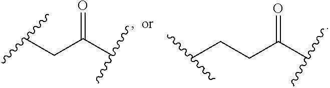 [SMILES: *CC(*)=O ; *CCC(*)=O]

In certain embodiments, R⁹ is a moiety comprising at least one ¹⁰B. In certain embodiments, R⁹ is a moiety comprising carborane. In certain embodiments, the carborane is decaborane.

In certain embodiments, the boron atom of the boronic acid comprises a greater than natural abundance of ¹⁰B. In certain embodiments, the boron atom of the boronic acid comprises at least about 25% ¹⁰B. In certain embodiments, boron atom of the boronic acid comprises at least about 50% ¹⁰B.

Exemplary compounds having Formula (II) or (II-a) may include, but are not limited to:

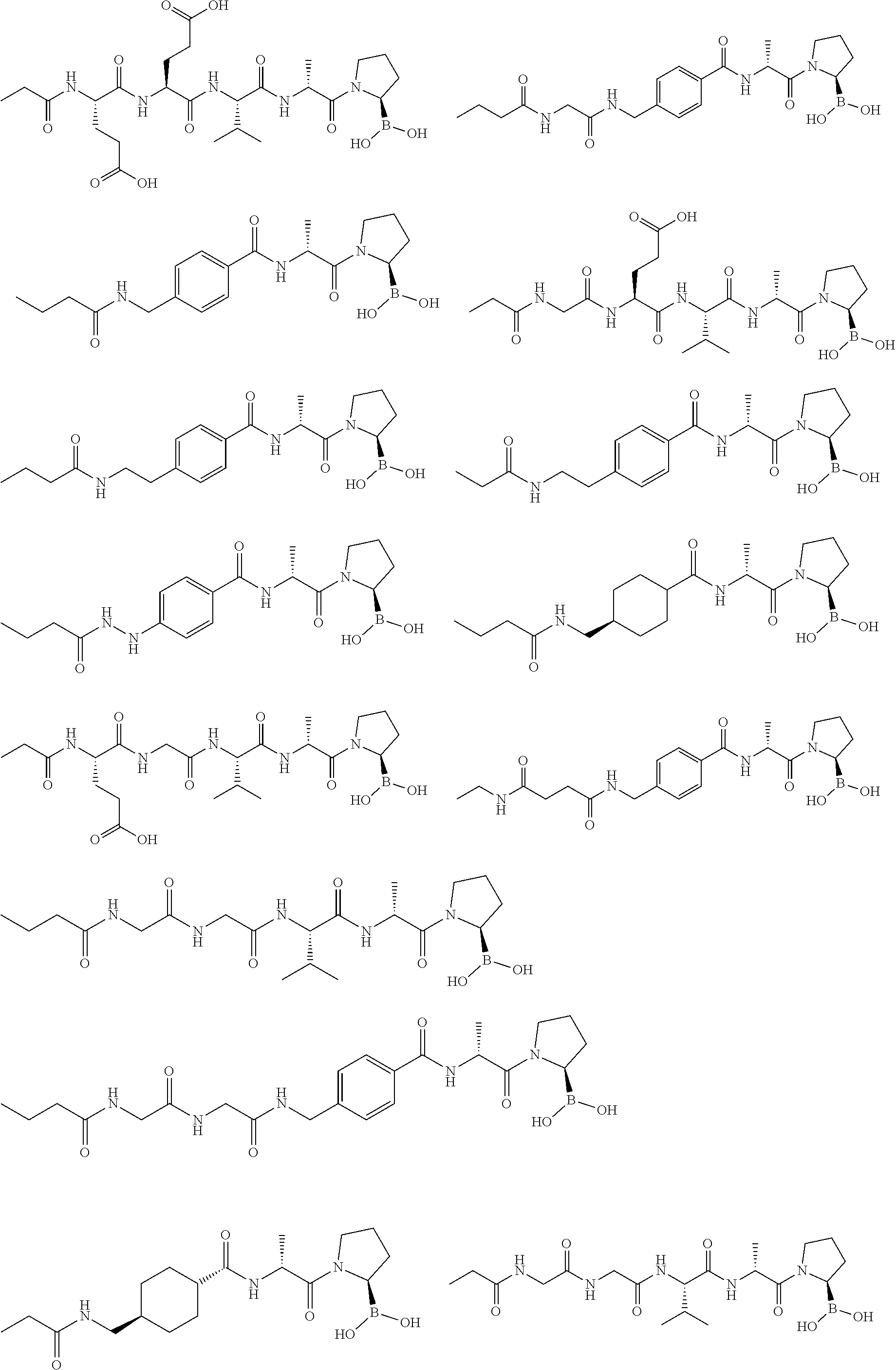

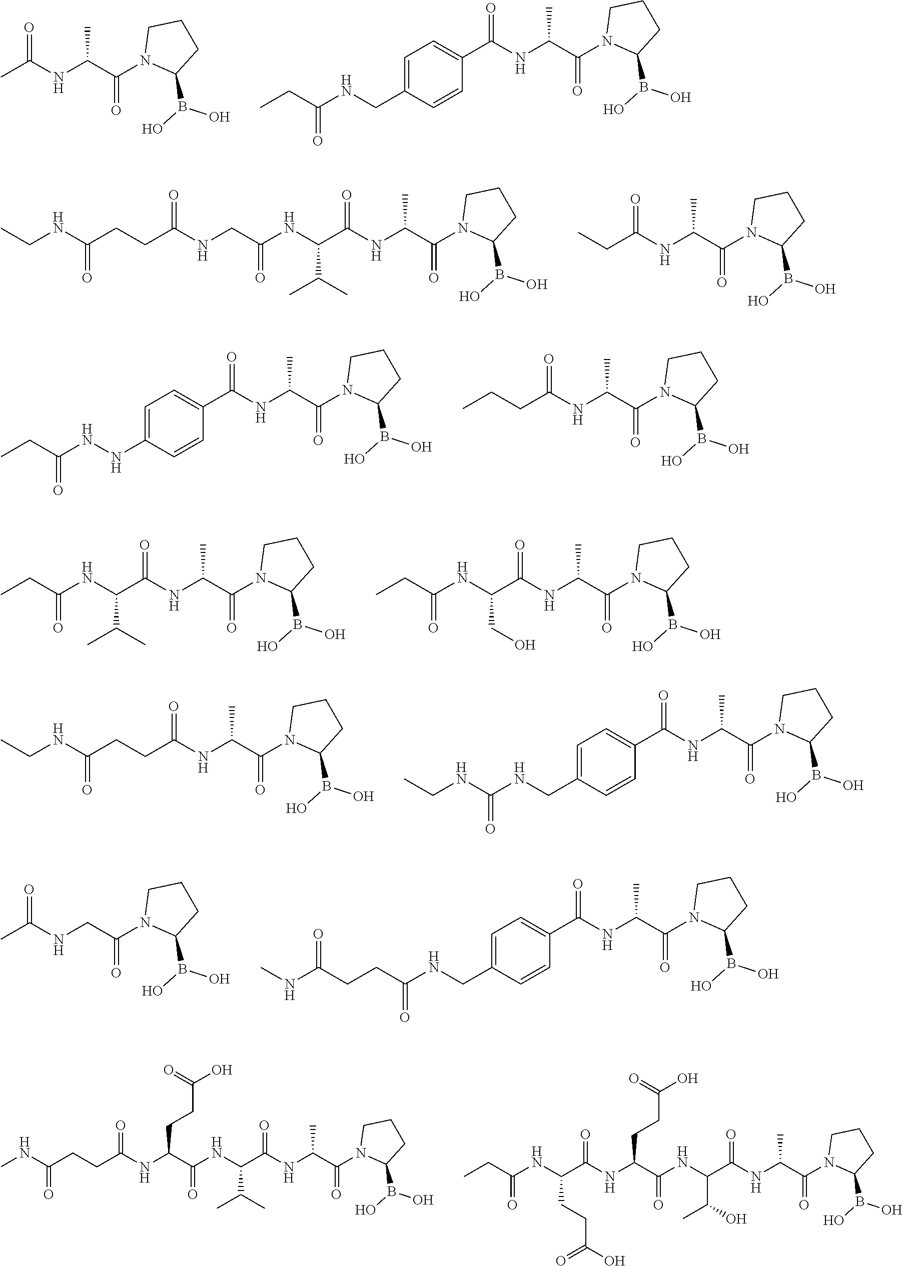

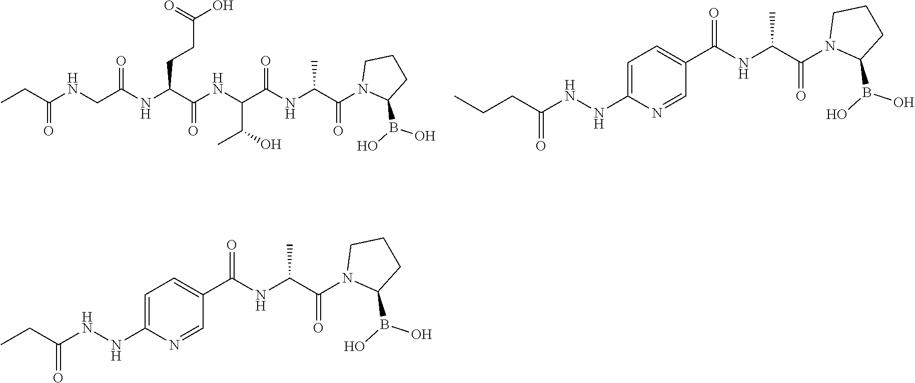 [SMILES: CCC(=O)NCC(=O)N[C@@H](CCC(=O)O)C(=O)NC(C(=O)N[C@H](C)C(=O)N1CCC[C@H]1B(O)O)[C@@H](C)O ; CCCC(=O)NNc1ccc(C(=O)N[C@H](C)C(=O)N2CCC[C@H]2B(O)O)cn1 ; CCC(=O)NNc1ccc(C(=O)N[C@H](C)C(=O)N2CCC[C@H]2B(O)O)cn1]

In certain embodiments, the present invention provides a pharmaceutical composition comprising a compound or composition of the invention.

In certain embodiments, the present invention provides a method of treating cancer, comprising administering to a patient in need thereof a compound or composition of the invention. In certain embodiments, the method comprises irradiating the patient with neutrons. In certain embodiments, the cancer is selected from lung cancer, colorectal cancer, bladder cancer, ovarian cancer, breast cancer, bone cancer and soft tissue sarcoma.

In certain embodiments, the present invention provides a method of concentrating neutrons in a cell comprising (i) administering a compound or composition of the invention to a patient, and (ii) irradiating the patient with neutrons.

### Pharmaceutical Compositions

The compositions and methods of the present invention may be utilized to treat an individual in need thereof. In certain embodiments, the individual is a mammal such as a human, or a non-human mammal. When administered to an animal, such as a human, the composition or the compound is preferably administered as a pharmaceutical composition comprising, for example, a compound of the invention and a pharmaceutically acceptable carrier. Pharmaceutically acceptable carriers are well known in the art and include, for example, aqueous solutions such as water or physiologically buffered saline or other solvents or vehicles such as glycols, glycerol, oils such as olive oil, or injectable organic esters. In preferred embodiments, when such pharmaceutical compositions are for human administration, particularly for invasive routes of administration (i.e., routes, such as injection or implantation, that circumvent transport or diffusion through an epithelial barrier), the aqueous solution is pyrogen-free, or substantially pyrogen-free. The excipients can be chosen, for example, to effect delayed release of an agent or to selectively target one or more cells, tissues or organs. The pharmaceutical composition can be in dosage unit form such as tablet, capsule (including sprinkle capsule and gelatin capsule), granule, lyophile for reconstitution, powder, solution, syrup, suppository, injection or the like. The composition can also be present in a transdermal delivery system. e.g., a skin patch. The composition can also be present in a solution suitable for topical administration, such as a lotion, cream, or ointment.

A pharmaceutically acceptable carrier can contain physiologically acceptable agents that act, for example, to stabilize, increase solubility or to increase the absorption of a compound such as a compound of the invention. Such physiologically acceptable agents include, for example, carbohydrates, such as glucose, sucrose or dextrans, antioxidants, such as ascorbic acid or glutathione, chelating agents, low molecular weight proteins or other stabilizers or excipients. The choice of a pharmaceutically acceptable carrier, including a physiologically acceptable agent, depends, for example, on the route of administration of the composition. The preparation or pharmaceutical composition can be a selfemulsifying drug delivery system or a selfmicroemulsifying drug delivery system. The pharmaceutical composition (preparation) also can be a liposome or other polymer matrix, which can have incorporated therein, for example, a compound of the invention. Liposomes, for example, which comprise phospholipids or other lipids, are nontoxic, physiologically acceptable and metabolizable carriers that are relatively simple to make and administer.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the patient. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

A pharmaceutical composition (preparation) can be administered to a subject by any of a number of routes of administration including, for example, orally (for example, drenches as in aqueous or non-aqueous solutions or suspensions, tablets, capsules (including sprinkle capsules and gelatin capsules), boluses, powders, granules, pastes for application to the tongue); absorption through the oral mucosa (e.g., sublingually); subcutaneously; transdermally (for example as a patch applied to the skin); and topically (for example, as a cream, ointment or spray applied to the skin). The compound may also be formulated for inhalation. In certain embodiments, a compound may be simply dissolved or suspended in sterile water. Details of appropriate routes of administration and compositions suitable for same can be found in, for example, U.S. Pat. Nos. 6,110,973, 5,763,493, 5,731,000, 5,541,231, 5,427,798, 5,358,970 and 4,172,896 (all incorporated by reference), as well as in patents cited therein.

The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient that can be combined with a carrier material to produce a single dosage form will generally be that amount of the compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association an active compound, such as a compound of the invention, with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules (including sprinkle capsules and gelatin capsules), cachets, pills, tablets, lozenges (using a flavored basis, usually sucrose and acacia or tragacanth), lyophile, powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. Compositions or compounds may also be administered as a bolus, electuary or paste.

To prepare solid dosage forms for oral administration (capsules (including sprinkle capsules and gelatin capsules), tablets, pills, dragees, powders, granules and the like), the active ingredient is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid, (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; (10) complexing agents, such as, modified and unmodified cyclodextrins; and (11) coloring agents. In the case of capsules (including sprinkle capsules and gelatin capsules), tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions, such as dragees, capsules (including sprinkle capsules and gelatin capsules), pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions that can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions that can be used include polymeric substances and waxes. The active ingredient can also be in micro-encapsulated form, if appropriate, with one or more of the above-described excipients.

Liquid dosage forms useful for oral administration include pharmaceutically acceptable emulsions, lyophiles for reconstitution, microemulsions, solutions, suspensions, syrups and elixirs. In addition to the active ingredient, the liquid dosage forms may contain inert diluents commonly used in the art, such as, for example, water or other solvents, cyclodextrins and derivatives thereof, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Dosage forms for the topical or transdermal administration include powders, sprays, ointments, pastes, creams, lotions, gels, solutions, patches and inhalants. The active compound may be mixed under sterile conditions with a pharmaceutically acceptable carrier, and with any preservatives, buffers, or propellants that may be required.

The ointments, pastes, creams and gels may contain, in addition to an active compound, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to an active compound, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present invention to the body. Such dosage forms can be made by dissolving or dispersing the active compound in the proper medium. Absorption enhancers can also be used to increase the flux of the compound across the skin. The rate of such flux can be controlled by either providing a rate controlling membrane or dispersing the compound in a polymer matrix or gel.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion. Pharmaceutical compositions suitable for parenteral administration comprise one or more active compounds in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers that may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents that delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which, in turn, may depend upon crystal size and crystalline form. Alternatively, delayed absorption of a parenterally administered drug form is accomplished by dissolving or suspending the drug in an oil vehicle.

Injectable depot forms are made by forming microencapsulated matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions that are compatible with body tissue.

For use in the methods of this invention, active compounds can be given per se or as a pharmaceutical composition containing, for example, 0.1 to 99.5% (more preferably, 0.5 to 90%) of active ingredient in combination with a pharmaceutically acceptable carrier.

Methods of introduction may also be provided by rechargeable or biodegradable devices. Various slow release polymeric devices have been developed and tested in vivo in recent years for the controlled delivery of drugs, including proteinaceous biopharmaceuticals. A variety of biocompatible polymers (including hydrogels), including both biodegradable and non-degradable polymers, can be used to form an implant for the sustained release of a compound at a particular target site.

Actual dosage levels of the active ingredients in the pharmaceutical compositions may be varied so as to obtain an amount of the active ingredient that is effective to achieve the desired therapeutic response for a particular patient, composition, and mode of administration, without being toxic to the patient.

The selected dosage level will depend upon a variety of factors including the activity of the particular compound or combination of compounds employed, or the ester, salt or amide thereof, the route of administration, the time of administration, the rate of excretion of the particular compound(s) being employed, the duration of the treatment, other drugs, compounds and/or materials used in combination with the particular compound(s) employed, the age, sex, weight, condition, general health and prior medical history of the patient being treated, and like factors well known in the medical arts.

A physician or veterinarian having ordinary skill in the art can readily determine and prescribe the therapeutically effective amount of the pharmaceutical composition required. For example, the physician or veterinarian could start doses of the pharmaceutical composition or compound at levels lower than that required in order to achieve the desired therapeutic effect and gradually increase the dosage until the desired effect is achieved. By “therapeutically effective amount” is meant the concentration of a compound that is sufficient to elicit the desired therapeutic effect. It is generally understood that the effective amount of the compound will vary according to the weight, sex, age, and medical history of the subject. Other factors which influence the effective amount may include, but are not limited to, the severity of the patient's condition, the disorder being treated, the stability of the compound, and, if desired, another type of therapeutic agent being administered with the compound of the invention. A larger total dose can be delivered by multiple administrations of the agent. Methods to determine efficacy and dosage are known to those skilled in the art (Isselbacher et al. (1996) Harrison's Principles of Internal Medicine 13 ed., 1814-1882, herein incorporated by reference).

In general, a suitable daily dose of an active compound used in the compositions and methods of the invention will be that amount of the compound that is the lowest dose effective to produce a therapeutic effect. Such an effective dose will generally depend upon the factors described above.

If desired, the effective daily dose of the active compound may be administered as one, two, three, four, five, six or more sub-doses administered separately at appropriate intervals throughout the day, optionally, in unit dosage forms. In certain embodiments of the present invention, the active compound may be administered two or three times daily. In preferred embodiments, the active compound will be administered once daily.

The patient receiving this treatment is any animal in need, including primates, in particular humans; and other mammals such as equines, cattle, swine, sheep, cats, and dogs; poultry; and pets in general.

In certain embodiments, compounds of the invention may be used alone or conjointly administered with another type of therapeutic agent.

The present disclosure includes the use of pharmaceutically acceptable salts of compounds of the invention in the compositions and methods of the present invention. In certain embodiments, contemplated salts of the invention include, but are not limited to, alkyl, dialkyl, trialkyl or tetra-alkyl ammonium salts. In certain embodiments, contemplated salts of the invention include, but are not limited to, L-arginine, benethamine, benzathine, betaine, calcium hydroxide, choline, deanol, diethanolamine, diethylamine, 2-(diethylamino)ethanol, ethanolamine, ethylenediamine, N-methylglucamine, hydrabamine, 1H-imidazole, lithium, L-lysine, magnesium, 4-(2-hydroxyethyl)morpholine, piperazine, potassium, 1-(2-hydroxyethyl)pyrrolidine, sodium, triethanolamine, tromethamine, and zinc salts. In certain embodiments, contemplated salts of the invention include, but are not limited to, Na, Ca, K, Mg, Zn or other metal salts. In certain embodiments, contemplated salts of the invention include, but are not limited to, 1-hydroxy-2-naphthoic acid, 2,2-dichloroacetic acid, 2-hydroxyethanesulfonic acid, 2-oxoglutaric acid, 4-acetamidobenzoic acid, 4-aminosalicylic acid, acetic acid, adipic acid, 1-ascorbic acid, 1-aspartic acid, benzenesulfonic acid, benzoic acid, (+)-camphoric acid, (+)-camphor-10-sulfonic acid, capric acid (decanoic acid), caproic acid (hexanoic acid), caprylic acid (octanoic acid), carbonic acid, cinnamic acid, citric acid, cyclamic acid, dodecylsulfuric acid, ethane-1,2-disulfonic acid, ethanesulfonic acid, formic acid, fumaric acid, galactaric acid, gentisic acid, d-glucoheptonic acid, d-gluconic acid, d-glucuronic acid, glutamic acid, glutaric acid, glycerophosphoric acid, glycolic acid, hippuric acid, hydrobromic acid, hydrochloric acid, isobutyric acid, lactic acid, lactobionic acid, lauric acid, maleic acid, 1-malic acid, malonic acid, mandelic acid, methanesulfonic acid, naphthalene-1,5-disulfonic acid, naphthalene-2-sulfonic acid, nicotinic acid, nitric acid, oleic acid, oxalic acid, palmitic acid, pamoic acid, phosphoric acid, proprionic acid, 1-pyroglutamic acid, salicylic acid, sebacic acid, stearic acid, succinic acid, sulfuric acid, 1-tartaric acid, thiocyanic acid, p-toluenesulfonic acid, trifluoroacetic acid, and undecylenic acid salts.

The pharmaceutically acceptable acid addition salts can also exist as various solvates, such as with water, methanol, ethanol, dimethylformamide, and the like. Mixtures of such solvates can also be prepared. The source of such solvate can be from the solvent of crystallization, inherent in the solvent of preparation or crystallization, or adventitious to such solvent.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: (1) water-soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal-chelating agents, such as citric acid, ethylenediamine tetraacetic acid (EDTA), sorbitol, tartaric acid, phosphoric acid, and the like.

### Definitions

Unless otherwise defined herein, scientific and technical terms used in this application shall have the meanings that are commonly understood by those of ordinary skill in the art. Generally, nomenclature used in connection with, and techniques of, chemistry, cell and tissue culture, molecular biology, cell and cancer biology, neurobiology, neurochemistry, virology, immunology, microbiology, pharmacology, genetics and protein and neutron capture agents chemistry, described herein, are those well known and commonly used in the art.

The methods and techniques of the present disclosure are generally performed, unless otherwise indicated, according to conventional methods well known in the art and as described in various general and more specific references that are cited and discussed throughout this specification. See, e.g. “Principles of Neural Science”, McGraw-Hill Medical, New York, N.Y. (2000); Motulsky, “Intuitive Biostatistics”, Oxford University Press, Inc. (1995); Lodish et al., “Molecular Cell Biology, 4th ed.”, W. H. Freeman & Co., New York (2000); Griffiths et al., “Introduction to Genetic Analysis, 7th ed.”, W. H. Freeman & Co., N.Y. (1999); and Gilbert et al., “Developmental Biology, 6th ed.”, Sinauer Associates, Inc., Sunderland, MA (2000).

Chemistry terms used herein, unless otherwise defined herein, are used according to conventional usage in the art, as exemplified by “The McGraw-Hill Dictionary of Chemical Terms”, Parker S., Ed., McGraw-Hill, San Francisco, C.A. (1985).

As used herein, the term “alkyl” refers to saturated aliphatic groups, including but not limited to C₁-C₁₀ straight-chain alkyl groups or C₁-C₁₀ branched-chain alkyl groups. Preferably, the “alkyl” group refers to C₁-C₆ straight-chain alkyl groups or C₁-C₆ branched-chain alkyl groups. Most preferably, the “alkyl” group refers to C₁-C₄ straight-chain alkyl groups or C₁-C₄ branched-chain alkyl groups. Examples of “alkyl” include, but are not limited to, methyl, ethyl, 1-propyl, 2-propyl, n-butyl, sec-butyl, tert-butyl, 1-pentyl, 2-pentyl, 3-pentyl, neo-pentyl, I-hexyl, 2-hexyl, 3-hexyl, 1-heptyl, 2-heptyl, 3-heptyl, 4-heptyl, 1-octyl, 2-octyl, 3-octyl or 4-octyl and the like. The “alkyl” group may be optionally substituted.

The term “acyl” is art-recognized and refers to a group represented by the general formula hydrocarbylC(O)—, preferably alkylC(O)—.

The term “acylamino” is art-recognized and refers to an amino group substituted with an acyl group and may be represented, for example, by the formula hydrocarbylC(O)NH—.

The term “acyloxy” is art-recognized and refers to a group represented by the general formula hydrocarbylC(O)O—, preferably alkylC(O)O—.

The term “alkoxy” refers to an alkyl group having an oxygen attached thereto. Representative alkoxy groups include methoxy, ethoxy, propoxy, tert-butoxy and the like.

The term “alkoxyalkyl” refers to an alkyl group substituted with an alkoxy group and may be represented by the general formula alkyl-O-alkyl.

The term “alkyl” refers to saturated aliphatic groups, including straight-chain alkyl groups, branched-chain alkyl groups, cycloalkyl (alicyclic) groups, alkyl-substituted cycloalkyl groups, and cycloalkyl-substituted alkyl groups. In preferred embodiments, a straight chain or branched chain alkyl has 30 or fewer carbon atoms in its backbone (e.g., C₁₋₃₀ for straight chains, C₃₋₃₀ for branched chains), and more preferably 20 or fewer.

Moreover, the term “alkyl” as used throughout the specification, examples, and claims is intended to include both unsubstituted and substituted alkyl groups, the latter of which refers to alkyl moieties having substituents replacing a hydrogen on one or more carbons of the hydrocarbon backbone, including haloalkyl groups such as trifluoromethyl and 2,2,2-trifluoroethyl, etc.

The term “C_{x-y}” or “C_{x-Cy}”, when used in conjunction with a chemical moiety, such as, acyl, acyloxy, alkyl, alkenyl, alkynyl, or alkoxy is meant to include groups that contain from x to y carbons in the chain. C₀ alkyl indicates a hydrogen where the group is in a terminal position, a bond if internal. A C₁₋₆alkyl group, for example, contains from one to six carbon atoms in the chain.

The term “alkylamino”, as used herein, refers to an amino group substituted with at least one alkyl group.

The term “alkylthio”, as used herein, refers to a thiol group substituted with an alkyl group and may be represented by the general formula alkylS—.

The term “amide”, as used herein, refers to a group

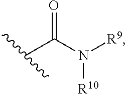

wherein R⁹ and R¹⁰ each independently represent a hydrogen or hydrocarbyl group, or R⁹ and R¹⁰ taken together with the N atom to which they are attached complete a heterocycle having from 4 to 8 atoms in the ring structure.

The terms “amine” and “amino” are art-recognized and refer to both unsubstituted and substituted amines and salts thereof, e.g., a moiety that can be represented by

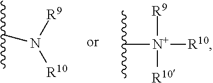

wherein R⁹, R¹⁰, and R¹⁰′ each independently represent a hydrogen or a hydrocarbyl group, or R⁹ and R¹⁰ taken together with the N atom to which they are attached complete a heterocycle having from 4 to 8 atoms in the ring structure.

The term “aminoalkyl”, as used herein, refers to an alkyl group substituted with an amino group.

The term “aralkyl”, as used herein, refers to an alkyl group substituted with an aryl group.

The term “aryl” as used herein include substituted or unsubstituted single-ring aromatic groups in which each atom of the ring is carbon. Preferably the ring is a 5- to 7-membered ring, more preferably a 6-membered ring. The term “aryl” also includes polycyclic ring systems having two or more cyclic rings in which two or more carbons are common to two adjoining rings wherein at least one of the rings is aromatic, e.g., the other cyclic rings can be cycloalkyls, cycloalkenyls, cycloalkynyls, aryls, heteroaryls, and/or heterocyclyls. Aryl groups include benzene, naphthalene, phenanthrene, phenol, aniline, and the like.

The term “carbamate” is art-recognized and refers to a group

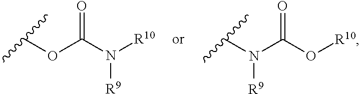

wherein R⁹ and R¹¹ independently represent hydrogen or a hydrocarbyl group.

The term “carbocyclylalkyl”, as used herein, refers to an alkyl group substituted with a carbocycle group.

The term “carbocycle” includes 5-7 membered monocyclic and 8-12 membered bicyclic rings. Each ring of a bicyclic carbocycle may be selected from saturated, unsaturated and aromatic rings. Carbocycle includes bicyclic molecules in which one, two or three or more atoms are shared between the two rings. The term “fused carbocycle” refers to a bicyclic carbocycle in which each of the rings shares two adjacent atoms with the other ring. Each ring of a fused carbocycle may be selected from saturated, unsaturated and aromatic rings. In an exemplary embodiment, an aromatic ring, e.g., phenyl, may be fused to a saturated or unsaturated ring, e.g., cyclohexane, cyclopentane, or cyclohexene. Any combination of saturated, unsaturated and aromatic bicyclic rings, as valence permits, is included in the definition of carbocyclic. Exemplary “carbocycles” include cyclopentane, cyclohexane, bicyclo[2.2.1]heptane, 1,5-cyclooctadiene, 1,2,3,4-tetrahydronaphthalene, bicyclo[4.2.0]oct-3-ene, naphthalene and adamantane. Exemplary fused carbocycles include decalin, naphthalene, 1,2,3,4-tetrahydronaphthalene, bicyclo[4.2.0]octane, 4,5,6,7-tetrahydro-1H-indene and bicyclo[4.1.0]hept-3-ene. “Carbocycles” may be substituted at any one or more positions capable of bearing a hydrogen atom.

The term “carbocyclylalkyl”, as used herein, refers to an alkyl group substituted with a carbocycle group.

The term “carbonate” is art-recognized and refers to a group —OCO₂—.

The term “carboxy”, as used herein, refers to a group represented by the formula —CO₂H.

The term “cycloalkyl” includes substituted or unsubstituted non-aromatic single ring structures, preferably 4- to 8-membered rings, more preferably 4- to 6-membered rings. The term “cycloalkyl” also includes polycyclic ring systems having two or more cyclic rings in which two or more carbons are common to two adjoining rings wherein at least one of the rings is cycloalkyl and the substituent (e.g., R¹⁰⁰) is attached to the cycloalkyl ring, e.g., the other cyclic rings can be cycloalkyls, cycloalkenyls, cycloalkynyls, aryls, heteroaryls, and/or heterocyclyls. Heteroaryl groups include, for example, pyrrole, furan, thiophene, imidazole, oxazole, thiazole, pyrazole, pyridine, pyrazine, pyridazine, pyrimidine, denzodioxane, tetrahydroquinoline, and the like.

The term “ester”, as used herein, refers to a group —C(O)OR⁹ wherein R⁹ represents a hydrocarbyl group.

The term “ether”, as used herein, refers to a hydrocarbyl group linked through an oxygen to another hydrocarbyl group. Accordingly, an ether substituent of a hydrocarbyl group may be hydrocarbyl-O—. Ethers may be either symmetrical or unsymmetrical. Examples of ethers include, but are not limited to, heterocycle-O-heterocycle and aryl-O-heterocycle. Ethers include “alkoxyalkyl” groups, which may be represented by the general formula alkyl-O-alkyl.

The terms “halo” and “halogen” as used herein means halogen and includes chloro, fluoro, bromo, and iodo.

The terms “hetaralkyl” and “heteroaralkyl”, as used herein, refers to an alkyl group substituted with a hetaryl group.

The terms “heteroaryl” and “hetaryl” include substituted or unsubstituted aromatic single ring structures, preferably 5- to 7-membered rings, more preferably 5- to 6-membered rings, whose ring structures include at least one heteroatom, preferably one to four heteroatoms, more preferably one or two heteroatoms. The terms “heteroaryl” and “hetaryl” also include polycyclic ring systems having two or more cyclic rings in which two or more carbons are common to two adjoining rings wherein at least one of the rings is heteroaromatic, e.g., the other cyclic rings can be cycloalkyls, cycloalkenyls, cycloalkynyls, aryls, heteroaryls, and/or heterocyclyls. Heteroaryl groups include, for example, pyrrole, furan, thiophene, imidazole, oxazole, thiazole, pyrazole, pyridine, pyrazine, pyridazine, and pyrimidine, and the like.

The term “heteroatom” as used herein means an atom of any element other than carbon or hydrogen. Preferred heteroatoms are nitrogen, oxygen, and sulfur.

The term “heterocyclylalkyl”, as used herein, refers to an alkyl group substituted with a heterocycle group.

The terms “heterocyclyl”, “heterocycle”, and “heterocyclic” refer to substituted or unsubstituted non-aromatic ring structures, preferably 3- to 10-membered rings, more preferably 3- to 7-membered rings, whose ring structures include at least one heteroatom, preferably one to four heteroatoms, more preferably one or two heteroatoms. The terms “heterocyclyl” and “heterocyclic” also include polycyclic ring systems having two or more cyclic rings in which two or more carbons are common to two adjoining rings wherein at least one of the rings is heterocyclic, e.g., the other cyclic rings can be cycloalkyls, cycloalkenyls, cycloalkynyls, aryls, heteroaryls, and/or heterocyclyls. Heterocyclyl groups include, for example, piperidine, piperazine, pyrrolidine, morpholine, lactones, lactams, and the like.

The term “hydrocarbyl”, as used herein, refers to a group that is bonded through a carbon atom that does not have a ═O or ═S substituent, and typically has at least one carbon-hydrogen bond and a primarily carbon backbone, but may optionally include heteroatoms. Thus, groups like methyl, ethoxyethyl, 2-pyridyl, and even trifluoromethyl are considered to be hydrocarbyl for the purposes of this application, but substituents such as acetyl (which has a ═O substituent on the linking carbon) and ethoxy (which is linked through oxygen, not carbon) are not. Hydrocarbyl groups include, but are not limited to aryl, heteroaryl, carbocycle, heterocycle, alkyl, alkenyl, alkynyl, and combinations thereof.

The term “hydroxyalkyl”, as used herein, refers to an alkyl group substituted with a hydroxy group.

The term “lower” when used in conjunction with a chemical moiety, such as, acyl, acyloxy, alkyl, alkenyl, alkynyl, or alkoxy is meant to include groups where there are ten or fewer atoms in the substituent, preferably six or fewer. A “lower alkyl”, for example, refers to an alkyl group that contains ten or fewer carbon atoms, preferably six or fewer. In certain embodiments, acyl, acyloxy, alkyl, alkenyl, alkynyl, or alkoxy substituents defined herein are respectively lower acyl, lower acyloxy, lower alkyl, lower alkenyl, lower alkynyl, or lower alkoxy, whether they appear alone or in combination with other substituents, such as in the recitations hydroxyalkyl and aralkyl (in which case, for example, the atoms within the aryl group are not counted when counting the carbon atoms in the alkyl substituent).

The terms “polycyclyl”, “polycycle”, and “polycyclic” refer to two or more rings (e.g., cycloalkyls, cycloalkenyls, cycloalkynyls, aryls, heteroaryls, and/or heterocyclyls) in which two or more atoms are common to two adjoining rings, e.g., the rings are “fused rings”. Each of the rings of the polycycle can be substituted or unsubstituted. In certain embodiments, each ring of the polycycle contains from 3 to 10 atoms in the ring, preferably from 5 to 7.

It is understood that substituents and substitution patterns on the compounds of the present invention can be selected by one of ordinary skilled person in the art to result chemically stable compounds which can be readily synthesized by techniques known in the art, as well as those methods set forth below, from readily available starting materials. If a substituent is itself substituted with more than one group, it is understood that these multiple groups may be on the same carbon or on different carbons, so long as a stable structure results.

The term “substituted” refers to moieties having substituents replacing a hydrogen on one or more carbons of the backbone. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of this invention, the heteroatoms such as nitrogen may have hydrogen substituents and/or any permissible substituents of organic compounds described herein which satisfy the valences of the heteroatoms. Substituents can include any substituents described herein, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that the moieties substituted on the hydrocarbon chain can themselves be substituted, if appropriate.

The term “modulate” as used herein includes the inhibition or suppression of a function or activity (such as cell proliferation) as well as the enhancement of a function or activity.

The phrase “pharmaceutically acceptable” is art-recognized. In certain embodiments, the term includes compositions, excipients, adjuvants, polymers and other materials and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The term “pharmaceutically acceptable salt” or “salt” is used herein to refer to an acid addition salt or a basic addition salt which is suitable for or compatible with the treatment of patients.

The term “pharmaceutically acceptable acid addition salt” as used herein means any non-toxic organic or inorganic salt of any base compounds represented by Formula I. Illustrative inorganic acids which form suitable salts include hydrochloric, hydrobromic, sulfuric and phosphoric acids, as well as metal salts such as sodium monohydrogen orthophosphate and potassium hydrogen sulfate. Illustrative organic acids that form suitable salts include mono-, di-, and tricarboxylic acids such as glycolic, lactic, pyruvic, malonic, succinic, glutaric, fumaric, malic, tartaric, citric, ascorbic, maleic, benzoic, phenylacetic, cinnamic and salicylic acids, as well as sulfonic acids such as p-toluene sulfonic and methanesulfonic acids. Either the mono or di-acid salts can be formed, and such salts may exist in either a hydrated, solvated or substantially anhydrous form. In general, the acid addition salts of compounds of Formula I are more soluble in water and various hydrophilic organic solvents, and generally demonstrate higher melting points in comparison to their free base forms. The selection of the appropriate salt will be known to one skilled in the art. Other non-pharmaceutically acceptable salts, e.g., oxalates, may be used, for example, in the isolation of compounds of Formula I for laboratory use, or for subsequent conversion to a pharmaceutically acceptable acid addition salt.

Many of the compounds useful in the methods and compositions of this disclosure have at least one stereogenic center in their structure. This stereogenic center may be present in a R or a S configuration, said R and S notation is used in correspondence with the rules described in Pure Appl. Chem. (1976), 45, 11-30. The disclosure contemplates all stereoisomeric forms such as enantiomeric and diastereoisomeric forms of the compounds, salts, prodrugs or mixtures thereof (including all possible mixtures of stereoisomers). See, e.g., WO 01/062726 (incorporated by reference).

Furthermore, certain compounds which contain alkenyl groups may exist as Z (zusammen) or E (entgegen) isomers. In each instance, the disclosure includes both mixture and separate individual isomers.

The term “agent” is used herein to denote a chemical compound (such as an organic or inorganic compound, a mixture of chemical compounds), a biological macromolecule (such as an antibody, including parts thereof as well as humanized, chimeric and human antibodies and monoclonal antibodies, a protein or portion thereof, e.g., a peptide, a lipid, a carbohydrate), or an extract made from biological materials such as bacteria, plants, fungi, or animal (particularly mammalian) cells or tissues. Agents include, for example, agents whose structure is known, and those whose structure is not known. The ability of such agents to inhibit AR or promote AR degradation may render them suitable as “therapeutic agents” in the methods and compositions of this disclosure.

A “patient,” “subject,” or “individual” are used interchangeably and refer to either a human or a non-human animal. These terms include mammals, such as humans, primates, livestock animals (including bovines, porcines, etc.), companion animals (e.g., canines, felines, etc.) and rodents (e.g., mice and rats).

“Treating” a condition or patient refers to taking steps to obtain beneficial or desired results, including clinical results. Beneficial or desired clinical results can include, but are not limited to, alleviation or amelioration of one or more symptoms or conditions, diminishment of extent of disease, stabilized (i.e. not worsening) state of disease, preventing spread of disease, delay or slowing of disease progression, amelioration or palliation of the disease state, and remission (whether partial or total), whether detectable or undetectable. “Treatment” can also mean prolonging survival as compared to expected survival if not receiving treatment.

The term “preventing” is art-recognized, and when used in relation to a condition, such as a local recurrence (e.g., pain), a disease such as cancer, a syndrome complex such as heart failure or any other medical condition, is well understood in the art, and includes administration of a composition which reduces the frequency of, or delays the onset of, symptoms of a medical condition in a subject relative to a subject which does not receive the composition. Thus, prevention of cancer includes, for example, reducing the number of detectable cancerous growths in a population of patients receiving a prophylactic treatment relative to an untreated control population, and/or delaying the appearance of detectable cancerous growths in a treated population versus an untreated control population, e.g., by a statistically and/or clinically significant amount.

“Administering” or “administration of” a substance, a compound or an agent to a subject can be carried out using one of a variety of methods known to those skilled in the art. For example, a compound or an agent can be administered, intravenously, arterially, intradermally, intramuscularly, intraperitoneally, subcutaneously, ocularly, sublingually, orally (by ingestion), intranasally (by inhalation), intraspinally, intracerebrally, and transdermally (by absorption, e.g., through a skin duct). A compound or agent can also appropriately be introduced by rechargeable or biodegradable polymeric devices or other devices, e.g., patches and pumps, or formulations, which provide for the extended, slow or controlled release of the compound or agent. Administering can also be performed, for example, once, a plurality of times, and/or over one or more extended periods.

Appropriate methods of administering a substance, a compound or an agent to a subject will also depend, for example, on the age and/or the physical condition of the subject and the chemical and biological properties of the compound or agent (e.g., solubility, digestibility, bioavailability, stability and toxicity). In some embodiments, a compound or an agent is administered orally, e.g., to a subject by ingestion. In some embodiments, the orally administered compound or agent is in an extended release or slow release formulation, or administered using a device for such slow or extended release.

As used herein, the phrase “conjoint administration” refers to any form of administration of two or more different therapeutic agents such that the second agent is administered while the previously administered therapeutic agent is still effective in the body (e.g., the two agents are simultaneously effective in the patient, which may include synergistic effects of the two agents). For example, the different therapeutic compounds can be administered either in the same formulation or in separate formulations, either concomitantly or sequentially. Tus, an individual who receives such treatment can benefit from a combined effect of different therapeutic agents.

A “therapeutically effective amount” or a “therapeutically effective dose” of a drug or agent is an amount of a drug or an agent that, when administered to a subject will have the intended therapeutic effect. The full therapeutic effect does not necessarily occur by administration of one dose, and may occur only after administration of a series of doses. Thus, a therapeutically effective amount may be administered in one or more administrations. The precise effective amount needed for a subject will depend upon, for example, the subject's size, health and age, and the nature and extent of the condition being treated, such as cancer or MDS. The skilled worker can readily determine the effective amount for a given situation by routine experimentation.

All of the above, and any other publications, patents and published patent applications referred to in this application are specifically incorporated by reference herein. In case of conflict, the present specification, including its specific definitions, will control.

### EXAMPLE

### Example 1: Synthesis

### General Information for Synthetic Experimental Section

Reagents obtained from commercial sources were used without further purification. Synthesis of the L-boroPro-pn was performed using the previously described synthetic method (T S. J. Coutts etc. J. Med. Chem. 1996, 39, 2087-2094). All the target compounds were purified by RP-HPLC using Varian semi-preparative system with a Discovery C18 569226-U RP-HPLC column. The mobile phase was typically made by mixing water (0.1% TFA) with acetonitrile (0.08% TFA) in gradient concentration. Purities determined by HPLC analysis were greater than 95%. Mass spectra and HPLC retention times were recorded on a Thermo LTQXL LC/MS system with UV detector (monitoring at 215 nm, 254 nm), using an Agilent 300SB-C8 RP-HPLC column (4.6×100 mm, 3.5 μm) with solvent gradient A) water (0.1% TFA) and B) acetonitrile (0.08% TFA) at 0.5 mL/min. Unless otherwise noted, all HPLC retention times are given for an eluent gradient 5% B for the first 3 min, then from 5% to 98% B over 6 min, which was maintained for the next 5 min. NMR spectra were recorded on a Bruker Avance 600 MHz NMR spectrometer at ambient temperature employing a 5 mm inverse multinuclear probe. Chemical shifts were reported in parts per million (δ) relative to DSS (in D₂O).

General synthetic procedure A for peptide coupling reaction with HATU. DIPEA (142 mg, 1.1 mmol) was added to a stirred mixture of N-Boc (or N-Fmoc) protected amino acid (0.5 mmol), amine (0.55 mmol), HATU (200 mg, 0.53 mmol) and anhydrous DMF (3 mL) under argon at 0° C. The reaction mixture was stirred for 15 min, warmed to room temperature, and stirred for 1 hr or until the reaction was completed. The reaction mixture was then concentrated in vacuo. The residue was re-dissolved into ethyl acetate (50 mL) or dichloromethane (50 mL) and washed sequentially with 0.1 N KHSO₄ (3×10 mL), saturated NaHCO₃ (3×10 mL), brine (10 mL), and dried over anhydrous MgSO₄, filtered and concentrated in vacuo, purified by silica gel flash chromatography to give the coupling product.

General synthetic procedure B for Boc de-protection. The Boc-protected compound (1.0 mmol) was dissolved in a 4 M solution of HCl in dioxane (5 mL), stirred for 2 hr at room temperature or until the reaction was completed, and then was concentrated in vacuo. The residue was co-evaporated with dichloromethane (3×20 mL) in vacuo to completely dry to give the unprotected amine product.

General synthetic procedure C for Fmoc de-protection. Diethylamine (3 mL) was added to a solution of The Fmoc-protected compound (1.0 mmol) in anhydrous dichloromethane (DCM, 9 mL). The reaction mixture was stirred at room temperature for 5 hr or until the reaction was completed, and then was concentrated in vacuo. The residue was co-evaporated with dichloromethane (3×20 mL) in vacuo to completely dry to give the unprotected amine product.

General synthetic procedure D for (+)-pinanediol de-protection. The (+)-pinanediol protected compound (1.0 mmol) was dissolved in cold water (10 mL). tert-Butyl methyl ether (MTBE) (20 mL) and phenylboronic acid (122 mg, 1.0 mmol) were added. The resulting mixture was stirred at room temperature for 4 hr or until the reaction was completed. The aqueous phase was separated, washed with MTBE (3×10 mL), concentrated in vacuo, and purified by semi-preparative HPLC, lyophilized to give the target product.

### Synthesis of Decaborane-Acetic Acid

The synthesis of o-Decaborane-acetic acid was conducted according to the literature reported (Jan Nekvinda, etc. Chem. Eur. J. 2018, 24, 12970-12975). Dried o-carborane (7.2 g, 50 mmol) was dissolved in dry diethyl ether (250 mL) under Ar protection. After cooling down to 0° C., n-BuLi (1.6 M in hexane, 47 mL, 1.5 eq.) was added. The reaction mixture was stirred for additional 30 minutes at room temperature and then the flask was cooled down to 0° C. again. Ethylene oxide (3.0 M in THF, 25 mL, 1.5 eq.) was then added dropwise. After stirred for 5 hours, MeOH (7.5 mL) and AcOH (0.75 mL), then water (50 mL) were added. This reaction mixture was separated, and the aqueous layer was extracted with MTBE (3×100 mL). The combined organic layers were dried over MgSO4, evaporated, and then purified by silica column chromatography (Hexane/EtOAc) to give the alcohol (5.0 g) as an oil. The alcohol (4.7 g) was dissolved in acetone (94 mL) and cooled down to 0° C. Then the CrO3 (17 g) in the mixture of AcOH (71 mL) and water (94 mL) were added dropwise. Reaction was stirred at room temperature overnight. The mixture was then extracted three times with MTBE (3×250 mL) and the combined organic layers were then acidified with 10% HCl and washed with brine (3×100 mL). The etheric phase was then dried over MgSO4, evaporated, and then purified by silica column chromatography (MeOH/DCM) to give o-Decaborane-acetic acid (2.7 g) as a pale-brown powder.

### Synthesis of Decaborane-Propionic Acid

The synthesis of o-Decaborane-propionic acid was conducted similar to the synthesis of o-Decaborane-acetic acid described above. Dried o-carborane (7.2 g, 50 mmol) was dissolved in dry diethyl ether (250 mL) under Ar protection. After cooling down to 0° C., n-BuLi (1.6 M in hexane, 47 mL, 1.5 eq.) was added. The reaction mixture was stirred for additional 30 minutes at room temperature and then the flask was cooled down to 0° C. again. Oxetane (4.9 mL) was then added dropwise. After stirred for 5 hours, MeOH (7.5 mL) and AcOH (0.75 mL), then water (50 mL) were added. This reaction mixture was separated, and the aqueous layer was extracted with MTBE (3×100 mL). The combined organic layers were dried over MgSO4, evaporated, and then purified by silica column chromatography (Hexane/EtOAc) to give the alcohol (4.0 g) as an oil. The alcohol was dissolved in acetone (80 mL) and cooled down to 0° C. Then the CrO3 (13.9 g) in the mixture of AcOH (61 mL) and water (80 mL) were added dropwise. Reaction was stirred at room temperature overnight. The mixture was then extracted three times with MTBE (3×210 mL) and the combined organic layers were then acidified with 10% HCl and washed with brine (3×100 mL). The etheric phase was then dried over MgSO4, evaporated, and then purified by silica column chromatography (MeOH/DCM) to give o-Decaborane-propionic acid (2.3 g) as a pale-yellow powder.

Synthesis of o-Carborane-1-Methylamine

The synthesis of o-caborane-1-methylamine was conducted according to the literature reported (Diego Alberti, etc. Scientific Reports. 2020, 10, 19274-12975). Decaborane (9.2 g, 75 mmol) was added to a stirred solution of propargyl phthalimide 3 (21.1 g, 114 mmol) in anhydrous toluene (163 mL) at room temperature under Ar protection. (Bmim)⁺Cl⁻ (8.0 g) was then added and the resulting mixture was heated at 90° C. for 4 hr, then cooled down, evaporated, purified by silica column chromatography (Hexane/EtOAc) to give the product (14.5 g) which was suspended in iPrOH (428 mL) and water (72 mL). NaBH₄ (8.8 g) was then added and stirred overnight under Ar at room temperature. The solvent was evaporated, and the obtained residue was re-dissolved in water (200 mL), extracted with MTBE (3×100 mL). The combined organic phases were dried with MgSO4, filtered and concentrated. The resulting solid was dissolved in 375 mL of a mixture of AcOH/HCl (4/1) and the solution was heated at 95° C. for 2 hr. The solvents were evaporated, and the residue was suspended in DCM and stirred for 3 hr at rt. Finally, the suspension was filtered, and the recovered white solid was washed with more DCM (3×50 mL) and dried to give o-caborane-1-methylamine hydrochloride salt (7.0 g).

### Synthesis of Compound 7349

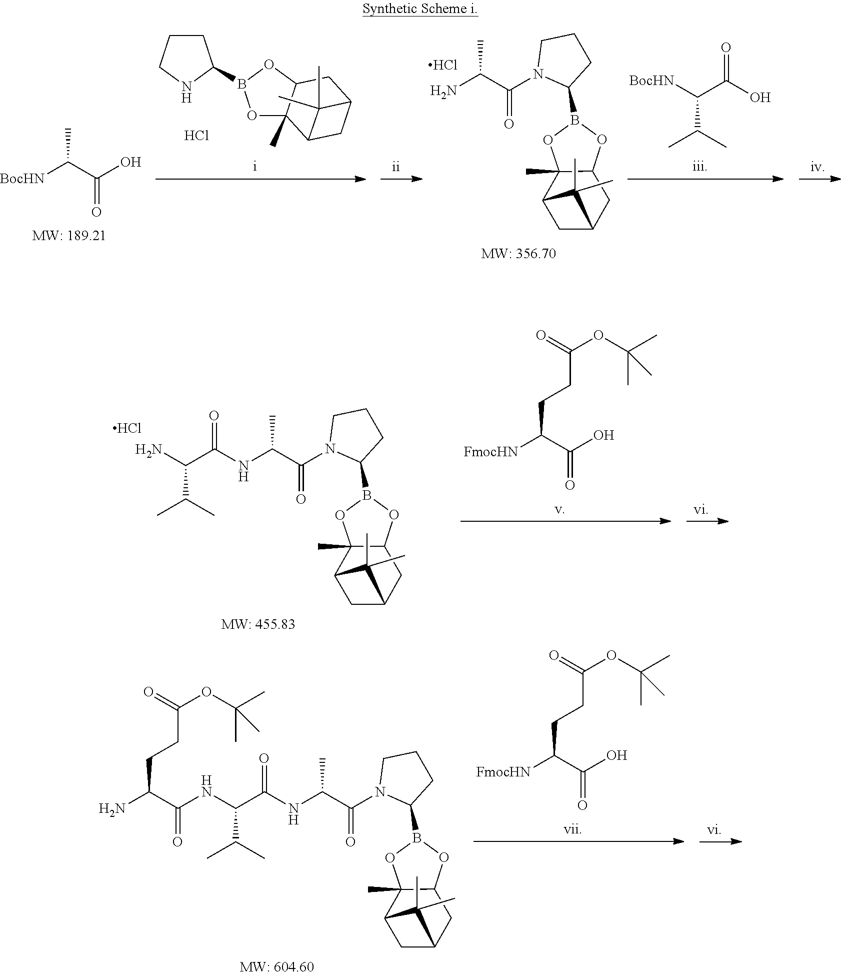

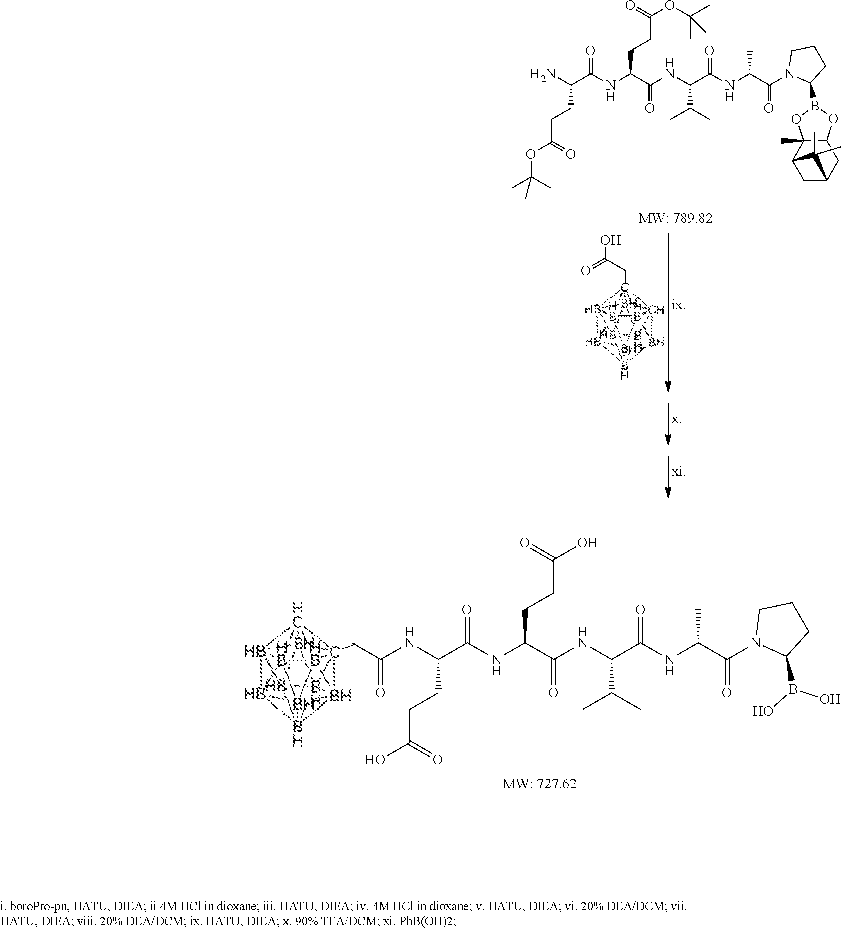

### Synthesis of H-D-Ala-boroPro-Pn·HCl

Followed the General procedure A and B: To a stirred solution of N-Boc-D-Ala-OH (1.9 g, 10 mmol) in anhydrous DMF (40 mL) was added L-boroPro-pn·HCl (3.0 g, 10.5 mmol), HATU (4.0 g, 10.5 mmol) and DIEA (4.0 mL, 23 mmol) under ice-water bath cooling. The resulting mixture was stirred at room temperature for 2 hr and then condensed in vacuo. The residue was dissolved with ethyl acetate (150 mL), washed sequentially by 0.1N KHSO₄ (3×40 mL), aq. NaHCO₃ (3×40 mL), brine (30 mL). The organic phase was dried over anhydrous MgSO₄, filtered, and evaporated in vacuo to give N-Boc-D-Ala-L-boroPro-pn which was then added to a solution of 4 N HCl in dioxane (30 mL) under ice-water cooling. The resulting mixture was stirred at room temperature for 2 hr and then condensed in vacuo. The residue was co-evaporated with dichloromethane (3×30 mL) in vacuo to completely dry. Compound H-D-Ala-boroPro-Pn·HCl was thus obtained as a white powder (3.3 g).

### Synthesis of H-Val-D-Ala-boroPro-Pn·HCl

Followed the General procedure A and B: To a stirred solution of N-Boc-Val-OH (1.24 g, 5.7 mmol) in anhydrous DMF (6 mL) and anhydrous DCM (23 mL) was added HATU (2.28 g, 6 mmol), D-Ala-boroPro-pn·HCl (2.14 g, 6 mmol), and DIEA (2.2 mL) under ice-water bath cooling. The resulting mixture was stirred at room temperature for 2 hr and then condensed in vacuo. The residue was dissolved with DCM (180 mL), washed sequentially by 0.5N KHSO₄ (3×60 mL), aq. NaHCO₃ (3×60 mL), brine (60 mL). The organic phase was dried over anhydrous MgSO4, filtered, and evaporated in vacuo to give N-Boc-Val-D-Ala-L-boroPro-pn which was then added to a solution of 4N HCl in dioxane (30 mL) under ice-water cooling. The resulting mixture was stirred at room temperature for 2 hr and then condensed in vacuo. The residue was co-evaporated with dichloromethane (3×30 mL) in vacuo to completely dry. Compound H-V71-D-Ala-boroPro-Pn·HCl was thus obtained as a white powder (2.8 g).

### Synthesis of H-Glu(OtBu)-Glu(OtBu)-Val-D-Ala-boroNo-Pn

To a stirred solution of N-Fmoc-Glu(OtBu)-OH (0.61 g, 1.43 mmol) in anhydrous DMF (7 mL) was added HATU (0.57 g, 1.5 mmol), Val-D-Ala-boroPro-pn·HCl (0.68 g, 1.5 mmol), and DIEA (0.55 mL) under ice-water bath cooling. The resulting mixture was stirred at room temperature for 2 hr and then condensed in vacuo. The residue was dissolved with DCM (60 mL), washed sequentially by 0.5N KHSO₄ (3×15 mL), aq. NaHCO₃ (3×15 mL), brine (15 mL). The organic phase was dried over anhydrous MgSO₄, filtered, and evaporated in vacuo to give N-Fmoc-Glu(OtBu)-Val-D-Ala-L-boroPro-pn which was then treated with 20% of DEA in DCM (7 mL). The resulting mixture was stirred at room temperature overnight, and then condensed in vacuo. The residue was co-evaporated with dichloromethane (3×15 mL) in vacuo to completely dry, then triturated with Hexane (50 mL), decanted the upper layer, evaporated again to give H-Glu(OtBu)-Val-D-Ala-boroPro-pn (0.9 g).

To a stirred solution of N-Fmoc-Glu(OtBu)-OH (0.58 g, 1.36 mmol) in anhydrous DMF (7 mL) was added HATU (0.54 g, 1.43 mmol), H-Glu(OtBu)-Val-D-Ala-boroPro-pn (0.86 g, 1.43 mmol), and DIEA (0.29 mL) under ice-water bath cooling. The resulting mixture was stirred at room temperature for 2 hr and then condensed in vacuo. The residue was dissolved with DCM (60 mL), washed sequentially by 0.5N KHSO₄ (3×15 mL), aq. NaHCO₃ (3×15 mL), brine (15 mL). The organic phase was dried over anhydrous MgSO₄, filtered, and evaporated in vacuo to give N-Fmoc-Glu(OtBu)-Glu(OtBu)-Val-D-Ala-L-boroPro-pn which was then treated with 20% of DEA in DCM (7 mL). The resulting mixture was stirred at room temperature overnight, and then condensed in vacuo. The residue was co-evaporated with dichloromethane (3×15 mL) in vacuo to completely dry, then triturated with Hexane (50 mL), decanted the upper layer, evaporated again to give H-Glu(OtBu)-Glu(OtBu)-Val-D-Ala-boroPro-pn (1.1 g).

### Synthesis of Decaborane-Acetyl-Glu-Glu-Val-D-Ala-boroPro (Compound 7349)

To a stirred solution of o-decaborane-acetic acid (0.263 g, 1.3 mmol) in anhydrous DMF (6.5 mL) was added HATU (0.517 g, 1.36 mmol), H-Glu(OtBu)-Glu(OtBu)-Val-D-Ala-boroPro-pn (0.9 g, 1.36 mmol), and DIEA (0.28 mL) under ice-water bath cooling. The resulting mixture was stirred at room temperature for 2 hr and then condensed in vacuo. The residue was dissolved with DCM (60 mL), washed sequentially by 0.5N KHSO₄ (3×15 mL), aq. NaHCO₃ (3×15 mL), brine (15 mL). The organic phase was dried over anhydrous MgSO₄, filtered, and evaporated in vacuo to give Decaborane-acetyl-Glu(OtBu)-Glu(OtBu)-Val-D-Ala-L-boroPro-pn which was then treated with 90% TFA in DCM (10 mL) under ice-water cooling. The resulting mixture was stirred at room temperature for 2.5 hr and then condensed in vacuo. The residue was co-evaporated with dichloromethane (3×20 mL) in vacuo to completely dry. Cold water (50 mL) and acetonitrile (20 mL) were added. The pH was adjusted to 1.5 with 5% HCL Hexanes (100 mL) and phenylboronic acid (317 mg) were added. The resulting mixture was stirred at room temperature for 3 hr. The aqueous phase was separated, washed with hexanes (2×15 mL), concentrated in vacuo, and purified by semi-preparative HPLC, lyophilized to give the target product. LC-MS (ESI⁺) m/z (rel intensity): 1402.02 (100), 710.31 ([M−H₂O+H]⁺, 84), 693.56 (40); tr=8.97 min.

### Synthesis of Compound 7388

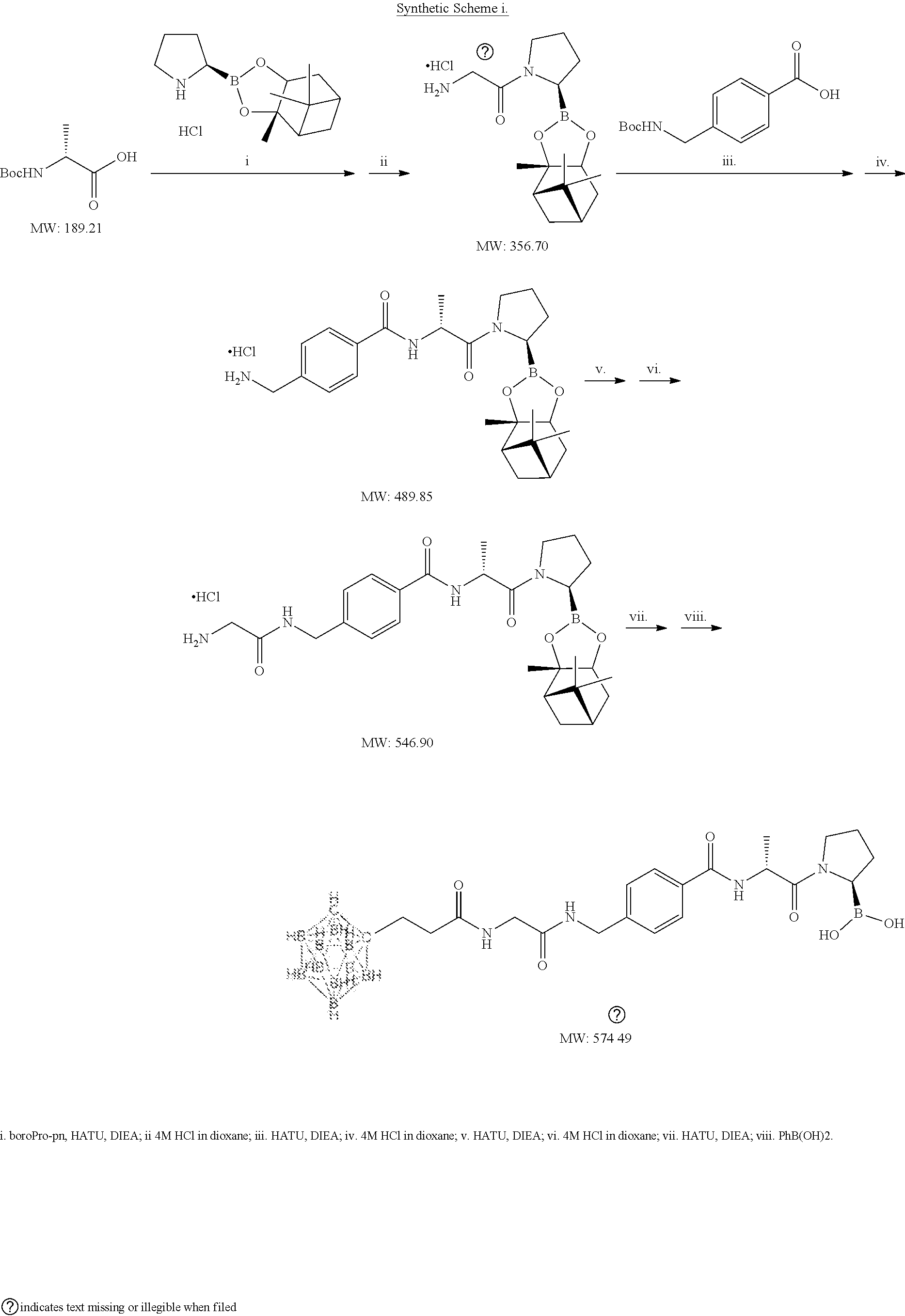

### Synthesis of AMB-D-Ala-boroPro-pn·HCl

To a stirred solution of 4-[(tert-butoxycarbonylamino)methyl]benzoic acid (505 mg, 2 mmol) in anhydrous DMF (20 mL) was added HATU (800 mg, 2.1 mmol), DIEA (0.80 mL, 4.6 mmol) and H-D-Ala-boroPro-pn·HCl (750 mg, 2.1 mmol) under ice-water bath cooling. The resulting mixture was stirred at room temperature for 2 hr and then condensed in vacuo. The residue was dissolved with dichloromethane (100 mL), washed sequentially by 0.1N KHSO₄ (3×15 mL), aq. NaHCO₃ (3×15 mL), brine (10 mL). The organic phase was dried over anhydrous MgSO4, filtered, and evaporated in vacuo to give 4-(N-Boc-aminomethyl)-PhCO-D-Ala-L-boroPro-pn which was purified by silica gel flash chromatography eluted with Ethyl Acetate/Hexanes; and then added to a solution of 4N HCl in dioxane (10 mL) under ice-water cooling. The resulting mixture was stirred at room temperature for 2 hr and then condensed in vacuo. The residue was co-evaporated with dichloromethane (3×20 mL) in vacuo to completely dry. AMB-D-Ala-boroPro-pn·HCl was thus obtained as a white powder (830 mg). LC-MS (ESI⁺) m/z (rel intensity): 453.7 ([M+H]⁺, 100); tr=9.0 min.

### Synthesis of H-Gly-AMB-D-Ala-boroPro-pn·HCl

To a stirred solution of N-Boc-Gly-OH (270 mg, 1.54 mmol) in anhydrous DMF (20 mL) was added HATU (620 mg), DIEA (0.80 mL) and H-AMB-D-Ala-boroPro-pn·HCl (980 mg) under ice-water bath cooling. The resulting mixture was stirred at room temperature for 2 hr and then condensed in vacuo. The residue was dissolved with ethyl acetate (80 mL), washed sequentially by 0.25 N KHSO₄ (3×20 mL), aq. NaHCO₃ (3×20 mL), brine (20 mL). The organic phase was dried over anhydrous MgSO₄, filtered, and evaporated in vacuo, and was then added to a solution of 4N HC in dioxane (20 mL) under ice-water cooling. The resulting mixture was stirred at room temperature for 2 hr and then condensed in vacuo. The residue was co-evaporated with dichloromethane (3×30 mL) in vacuo to completely dry. H-Gly-AMB-D-Ala-boroPro-pn·HCl was thus obtained as a yellow solid (0.8 g).

### Synthesis of Decaborane-Propionyl-Gly-AMB-D-Ala-boroPro (Compound 7388)

To a stirred solution of o-decaborane-propionic acid (151 mg, 0.7 mmol) in anhydrous DMF (10 mL) was added HATU (278 mg), DIEA (0.27 mL) and H-Gly-AMB-D-Ala-boroPro-pn·HCl (400 mg) under ice-water bath cooling. The resulting mixture was stirred at room temperature for 2 hr and then condensed in vacuo. The residue was dissolved with ethyl acetate (40 mL), washed sequentially by 0.25 N KHSO₄ (3×15 mL), aq. NaHCO₃ (3×15 mL), brine (2×15 mL). The organic phase was dried over anhydrous MgSO₄, filtered, and evaporated in vacuo to give Decaborane-propionyl-Gly-AMB-D-Ala-L-boroPro-pn (0.48 g). Water (40 mL) and acetonitrile (20 mL) were added. The pH was adjusted to 1.5 with 5% HCl. Hexanes (80 mL) and phenylboronic acid (171 mg) were added. The resulting mixture was stirred at room temperature for 3 hr. The aqueous phase was separated, washed with hexanes (2×10 mL), concentrated in vacuo, and purified by semi-preparative HPLC, lyophilized to give the target product. LC-MS (ESI⁺, Instrumentation: Agilent 1290 HPLC/6460 Triple Quad LC/MS; column: Zorbax Eclipse Plus C18, 2.1×50 mm, 1.8 μm, HPLC method: Mobile Phase A: 0.1% TFA in water, Mobile Phase B: 0.08% TFA in ACN; Flow rate: 0.5 mL/min, Gradient: 0-1 min, 10% B; 5-6 min, 98% B) m/z (rel intensity): 557.6 ([M−H₂O+H]⁺, 100); tr=3.626 min.

### Synthesis of Compound 7034

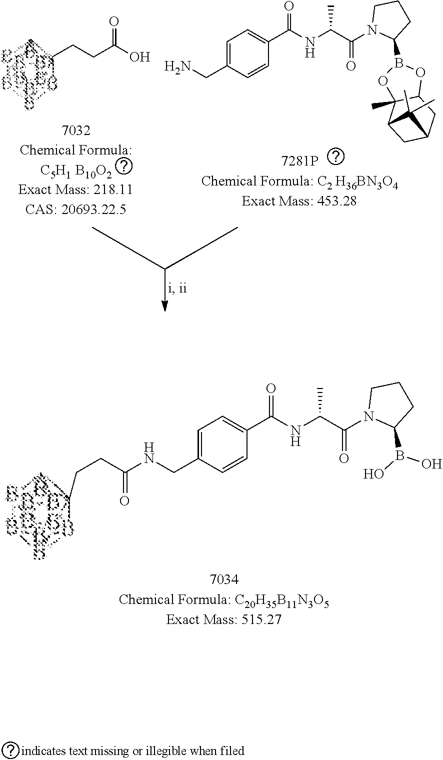

Synthetic Scheme: i. HATU, DIEA, DMF: ii. PhB(OH)2.

### Synthesis of Decaborane-Propionyl-AMB-D-Ala-boroPro (Compound 7034)

To a stirred solution of o-decaborane-propionic acid (54 mg, 0.25 mmol) in anhydrous DMF (3 mL) was added HATU (100 mg), DIEA (0.2 mL) and H-AMB-D-Ala-boroPro-pn·HCl (125 mg) under ice-water bath cooling. The resulting mixture was stirred at room temperature for 2 hr and then condensed in vacuo. The residue was dissolved with ethyl acetate (30 mL), washed sequentially by 0.1 N KHSO₄ (3×10 mL), aq. NaHCO₃ (3×10 mL), brine (10 mL). The organic phase was dried over anhydrous MgSO₄, filtered, and evaporated in vacuo to give Decaborane-propionyl-AMB-D-Ala-L-boroPro-pn. Water (10 mL) and acetonitrile (5 mL) were added. The pH was adjusted to 2 with 1N TFA. MTBE (25 mL) and phenylboronic acid (33 mg) were added. The resulting mixture was stirred at room temperature overnight. The aqueous phase was separated, washed with MTBE (2×10 mL), concentrated in vacuo, and purified by semi-preparative HPLC, lyophilized to give the target product. LC-MS (ESI⁺, with ZORBAX Eclipse Plus C18 RP-HPLC column (4.6×50 mm, 1.8 μm), eluent gradient 10% B for the first 3 min, then from 10% to 98% B over 7 min, which was maintained for the next 5 min) m/z (rel intensity): 1000.06 (75), 500.63 ([M−H₂O+H]⁺, 100); tr=8.92 min.

### Synthesis of Compound 7350

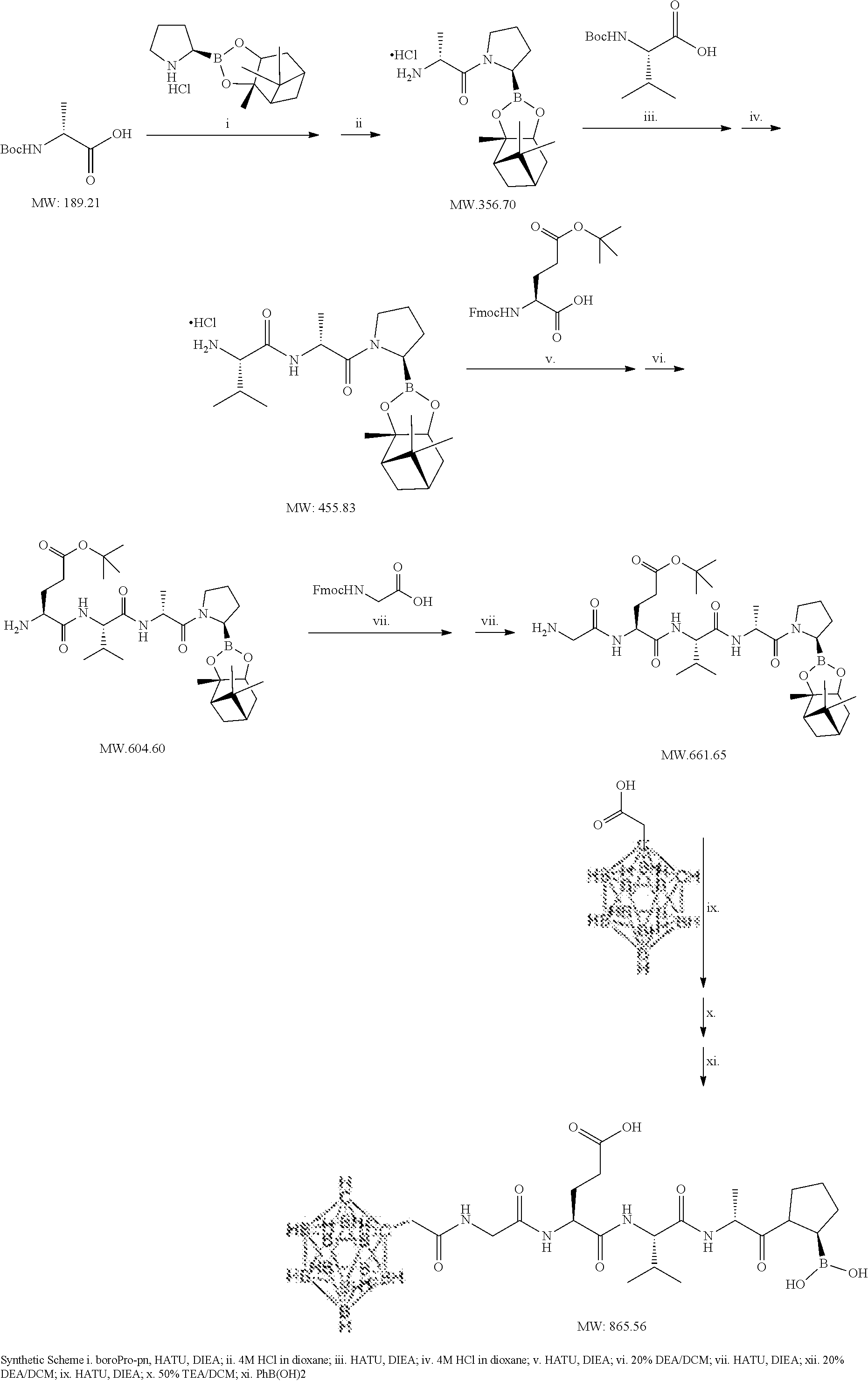

### Synthesis of H-Gly-Glu(OtBu)-Val-D)-Ala-boroPro-Pn

To a stirred solution of N-Fmoc-Gly-OH (0.4 g, 1.36 mmol) in anhydrous DMF (7 mL) was added HATU (0.54 g, 1.43 mmol), H-Glu(OtBu)-Val-D-Ala-boroPro-pn (0.86 g, 1.43 mmol), and DIEA (0.29 mL) under ice-water bath cooling. The resulting mixture was stirred at room temperature for 2 hr and then condensed in vacuo. The residue was dissolved with DCM (60 mL), washed sequentially by 0.5N KHSO₄ (3×15 mL), aq. NaHCO₃ (3×15 mL), brine (15 mL). The organic phase was dried over anhydrous MgSO₄, filtered, and evaporated in vacuo to give N-Fmoc-Gly-Glu(OtBu)-Val-D-Ala-L-boroPro-pn which was then treated with 20% of DEA in DCM (7 mL). The resulting mixture was stirred at room temperature overnight, and then condensed in vacuo. The residue was co-evaporated with dichloromethane (3×10 mL) in vacuo to completely dry, then triturated with Hexane (50 mL), filtered, and dried to give H-Gly-Glu(OtBu)-Val-D-Ala-boroPro-pn (0.9 g).

### Synthesis of Decaborane-Acetyl-Gly-Glu-Val-D-Ala-boroPro (Compound 7350)

To a stirred solution of o-decaborane-acetic acid (0.263 g, 1.3 mmol) in anhydrous DMF (6.5 mL) was added HATU (0.517 g, 1.36 mmol), H-Gly-Glu(OtBu)-Val-D-Ala-boroPro-pn (0.9 g), and DIEA (0.28 mL) under ice-water bath cooling. The resulting mixture was stirred at room temperature for 2 hr and then condensed in vacuo. The residue was dissolved with DCM (60 mL), washed sequentially by 0.5N KHSO₄ (3×15 mL), aq. NaHCO₃ (3×15 mL), brine (15 mL). The organic phase was dried over anhydrous MgSO₄, filtered, and evaporated in vacuo to give Decaborane-acetyl-Gly-Glu(OtBu)-Val-D-Ala-L-boroPro-pn which was then treated with 90% TFA in DCM (10 mL) under ice-water cooling. The resulting mixture was stirred at room temperature for 2.5 hr and then condensed in vacuo. The residue was co-evaporated with dichloromethane (3×20 mL) in vacuo to completely dry. Cold water (50 mL) and acetonitrile (20 mL) were added. The pH was adjusted to 1.5 with 5% HCL. Hexanes (100 mL) and phenylboronic acid (317 mg, 1.0 mmol) were added. The resulting mixture was stirred at room temperature for 3 hr. The aqueous phase was separated, washed with hexanes (2×15 mL), concentrated in vacuo, and purified by semi-preparative HPLC, lyophilized to give the target product. LC-MS (ESI⁺) m-z (rel intensity): 1258.03 (30), 638.37 ([M−H₂O+H]⁺, 100), 620.55 (72); tr=9.10 min.

### Synthesis of Compound 7412

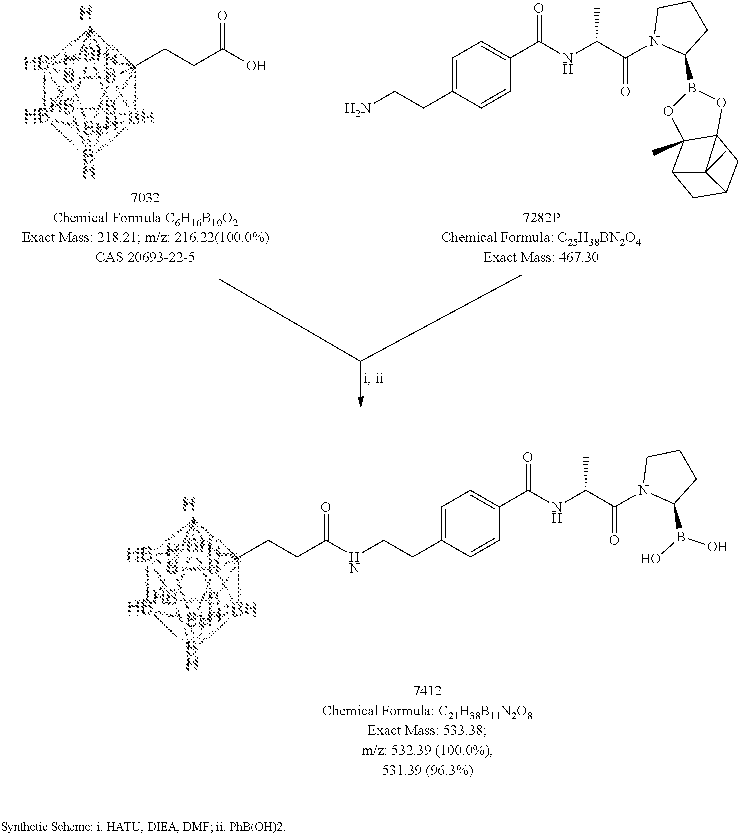

### Synthesis of AminoEthylbenzoyl-D-Ala-boroPro-pn·HCl

To a stirred solution of 4-[(tert-butoxycarbonylamino)ethyl]benzoic acid (533 mg, 2 mmol) in anhydrous DMF (20 mL) was added HATU (800 mg, 2.1 mmol), DIEA (0.80 mL, 4.6 mmol) and H-D-Ala-boroPro-pn·HCl (750 mg, 2.1 mmol) under ice-water bath cooling. The resulting mixture was stirred at room temperature for 2 hr and then condensed in vacuo. The residue was dissolved with dichloromethane (100 mL), washed sequentially by 0.1N KHSO₄ (3×15 mL), aq. NaHCO₃ (3×15 mL), brine (10 mL). The organic phase was dried over anhydrous MgSO₄, filtered, and evaporated in vacuo to give 4-(N-Boc-aminoethyl)-PhCO-D-Ala-L-boroPro-pn which was purified by silica gel flash chromatography eluted with Ethyl Acetate/Hexanes; and then added to a solution of 4N HCl in dioxane (10 mL) under ice-water cooling. The resulting mixture was stirred at room temperature for 2 hr and then condensed in vacuo. The residue was co-evaporated with dichloromethane (3×20 mL) in vacuo to completely dry to give H-AminoEthylbenzoyl-D-Ala-boroPro-pn·HCl as a white powder (800 mg).

### Synthesis of Decaborane-Propionyl-Aminoethylbenzoyl-D-Ala-boroPro (Compound 7412)

To a stirred solution of o-decaborane-propionic acid (54 mg, 0.25 mmol) in anhydrous DMF (3 mL) was added HATU (100 mg), DIEA (0.2 mL) and H-AminoEthylbenzoyl-D-Ala-boroPro-pn·HCl (130 mg) under ice-water bath cooling. The resulting mixture was stirred at room temperature for 2 hr and then condensed in vacuo. The residue was dissolved with ethyl acetate (30 mL), washed sequentially by 0.1 N KHSO₄ (3×10 mL), aq. NaHCO₃ (3×10 mL), brine (10 mL). The organic phase was dried over anhydrous MgSO4, filtered, and evaporated in vacuo to give Decaborane-propionyl-Aminoethylbenzoyl-D-Ala-L-boroPro-pn. Water (10 mL) and acetonitrile (5 mL) were added. The pH was adjusted to 2 with 1N TFA. MTBE (25 mL) and phenylboronic acid (33 mg) were added. The resulting mixture was stirred at room temperature overnight. The aqueous phase was separated, washed with MTBE (2×10 mL), concentrated in vacuo, and purified by semi-preparative HPLC, lyophilized to give the target product. LC-MS (ESI⁺, with ZORBAX Eclipse Plus C18 RP-HPLC column (4.6×50 mm, 1.8 μm), eluent gradient 10% B for the first 3 min, then from 10% to 98% B over 7 min, which was maintained for the next 5 min) m/z (rel intensity): 1028.03 (72), 514.77 ([M−H₂O+H]⁺, 100); tr=9.00 min.

### Synthesis of Compound 7282

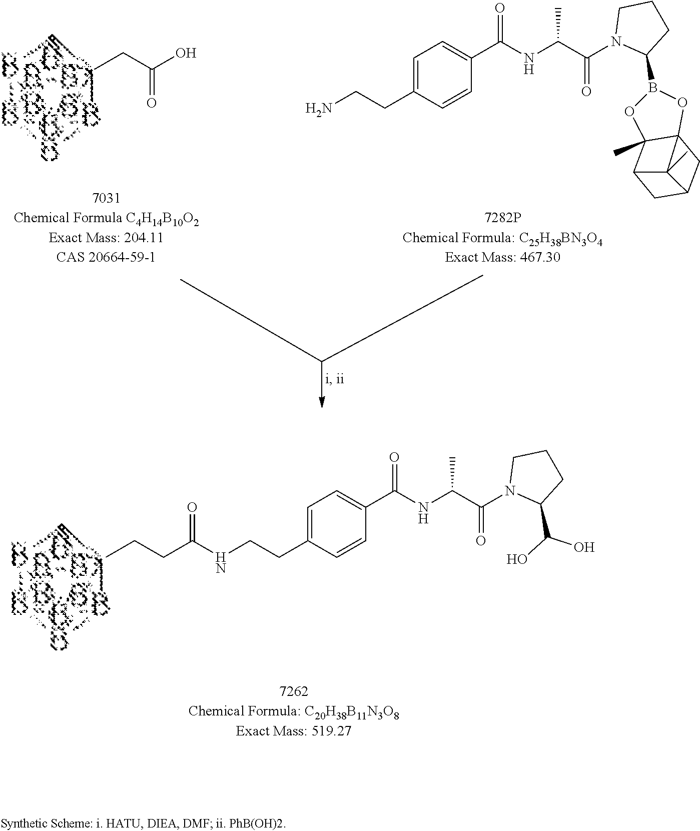

### Synthesis of Decaborane-Acetyl-Aminoethylbenzoyl-D-Ala-boroPro (Compound 7282)

To a stirred solution of o-decaborane-acetic acid (51 mg, 0.25 mmol) in anhydrous DMF (3 mL) was added HATU (100 mg), DIEA (0.2 mL) and H-AminoEthylbenzoyl-D-Ala-boroPro-pn·HCl (130 mg) under ice-water bath cooling. The resulting mixture was stirred at room temperature for 2 hr and then condensed in vacuo. The residue was dissolved with ethyl acetate (30 mL), washed sequentially by 0.1 N KHSO₄ (3×10 mL), aq. NaHCO₃ (3×10 mL), brine (10 mL). The organic phase was dried over anhydrous MgSO₄, filtered, and evaporated in vacuo to give Decaborane-acetyl-Aminoethylbenzoyl-D-Ala-L-boroPro-pn. Water (10 mL) and acetonitrile (5 mL) were added. The pH was adjusted to 2 with 1N TFA. MTBE (25 mL) and phenylboronic acid (33 mg) were added. The resulting mixture was stirred at room temperature overnight. The aqueous phase was separated, washed with MTBE (2×10 mL), concentrated in vacuo, and purified by semi-preparative HPLC, lyophilized to give the target product. LC-MS (ESI⁺, with ZORBAX Eclipse Plus C18 RP-HPLC column (4.6×50 mm, 1.8 μm), eluent gradient 10% B for the first 3 min, then from 10% to 98% B over 7 min, which was maintained for the next 5 min) m/z (rel intensity): 999.96 (37), 500.61 ([M−H₂O+H]⁺, 100); tr=8.97 min. ¹H NMR (D₂O/CAN-d3): δ 1.49 (d, J=7.2 Hz, 3H), 1.75-2.50 (m, 22H), 2.95-3.00 (m, 2H), 3.07-3.10 (m, 1H), 3.14 (s, 2H), 3.58-3.60 (m, 2H), 3.65-3.72 (m, 2H), 4.44 (s, 1H), 7.49 (d, J=8.4 Hz, 2H), 7.89 (d, J=7.8 Hz, 2H), 8.15 (s, 1H).

### Synthesis of Compound 7430B

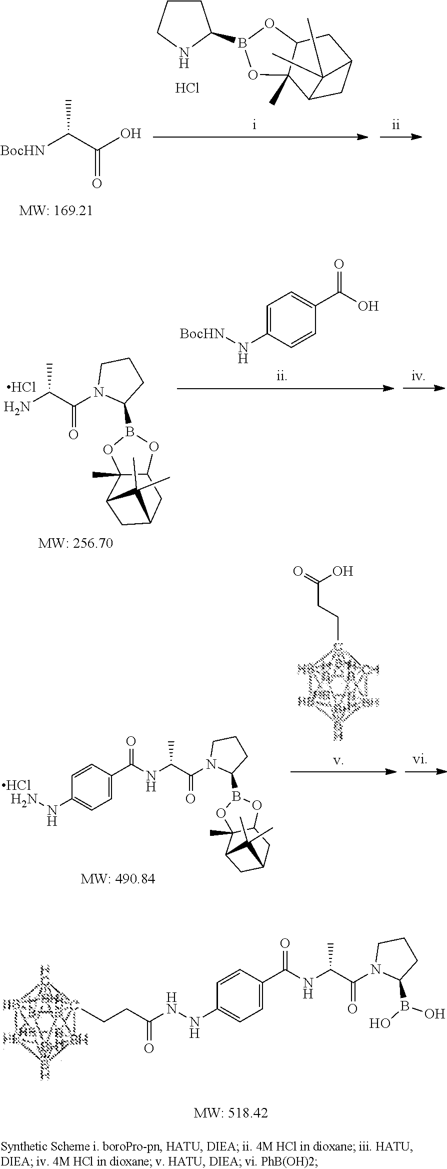

### Synthesis of Hydrazinyl-Benzoyl-D-Ala-boroPro-Pn·HCl

To a stirred solution of 4-[N-Boc-hydrazinyl l]benzoic acid (252 mg, 1 mmol) in anhydrous DMF (10 mL) was added HATU (400 mg), DIEA (0.40 mL) and H-D-Ala-boroPro-pn·HCl (395 mg) under ice-water bath cooling. The resulting mixture was stirred at room temperature for 2 hr and then condensed in vacuo. The residue was dissolved with ethyl acetate (50 mL), washed sequentially by 0.25N KHSO₄ (3×10 mL), aq. NaHCO₃ (3×10 mL), brine (10 mL). The organic phase was dried over anhydrous MgSO₄, filtered, and evaporated in vacuo to give 4-(N-Boc-hydrazinyl)-PhCO-D-Ala-L-boroPro-pn which was then added to a solution of 4N HCl in dioxane (15 mL) under ice-water cooling. The resulting mixture was stirred at room temperature for 2 hr and then condensed in vacuo. The residue was co-evaporated with dichloromethane (3×20 mL) in vacuo to completely dry to give H-4-(hydrazinyl)-benzoyl-D-Ala-boroPro-pn·HCl as a yellow powder (480 mg).

### Synthesis of 4-(Decaborane-Propionyl-Hydrazinyl)-Benzoyl-D-Ala-boroPro (Compound 7430B)

To a stirred solution of o-decaborane-propionic acid (167 mg, 0.77 mmol) in anhydrous DMF (10 mL) was added HATU (307 mg), DIEA (0.3 mL) and H-4-(hydrazinyl)-benzoyl-D-Ala-boroPro-pn·HCl (400 mg) under ice-water bath cooling. The resulting mixture was stirred at room temperature for 2 hr and then condensed in vacuo. The residue was dissolved with ethyl acetate (40 mL), washed sequentially by 0.25 N KHSO₄ (3×10 mL), aq. NaHCO₃ (3×10 mL), brine (3×10 mL). The organic phase was dried over anhydrous MgSO₄, filtered, and evaporated in vacuo to give 4-(Decaborane-propionyl-hydrazinyl)-benzoyl-D-Ala-L-boroPro-pn. Water (40 mL) and acetonitrile (20 mL) were added. The pH was adjusted to 1.5 with 5% HCL Hexanes (80 mL) and phenylboronic acid (188 mg) were added. The resulting mixture was stirred for 3 hr at room temperature. The aqueous phase was separated, washed with hexane (2×10 mL), concentrated in vacuo, and purified by semi-preparative HPLC, lyophilized to give the target product. LC-MS (ESI⁺, Instrumentation: Agilent 1290 HPLC/6460 Triple Quad LC/MS; column: Zorbax Eclipse Plus C18, 2.1×50 mm, 1.8 μm; HPLC method: Mobile Phase A: 0.1% TFA in water, Mobile Phase B: 0.08% TFA in ACN; Flow rate: 0.4 mL/min, Gradient: 0-1 min, 2% B; 5-6 min, 98% B) m/z (rel intensity): 501.4 ([M−H₂O+H]⁺, 100); tr=3.940 min.

### Synthesis of Compound 7035

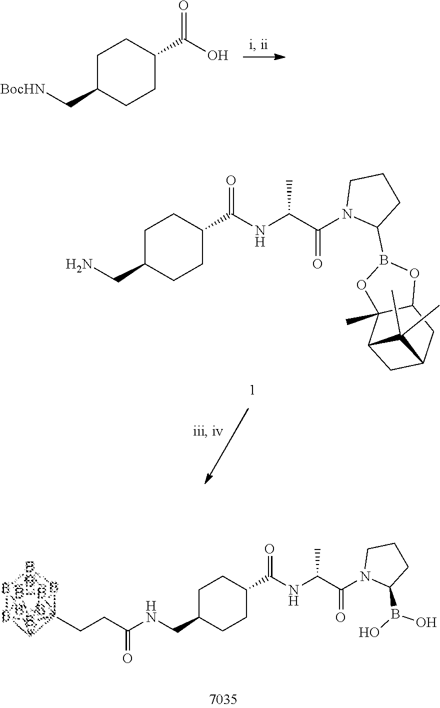

### Synthesis of H-TXA-D-Ala-boroPro-pn·HCl

To a stirred solution of trans-4-(tert-Butoxycarbonylaminomethyl)cyclohexanecarboxylic acid (TCI, B3253; 515 mg, 2 mmol) in anhydrous DMF (8 mL) was added Compound 1 (750 mg, 2.1 mmol), HATU (800 mg, 2.1 mmol) and DIEA (0.80 mL, 4.6 mmol) under ice-water bath cooling. The resulting mixture was stirred at room temperature for 2 hr and then condensed in vacuo. The residue was dissolved with dichloromethane (100 mL), washed sequentially by 0.1N KHSO₄ (3×15 mL), aq. NaHCO₃ (3×15 mL), brine (10 mL). The organic phase was dried over anhydrous MgSO₄, filtered, and evaporated in vacuo to give the N-Boc-TXA-D-Ala-boroPro-pn which was purified by silica gel flash chromatography eluted with Ethyl Acetate/Hexanes; and then added to a solution of 4N HCl in dioxane (10 mL) under ice-water cooling. The resulting mixture was stirred at room temperature for 2 hr and then condensed in vacuo. The residue was co-evaporated with dichloromethane (3×20 mL) in vacuo to completely dry. H-TXA-D-Ala-boroPro-pn·HCl was thus obtained as a white powder (890 mg). LC-MS (ESI⁺) m/z (rel intensity): 459.9 ([M+H]⁺, 100); tr=8.9 min.

### Synthesis of Decaborane-Propionyl-TXA-D-Ala-boroPro (Compound 7035)

To a stirred solution of o-decaborane-propionic acid (205 mg, 0.95 mmol) in anhydrous DMF (10 mL) was added HATU (380 mg), DIEA (0.36 mL) and H-TXA-D-Ala-boroPro-pn·HCl (496 mg) under ice-water bath cooling. The resulting mixture was stirred at room temperature for 2 hr and then condensed in vacuo. The residue was dissolved with ethyl acetate (50 mL), washed sequentially by 0.25 N KHSO₄ (3×10 mL), aq. NaHCO₃ (3×10 mL), brine (3×10 mL). The organic phase was dried over anhydrous MgSO₄, filtered, and evaporated in vacuo to give Decaborane-propionyl-TXA-D-Ala-L-boroPro-pn. Water (40 mL) and acetonitrile (20 mL) were added. The pH was adjusted to 1.5 with 5% HCl. Hexanes (80 mL) and phenylboronic acid (244 mg) were added. The resulting mixture was stirred for 3 hr at room temperature. The aqueous phase was separated, washed with hexane (2×10 mL), concentrated in vacuo, and purified by semi-preparative HPLC, lyophilized to give the target product. LC-MS (ESI⁺, Instrumentation: Agilent 1290 HPLC/6460 Triple Quad LC/MS; column: Zorbax Eclipse Plus C18, 2.1×50 mm, 1.8 μm; HPLC method: Mobile Phase A: 0.1% TFA in water, Mobile Phase B: 0.08% TFA in ACN; Flow rate: 0.4 mL/min, Gradient: 0-1 min, 2% B; 5-6 min, 98% B) m4 (rel intensity): 1012.3 (57), 506.6 ([M−H₂O+H]⁺, 100); tr=3.894 min.

### Synthesis of Compound 7348

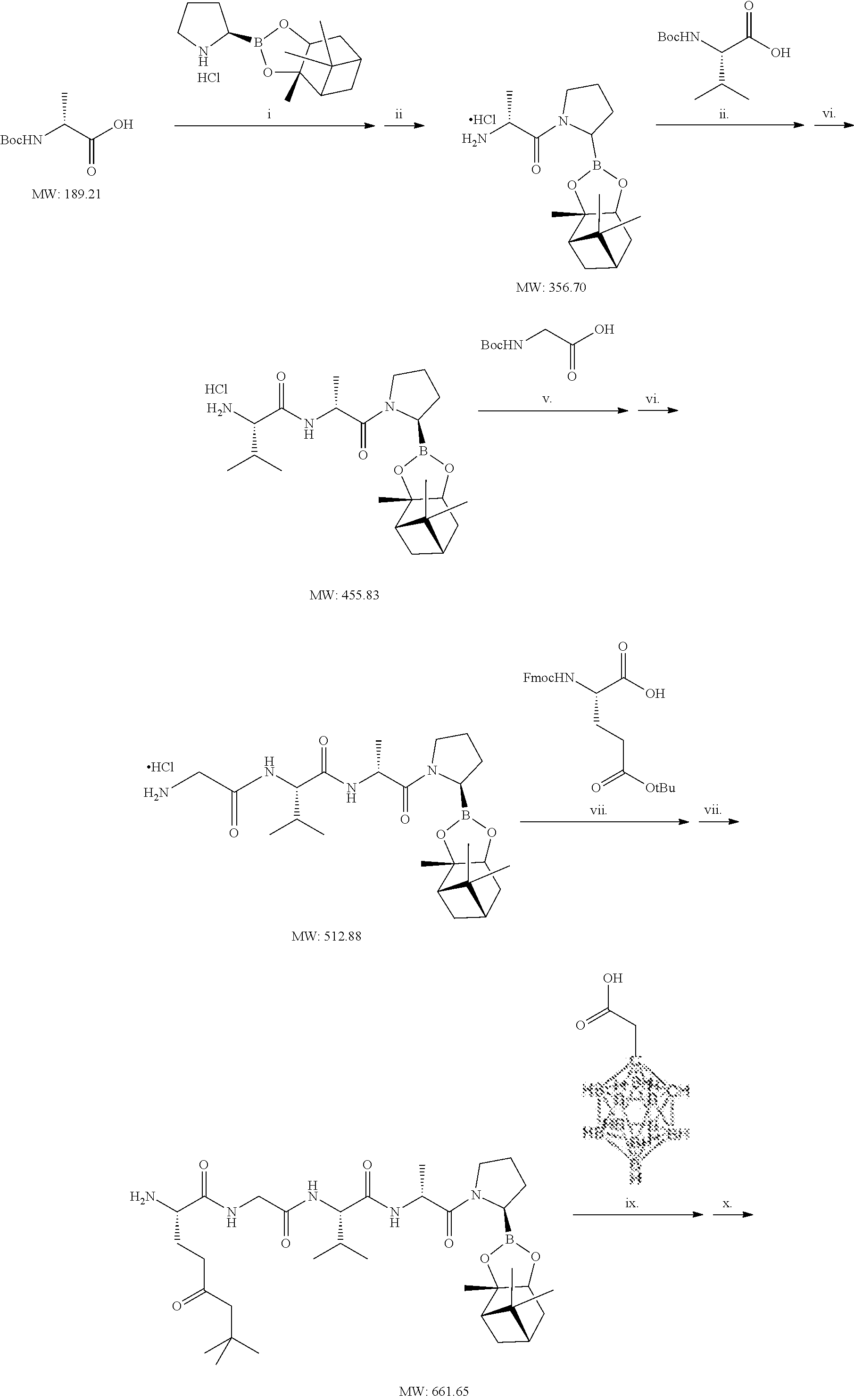

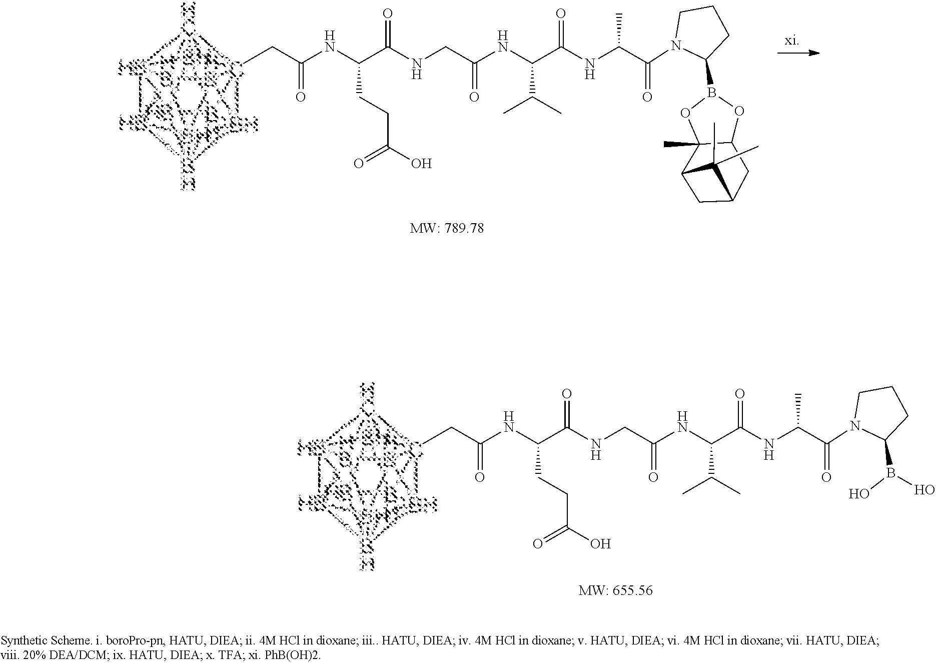

### Synthesis of H-Glu(OtBu)-Gly-Val-D-Ala-boroPro-Pn

To a stirred solution of N-Boc-Gly-OH (0.25 g, 1.43 mmol) in anhydrous DMF (7 mL) was added HATU (0.57 g, 1.5 mmol), Val-D-Ala-boroPro-pn·HCl (0.68 g, 1.5 mmol), and DIEA (0.55 mL) under ice-water bath cooling. The resulting mixture was stirred at room temperature for 2 hr and then condensed in vacuo. The residue was dissolved with DCM (60 mL), washed sequentially by 0.5N KHSO₄ (3×15 mL), aq. NaHCO₃ (3×15 mL), brine (15 mL). The organic phase was dried over anhydrous MgSO₄, filtered, and evaporated in vacuo to give N-Boc-Gly-Val-D-Ala-L-boroPro-pn which then added to a solution of 4N HCl in dioxane (10 mL) under ice-water cooling. The resulting mixture was stirred at room temperature for 2 hr and then condensed in vacuo. The residue was co-evaporated with dichloromethane (3×20 mL) in vacuo to completely dry. H-Gly-Val-D-Ala-boroPro-pn·HCl was thus obtained as a pale-yellow solid (0.8 g).

To a stirred solution of N-Fmoc-Glu(OtBu)-OH (0.57 g, 1.33 mmol) in anhydrous DMF (7 mL) was added HATU (0.53 g), H-Gly-Val-D-Ala-boroPro-pn·HCl (0.72 g), and DIEA (0.51 mL) under ice-water bath cooling. The resulting mixture was stirred at room temperature for 2 hr and then condensed in vacuo. The residue was dissolved with DCM (60 mL), washed sequentially by 0.5N KHSO₄ (3×15 mL), aq. NaHCO₃ (3×15 mL), brine (15 mL). The organic phase was dried over anhydrous MgSO4, filtered, and evaporated in vacuo to give N-Fmoc-Glu(OtBu)-Gly-Val-D-Ala-L-boroPro-pn which was then treated with 20% of DEA in DCM (7 mL). The resulting mixture was stirred at room temperature overnight, and then condensed in vacuo. The residue was co-evaporated with dichloromethane (3×15 mL) in vacuo to completely dry, then triturated with Hexane (50 mL), filtered, dried to give H-Glu(OtBu)-Gly-Val-D-Ala-boroPro-pn (0.9 g).

### Synthesis of Decaborane-Acetyl-Glu-Gly-Val-D-Ala-boroPro (Compound 7348)

To a stirred solution of o-decaborane-acetic acid (0.257 g, 1.27 mmol) in anhydrous DMF (6.5 mL) was added HATU (0.506 g), H-Glu(OtBu)-Gly-Val-D-Ala-boroPro-pn (0.9 g, 1.33 mmol), and DIEA (0.27 mL) under ice-water bath cooling. The resulting mixture was stirred at room temperature for 2 hr and then condensed in vacuo. The residue was dissolved with DCM (60 mL), washed sequentially by 0.5N KHSO₄ (3×15 mL), aq. NaHCO₃ (3×15 mL), brine (15 mL). The organic phase was dried over anhydrous MgSO₄, filtered, and evaporated in vacuo to give Decaborane-acetyl-Glu(OtBu)-Gly-Val-D-Ala-L-boroPro-pn which was then treated with 90% TFA in DCM (10 mL) under ice-water cooling. The resulting mixture was stirred at room temperature for 2.5 hr and then condensed in vacuo. The residue was co-evaporated with dichloromethane (3×20 mL) in vacuo to completely dry. Cold water (50 mL) and acetonitrile (20 mL) were added. The pH was adjusted to 1.5 with 5% HCl. Hexanes (100 mL) and phenylboronic acid (310 mg) were added. The resulting mixture was stirred at room temperature for 3 hr. The aqueous phase was separated, washed with hexanes (2×15 mL), concentrated in vacuo, and purified by semi-preparative HPLC, lyophilized to give the target product. LC-MS (ESI⁺) m/z (rel intensity): 1257.87 (100), 638.26 ([M−H₂O+H]⁺, 26), 620.61 (81); tr=9.07 min.

### Synthesis of Compound 6990

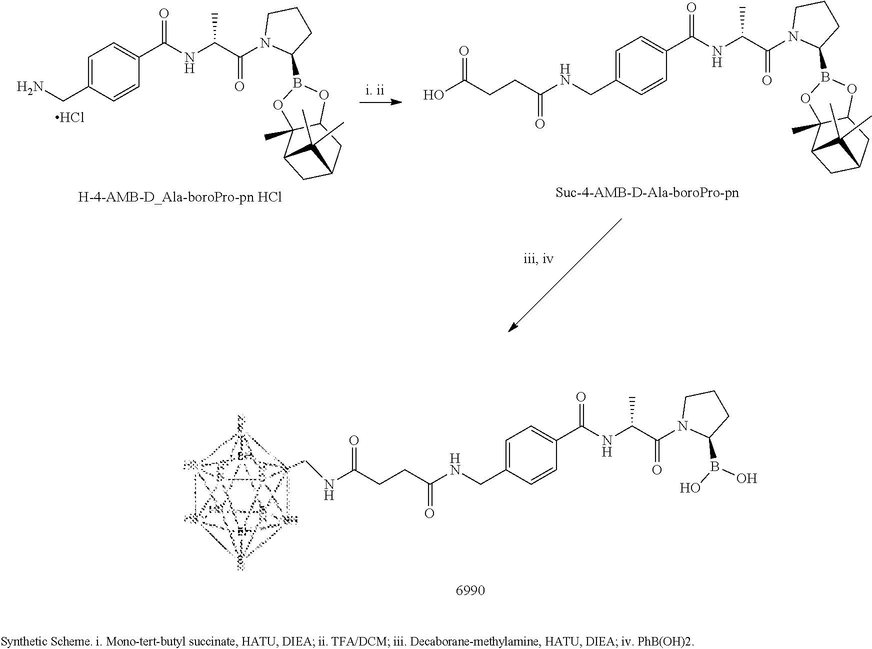

### Synthesis of Suc-4-AMB-D-Ala-boroPro-Pn

To a stirred solution of Mono-tert-butyl succinate (0.174 g, 1 mmol) in anhydrous DMF (5 mL) was added HATU (0.4 g), H-4-AMB-D-Ala-boroPro-pn·HCl (0.514 g, 1.05 mmol), and DIEA (0.38 mL) under ice-water bath cooling. The resulting mixture was stirred at room temperature for 2 hr and then condensed in vacuo. The residue was dissolved with DCM (50 mL), washed sequentially by 0.25N KHSO₄ (3×10 mL), aq. NaHCO₃ (3×10 mL), brine (10 mL). The organic phase was dried over anhydrous MgSO₄, filtered, and evaporated in vacuo, and then was treated with 75% TFA in DCM (10 mL) under ice-water cooling. The resulting mixture was stirred at room temperature for 2 hr and then condensed in vacuo. The residue was co-evaporated with dichloromethane (3×10 mL) in vacuo to completely dry to give Suc-4-AMB-D-Ala-boroPro-pn (0.9 g) as a yellow oil.

### Synthesis of Decaborane-Methylamino-Suc-AMB-D-Ana-boroPo (Compound 6990)

To a stirred solution of Suc-4-AMB-D-Ala-boroPro-pn (0.553 g, 1 mmol) in anhydrous DMF (5 mL) was added HATU (0.4 g), DIEA (0.38 mL) and Decaborane-methylamine·HCl (0.22 g) under ice-water bath cooling. The resulting mixture was stirred at room temperature for 2 hr and then condensed in vacuo. The residue was dissolved with DCM (50 mL), washed sequentially by 0.25N KHSO₄ (3×10 mL), aq. NaHCO₃ (3×10 mL), brine (3×10 mL). The organic phase was dried over anhydrous MgSO₄, filtered, and evaporated in vacuo. The residue was dissolved into water (40 mL) and acetonitrile (20 mL). The pH was adjusted to 1.5 with 5% HCl. Hexanes (80 mL) and phenylboronic acid (244 mg) were added. The resulting mixture was stirred at room temperature for 3 hr. The aqueous phase was separated, washed with hexanes (2×10 mL), concentrated in vacuo, and purified by semi-preparative HPLC, lyophilized to give the target product. LC-MS (ESI⁺, Instrumentation: Agilent 1290 HPLC/6460 Triple Quad LC/MS; column: Zorbax Eclipse Plus C18, 2.1×50 mm, 1.8 μm; HPLC method: Mobile Phase A: 0.1% TFA in water, Mobile Phase B: 0.08% TFA in ACN; Flow rate: 0.4 mL/min, Gradient: 0-1 min, 2% B: 5-6 min, 98% B) m/z (rel intensity): 1114.3 (20), 557.7 ([M−H₂O+H]⁺, 100), 272.4 (55); tr=3.674 min.

### Synthesis of Compound 7044

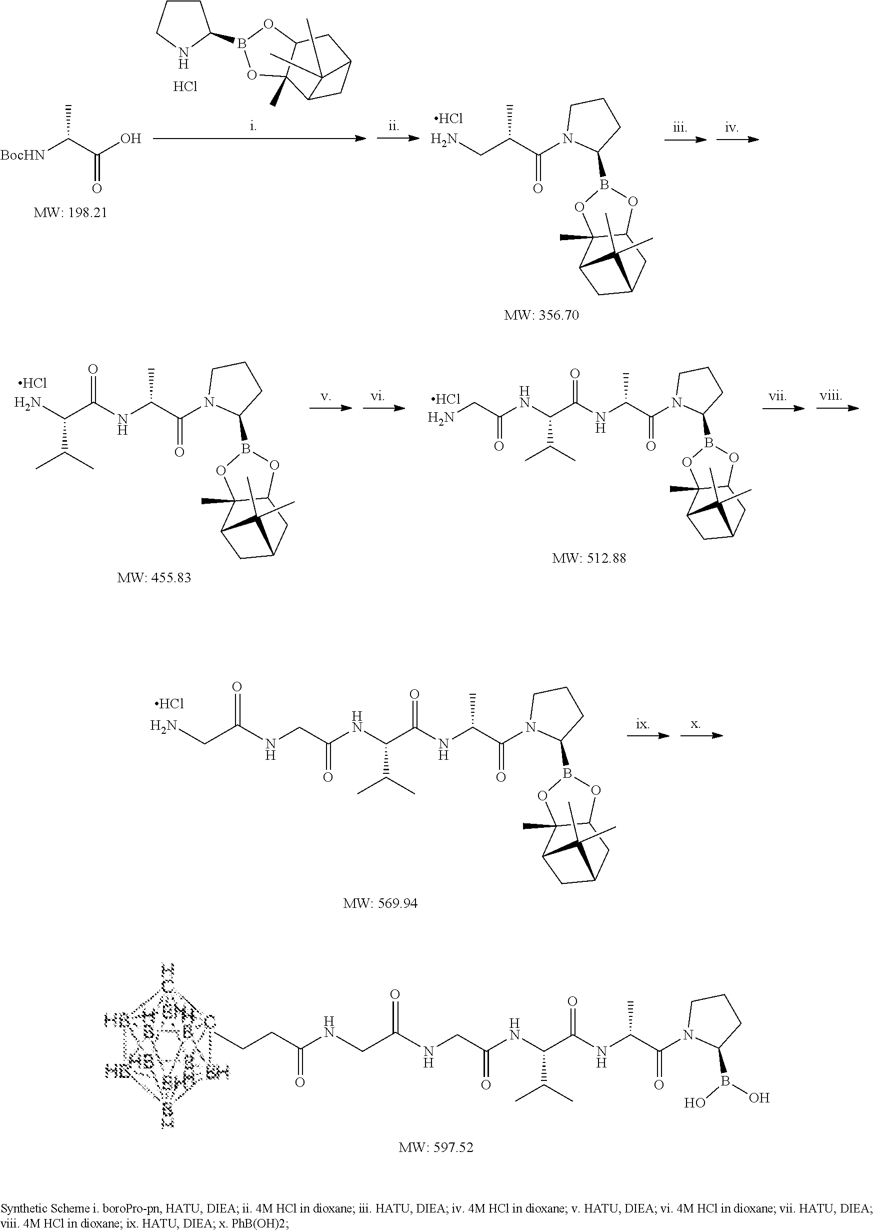

### Synthesis of H-Gly-Gly-Val-D-Ala-boroPro-Pn

To a stirred solution of N-Boc-Gly-OH (0.36 g, 2.04 mmol) in anhydrous DMF (20 mL) was added HATU (0.81 g), H-Gly-Val-D-Ala-boroPro-pn·HCl (1.1 g), and DIEA (0.78 mL) under ice-water bath cooling. The resulting mixture was stirred at room temperature for 2 hr and then condensed in vacuo. The residue was dissolved with EtOAc (100 mL), washed sequentially by 0.25N KHSO₄ (3×20 mL), aq. NaHCO₃ (3×20 mL), brine (3×20 mL). The organic phase was dried over anhydrous MgSO₄, filtered, and evaporated in vacuo to give N-Boc-Gly-Gly-Val-D-Ala-L-boroPro-pn which then added to a solution of 4N HCl in dioxane (30 mL) under ice-water cooling. The resulting mixture was stirred at room temperature for 2 hr and then condensed in vacuo. The residue was co-evaporated with dichloromethane (3×20 mL) in vacuo to completely dry. H-Gly-Gly-Val-D-Ala-boroPro-pn·HCl was thus obtained as a pale-yellow solid (1.23 g).

### Synthesis of Decaborane-Propionyl-Gly-Gly-Val-D-Ala-boroPro (Compound 7044)

To a stirred solution of o-decaborane-propionic acid (0.21 g, 0.97 mmol) in anhydrous DMF (10 mL) was added HATU (0.39 g), H-Gly-Gly-Val-D-Ala-boroPro-pn·HCl (0.61 g), and DIEA (0.37 mL) under ice-water bath cooling. The resulting mixture was stirred at room temperature for 2 hr and then condensed in vacuo. The residue was dissolved with DCM (50 mL), washed sequentially by 0.25N KHSO₄ (3×10 mL), aq. NaHCO₃ (3×10 mL), brine (3×10 mL). The organic phase was dried over anhydrous MgSO₄, filtered, and evaporated in vacuo. The residue was suspended in water (40 mL) and acetonitrile (20 mL). The pH was adjusted to 1.5 with 5% HCl. Hexanes (80 mL) and phenylboronic acid (240 mg) were added. The resulting mixture was stirred at room temperature for 3 hr. The aqueous phase was separated, washed with hexanes (2×10 mL), concentrated in vacuo, and purified by semi-preparative HPLC, lyophilized to give the target product. LC-MS (ESI⁺, Instrumentation: Agilent 1290 HPLC/6460 Triple Quad LC/MS; column: Zorbax Eclipse Plus C18, 2.1×50 mm, 1.8 μm; HPLC method: Mobile Phase A: 0.1% TFA in water, Mobile Phase B: 0.08% TFA in ACN; Flow rate: 0.4 mL/min, Gradient: 0-1 min, 2% B; 5-6 min, 98% B) m/z (rel intensity): 1160.3 (26), 580.7 ([M−H₂O+H]⁺, 100); tr=3.696 min.

### Synthesis of Compound 7389

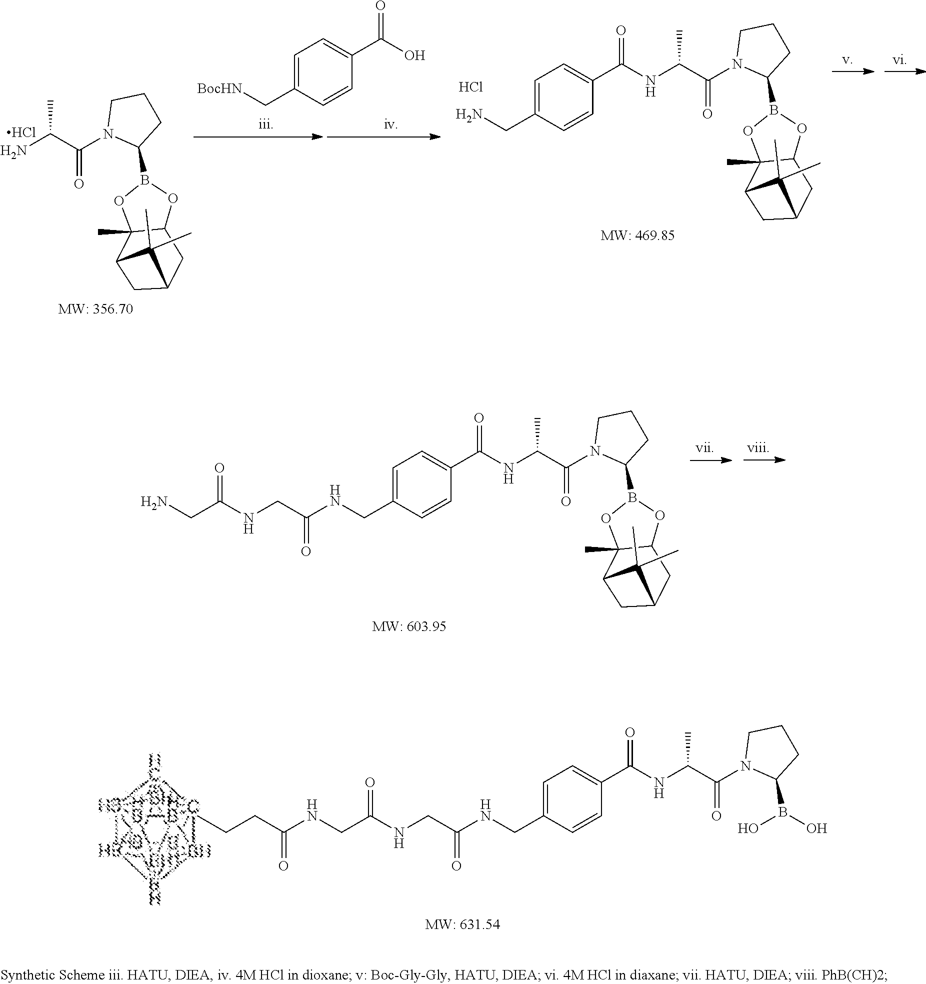

### Synthesis of H-Gly-Gly-AMB-D-Ala-boroPro-pn·HCl

To a stirred solution of N-Boc-Gly-Gly-OH (1.26 g, 5.44 mmol) in anhydrous DMF (50 mL) was added HATU (2.17 g), DIEA (2.1 mL) and H-AMB-D-Ala-boroPro-pn·HCl (2.8 g) under ice-water bath cooling. The resulting mixture was stirred at room temperature for 2 hr and then condensed in vacuo. The residue was dissolved with ethyl acetate (150 mL), washed sequentially by 0.25 N KHSO₄ (3×30 mL), aq. NaHCO₃ (3×30 mL), brine (3×30 mL). The organic phase was dried over anhydrous MgSO₄, filtered, and evaporated in vacuo, purified by silica gel flash chromatography eluted with MeOH/DCM, and was then added to a solution of 4N HCl in dioxane (25 mL) under ice-water cooling. The resulting mixture was stirred at room temperature for 2 hr and then condensed in vacuo. The residue was co-evaporated with dichloromethane (3×30 mL) in vacuo to completely dry. H-Gly-Gly-AMB-D-Ala-boroPro-pn·HCl was thus obtained as a pale-yellow solid (1.0 g).

### Synthesis of Decaborane-Propionyl-Gly-Gly-AMB-D-Ala-boroPro (Compound 7389)

To a stirred solution of o-decaborane-propionic acid (340 mg, 1.57 mmol) in anhydrous DMF (15 mL) was added HATU (629 mg), DIEA (0.6 mL) and H-Gly-Gly-AMB-D-Ala-boroPro-pn·HCl (1.0 g) under ice-water bath cooling. The resulting mixture was stirred at room temperature for 2 hr and then condensed in vacuo. The residue was dissolved with ethyl acetate (50 mL), washed sequentially by 0.25 N KHSO₄ (3×10 mL), aq. NaHCO₃ (3×10 mL), brine (3×10 mL). The organic phase was dried over anhydrous MgSO₄, filtered, and evaporated in vacuo. The residue was dissolved in water (40 mL) and acetonitrile (20 mL). The pH was adjusted to 1.5 with 5% HCl. Hexanes (80 mL) and phenylboronic acid (383 mg) were added. The resulting mixture was stirred at room temperature for 3 hr. The aqueous phase was separated, washed with hexanes (2×20 mL), concentrated in vacuo, and purified by semi-preparative HPLC, lyophilized to give the target product. LC-MS (ESI⁺, Instrumentation: Agilent 1290 HPLC/6460 Triple Quad LC/MS; column: Poroshell 120 CS-C18, 2.1×50 mm, 2.7 μm; HPLC method: Mobile Phase A: 0.1% TFA in water, Mobile Phase B: 0.08% TFA in ACN; Flow rate: 0.5 mL/min, Gradient: 0-1 min, 5% B; 5-6 min, 98% B) m/z (rel intensity): 1160.3 (26), 580.7 ([M−H₂O+H]⁺, 100); tr=3.696 min.

### Synthesis of Compound 7036

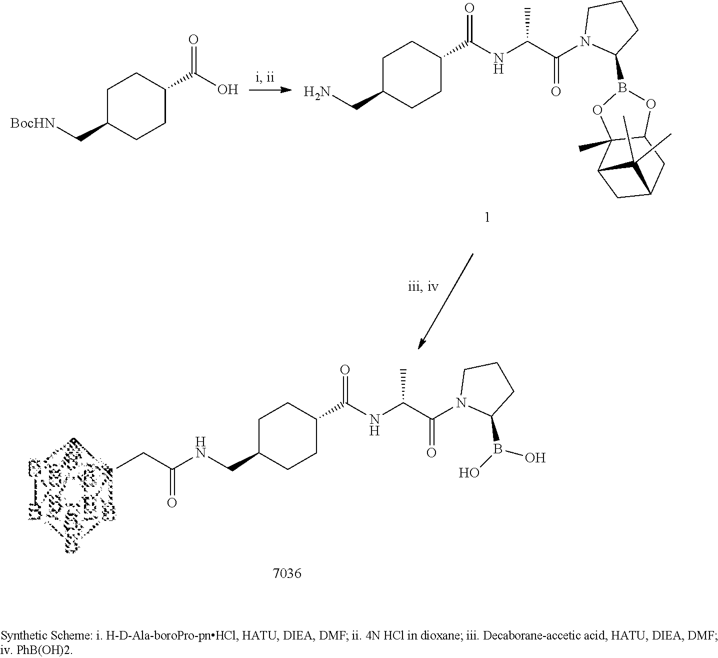

### Synthesis of Decaborane-Acetyl-TXA-D-Ala-boroPro (Compound 7036)

To a stirred solution of o-decaborane-acetic acid (155 mg, 0.77 mmol) in anhydrous DMF (10 mL) was added HATU (307 mg), DIEA (0.3 mL) and H-TXA-D-Ala-boroPro-pn·HCl (400 mg) under ice-water bath cooling. The resulting mixture was stirred at room temperature for 2 hr and then condensed in vacuo. The residue was dissolved with ethyl acetate (50 mL), washed sequentially by 0.25 N KHSO₄ (3×10 mL), aq. NaHCO₃ (3×10 mL), brine (3×10 mL). The organic phase was dried over anhydrous MgSO₄, filtered, and evaporated in vacuo to give Decaborane-acetyl-TXA-D-Ala-L-boroPro-pn. Water (40 mL) and acetonitrile (20 mL) were added. The pH was adjusted to 1.5 with 5% HCl. Hexanes (80 mL) and phenylboronic acid (188 mg) were added. The resulting mixture was stirred for 3 hr at room temperature. The aqueous phase was separated, washed with hexane (2×10 mL), concentrated in vacuo, and purified by semi-preparative HPLC, lyophilized to give the target product. LC-MS (ESI⁺, Instrumentation: Agilent 1290 HPLC/6460 Triple Quad LC/MS; column: Zorbax Eclipse Plus C18, 2.1×50 mm, 1.8 μm; HPLC method: Mobile Phase A: 0.1% TFA in water, Mobile Phase B: 0.08% TFA in ACN; Flow rate: 0.4 mL/min, Gradient: 0-3 min. 2% B; 12-15 min, 98% B) m/z (rel intensity): 492.4 ([M−H₂O+H]⁺, 100); tr=8.252 min.

### Synthesis of Compound 7042

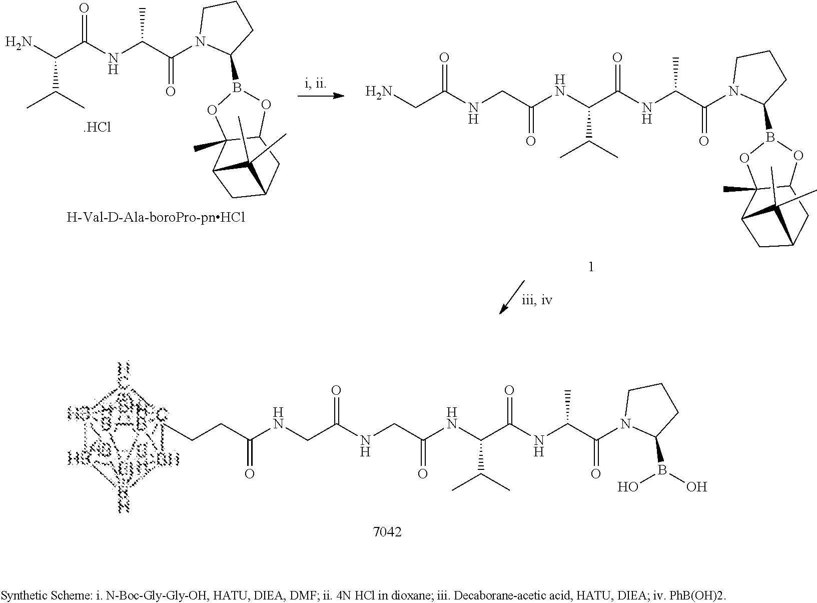

### Synthesis of Decaborane-Acetyl-Gly-Gly-Val-Ala-boroPro (Compound 7042)

To a stirred solution of o-decaborane-acetic acid (0.237 g, 1.17 mmol) in anhydrous DMF (1.2 mL) and DCM (15 mL) was added HATU (0.468 g), H-Gly-Gly-Val-D-Ala-boroPro-pn·HCl (0.7 g), and DIEA (0.45 mL) under ice-water bath cooling. The resulting mixture was stirred at room temperature for 2 hr and then condensed in vacuo. The residue was dissolved with DCM (45 mL), washed sequentially by 0.5N KHSO₄ (3×15 mL), aq. NaHCO₃ (3×15 mL), brine (15 mL). The organic phase was dried over anhydrous MgSO4, filtered, and evaporated in vacuo. The residue was suspended in water (50 mL) and acetonitrile (20 mL). The pH was adjusted to 1.5 with 5% HCl. Hexanes (100 mL) and phenylboronic acid (285 mg) were added. The resulting mixture was stirred at room temperature for 3 hr. The aqueous phase was separated, washed with hexanes (2×15 mL), concentrated in vacuo, and purified by semi-preparative HPLC, lyophilized to give the target product. LC-MS (ESI⁺) m/z (rel intensity): 1132.05 (73), 566.71 ([M−H₂O+H]⁺, 100); tr=9.15 min.

### Synthesis of Compound 7519

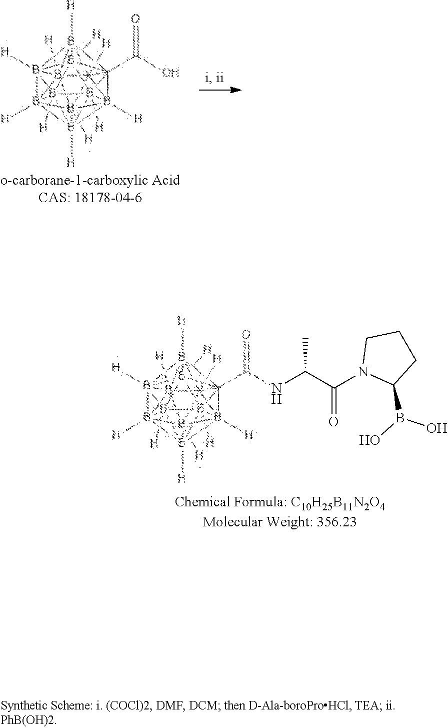

### Synthesis of N-(Decaborane-Carbonyl)-D-Ala-boroPro-Pn

A dichloromethane solution (10.0 mL) of o-carborane-1-carboxylic acid (101 mg, 0.5 mmol) and DMF (0.05 mL) was cooled to 0° C., to which was slowly added oxalyl chloride (0.13 mL). The resulting solution was stirred for 1 h at room temperature, followed by removal of the solvent and excess oxalyl chloride in vacuo. The residue was co-evaporated with dichloromethane (3×10 mL) in vacuo, and then was re-dissolved in dichloromethane (5.0 m) and cooled to 0° C., to which was added D-Ala-boroPro-pn·HCl (190 mg) and triethylamine (0.42 mL). The reaction solution was stirred at room temperature overnight and then quenched with water (5 mL). The organic layer was separated, and the aqueous layer was extracted with dichloromethane (2×10 mL). The organic solutions were combined, concentrated in vacuo, and purified by semi-preparative HPLC, lyophilized to give N-(decaborane-carbonyl)-D-Ala-boroPro-pn. LC-MS (ESI⁺) m/z (rel intensity): 491.78 ([M+H]⁺, 100), 339.76 (60); tr=11.33 min.

### Synthesis of N-(Decaborane-Carbonyl)-D-Ala-boroPro-Pn (Compound 7519)

N-(decaborane-carbonyl)-D-Ala-boroPro-pn obtained above was dissolved into water (10 mL) and acetonitrile (5 mL). The pH was adjusted to 2 with 1N TFA. MTBE (30 mL) and phenylboronic acid (66 mg) were added. The resulting mixture was stirred at room temperature overnight, and then concentrated in vacuo, purified by semi-preparative HPLC, lyophilized to give the target product. LC-MS (ESI⁺) m/z (rel intensity): 1038.30 (100), 718.91 (25), 667.98 (36), 339.71 ([M−H₂O+H]⁺, 98); tr=9.64 min.

### Synthesis of Compound 7037 (aka 7281)

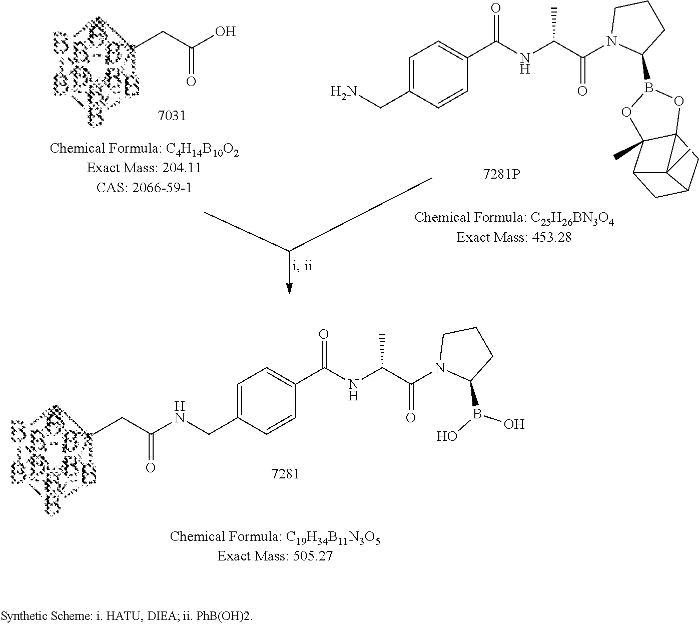

### Synthesis of Decaborane-Acetyl-AMB-D-Ala-boroPro (Compound 7037) G-m,

To a stirred solution of o-decaborane-acetic acid (51 mg, 0.25 mmol) in anhydrous DMF (3 mL) was added HATU (100 mg), DIEA (0.2 mL) and H-AMB-D-Ala-boroPro-pn·HCl (130 mg) under ice-water bath cooling. The resulting mixture was stirred at room temperature for 2 hr and then condensed in vacuo. The residue was dissolved with ethyl acetate (30 mL), washed sequentially by 0.1 N KHSO₄ (3×10 mL), aq. NaHCO₃ (3×10 mL), brine (10 mL). The organic phase was dried over anhydrous MgSO₄, filtered, and evaporated in vacuo to give Decaborane-acetyl-AMB-D-Ala-L-boroPro-pn. Water (10 mL) and acetonitrile (5 mL) were added. The pH was adjusted to 2 with 1N TFA. MTBE (25 mL) and phenylboronic acid (33 mg) were added. The resulting mixture was stirred at room temperature overnight. The aqueous phase was separated, washed with MTBE (2×10 mL), concentrated in vacuo, and purified by semi-preparative HPLC, lyophilized to give the target product. LC-MS (ESI⁺, with ZORBAX Eclipse Plus C18 RP-HPLC column (4.6×50 mm, 1.8 μm), eluent gradient 10% B for the first 3 min, then from 10% to 98% B over 7 min, which was maintained for the next 5 min) m/z (rel intensity): 972.87 (66), 486.58 ([M−H₂O+H]⁺, 100); tr=8.89 min. ¹H NMR (D₂O/CAN-d3): δ 1.50 (d, J=7.2 Hz, 3H), 1.75-1.79 (m, 1H), 1.80-2.20 (m, 14H), 3.07-3.10 (m, 1H), 3.30 (s, 2H), 3.71-3.74 (m, 2H), 4.50 (d, J=4.8 Hz, 2H), 7.52 (d, J=8.4 Hz, 2H), 7.92 (d, J=7.8 Hz, 2H), 8.67 (s, 1H).

### Synthesis of Compound 7041

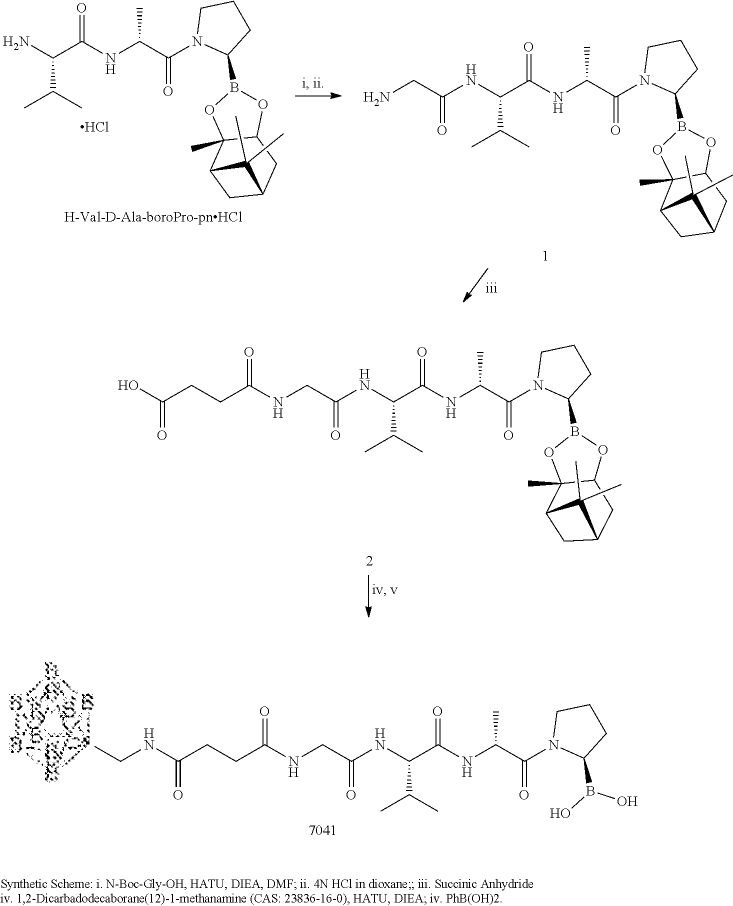

### Synthesis of Suc-Gly-Val-D-Ala-boroPro-PN

To a stirred solution of H-Gly-Val-D-Ala-boroPro-pn·HCl (0.8 g, 1.56 mmol) in anhydrous DCM (80 mL) was added DIEA (1.03 mL) under ice-water bath cooling. A solution of succinic anhydride (468 mg) in DCM (20 mL) was then added dropwise. The resulting mixture was stirred at 0° C. for 1 hr, and then at room temperature for 2 hr. The mixture was washed sequentially by 0.25N KHSO₄ (3×10 mL), aq. NaHCO₃ (3×10 mL), brine (10 mL). The organic phase was dried over anhydrous MgSO₄, filtered, and evaporated in vacuo to give Suc-Gly-Val-D-Ala-boroPro-pn (0.8 g) as a pale-white solid.

### Synthesis of Decaborane-Methylamino-Suc-Gly-Val-D-Ala-boroPro (Compound 7041)

To a stirred solution of Suc-Gly-Val-D-Ala-boroPro-pn (0.8 g, 1.39 mmol) in anhydrous DMF (15 mL) was added HATU (0.555 mg), DIEA (0.5 mL) and Decaborane-methylamine·HCl (0.253 g) under ice-water bath cooling. The resulting mixture was stirred at room temperature for 2 hr and then condensed in vacuo. The residue was dissolved with EtOAc (50 mL), washed sequentially by 0.25N KHSO₄ (3×10 mL), aq. NaHCO₃ (3×10 mL), brine (3×10 mL). The organic phase was dried over anhydrous MgSO₄, filtered, and evaporated in vacuo. The residue was dissolved into water (40 mL) and acetonitrile (20 mL). The pH was adjusted to 1.5 with 5% HCl. Hexanes (80 mL) and phenylboronic acid (322 mg) were added. The resulting mixture was stirred at room temperature for 3 hr. The aqueous phase was separated, washed with hexanes (2×10 mL), concentrated in vacuo, and purified by semi-preparative HPLC, lyophilized to give the target product. LC-MS (ESI⁺, Instrumentation: Agilent 1290 HPLC/6460 Triple Quad LC/MS; column: Poroshell 120 CS-C18, 2.1×50 mm, 2.7 μm; HPLC method: Mobile Phase A: 0.1% TFA in water, Mobile Phase B: 0.08% TFA in ACN; Flow rate: 0.5 mL/min, Gradient: 0-1 min, 5% B; 5-6 min, 98% B) m/z (rel intensity): 1160.3 (20), 580.6 ([M−H₂O+H]⁺, 100); tr=3.406 min.

### Synthesis of Compound 7311

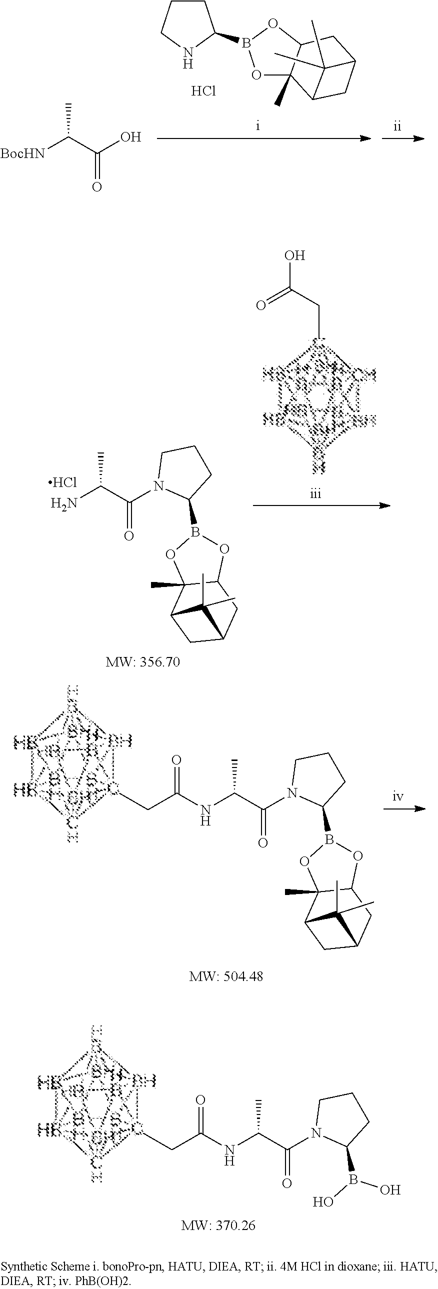

### Synthesis of Decaborane-Acetyl-D-Ala-boroPro (Compound 7311)

To a stirred solution of o-decaborane-acetic acid (193 mg, 0.95 mmol) in anhydrous DMF (1 mL) and DCM (10 mL) was added HATU (380 mg), DIEA (0.36 mL) and H-D-Ala-boroPro-pn·HCl (357 mg) under ice-water bath cooling. The resulting mixture was stirred at room temperature for 2 hr and then condensed in vacuo. The residue was dissolved with DCM (30 mL), washed sequentially by 0.5 N KHSO₄ (3×10 mL), aq. NaHCO₃ (3×10 mL), brine (10 mL). The organic phase was dried over anhydrous MgSO₄, filtered, and evaporated in vacuo to give Decaborane-acetyl-D-Ala-L-boroPro-pn. Water (40 mL) and acetonitrile (16 mL) were added. The pH was adjusted to 1.5 with 5% HCL. Hexanes (80 mL) and phenylboronic acid (232 mg) were added. The resulting mixture was stirred at room temperature for 3 hr. The aqueous phase was separated, washed with hexanes (2×10 mL), concentrated in vacuo, and purified by semi-preparative HPLC, lyophilized to give the target product. LC-MS (ESI⁺, eluent gradient 5% B for the first 3 min, then from 5% to 98% B over 12 min, which was maintained for the next 5 min) m/z (rel intensity): 1080.08 (83), 705.81 (100), 352.73 ([M−H₂O+H]⁺, 98); tr=9.39 min.

### Example 2: In Vitro Assay

Biological Materials: For the in vitro IC50 determination assays, recombinant human DPPIV, DPP9, FAP, and PREP were purchased from R&D Systems, and DPP8 was from Biomol International. Buffer systems used were A (25 mM Tris, pH 8.0), B (50 mM Tris, pH 7.5), C (50 mM Tris, 140 mM NaCl, pH 7.5), D (25 mM Tris, 250 mM NaCl, pH 7.5), and E (20 mM Tris, 20 mM KCl, pH 7.4). Fluorogenic substrates were Gly-Pro-AMC, Z-GlyPro-AMC, or Suc-Gly-Pro-AMC purchased from Bachem or an N terminally blocked FAP specific substrate. The cell culture medium was RPMI 1640 without phenol red and supplemented with 2 mM L glutamine, 10 mM HEPES, 1 mM sodium pyruvate, 4500 mg/L glucose, 100 IU/mL penicillin, and 100 μg/mL streptomycin. Substrate Specificity Assay. Peptide libraries (0.21 mM) were incubated for 24 h with 1 nM FAP in buffer E at 37° C. The reaction was quenched by the addition of 1.2 N HCL. The samples were analyzed by reverse-phase HPLC-MS on a Thermo Finnigan LCQ Duo, quantifying the peaks in the resulting base peak chromatograms. Relative cleavage values were determined by comparing the post quench abundance of intact peptides to those in the initial library.

In Vitro Enzyme IC₅₀ Assays. Enzymatic activity of DPPIV, DPP8, DPP9, FAP, and PREP was measured at 25° C. on a Molecular Devices M2e multidetection microtiter plate reader, monitoring the fluorescence at an excitation wavelength of 380 nm and an emission wavelength of 460 num. The substrate was either H-Gly-Pro-AMC for the DPPIV, DPP8, and DPP9 assays or Z-Gly-Pro-AMC for the FAP and PREP assays. The reaction mixture contained 25 μM substrate, enzyme, buffer A (DPPIV and DPP9), buffer B (DPP8), buffer C (FAP), or buffer D (PREP) and a suitable amount of inhibitor (ranging between 10-4 and 10-11 M) in a total volume of 210 μL. The final enzyme concentrations were 0.1, 0.8, 0.4, 1.2, and 0.6 nM for DPPIV, DPP8, DPP9, FAP, and PREP, respectively. The IC₅₀ value is defined as the concentration of inhibitor required to reduce the enzyme activity by 50% after a 10 min preincubation with the enzyme at 25° C. prior to addition of the substrate. Inhibitor stock solutions (100 mM) were prepared in either a pH 2.0 HCl solution for compounds 1 and 20 or DMSO. Those prepared in pH 2.0 solution were preincubated at 25° C. for 4 h prior to dilution. Immediately prior to the commencement of the experiment, the 100 mM stocks were further diluted to 10-3 M in the appropriate assay buffer, from which 1:10 serial dilutions were prepared. All inhibitors were tested in triplicate. Results are shown in Table 1.

TABLE 1
In vitro Inhibition of rhFAP (IC₅₀)

| Com- | | IC₅₀ |
|---|---|---|
| pound | | |
| No. | Structure | (μM) |
| 7349 | 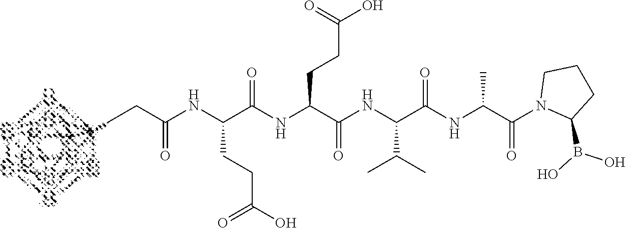 [SMILES: CCC(=O)N[C@@H](CCC(=O)O)C(=O)N[C@@H](CCC(=O)O)C(=O)N[C@H](C(=O)N[C@H](C)C(=O)N1CCC[C@H]1B(O)O)C(C)C] | 0.00196 |
| 7388 | 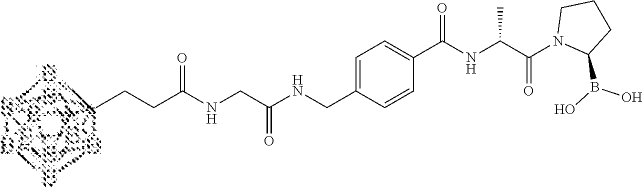 [SMILES: CCCC(=O)NCC(=O)NCc1ccc(C(=O)N[C@H](C)C(=O)N2CCC[C@H]2B(O)O)cc1] | 0.0028 |
| 7034 | 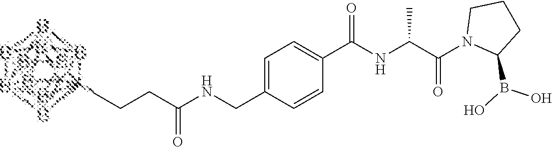 [SMILES: CCCC(=O)NCc1ccc(C(=O)N[C@H](C)C(=O)N2CCC[C@H]2B(O)O)cc1] | 0.0029 |
| 7350 | 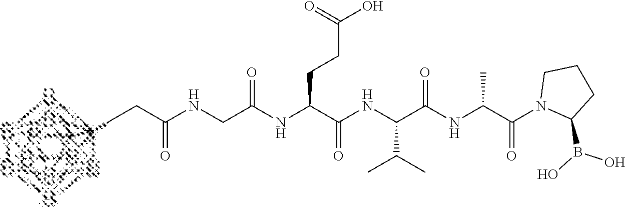 [SMILES: CCC(=O)NCC(=O)N[C@@H](CCC(=O)O)C(=O)N[C@H](C(=O)N[C@H](C)C(=O)N1CCC[C@H]1B(O)O)C(C)C] | 0.00611 |
| 7412 | 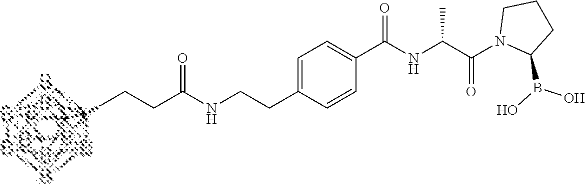 [SMILES: CCCC(=O)NCCc1ccc(C(=O)N[C@H](C)C(=O)N2CCC[C@H]2B(O)O)cc1] | 0.0062 |
| 7282 | 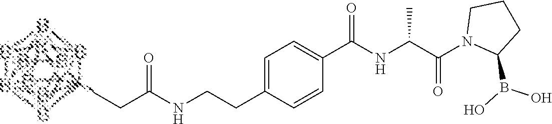 [SMILES: CCC(=O)NCCc1ccc(C(=O)N[C@H](C)C(=O)N2CCC[C@H]2B(O)O)cc1] | 0.009 |
| 7430B | 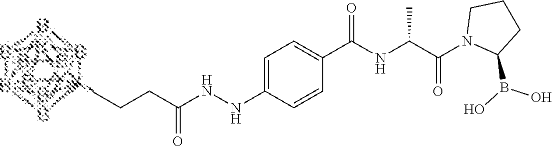 [SMILES: CCCC(=O)NNc1ccc(C(=O)N[C@H](C)C(=O)N2CCC[C@H]2B(O)O)cc1] | 0.009 |
| 7035 | 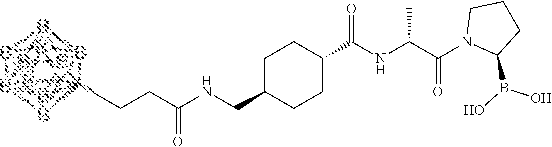 [SMILES: CCCC(=O)NC[C@H]1CC[C@H](C(=O)N[C@H](C)C(=O)N2CCC[C@H]2B(O)O)CC1] | 0.01325 |
| 7348 | 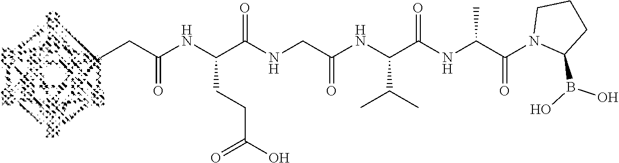 [SMILES: CCC(=O)N[C@@H](CCC(=O)O)C(=O)NCC(=O)N[C@H](C(=O)N[C@H](C)C(=O)N1CCC[C@H]1B(O)O)C(C)C] | 0.01558 |
| 6990 | 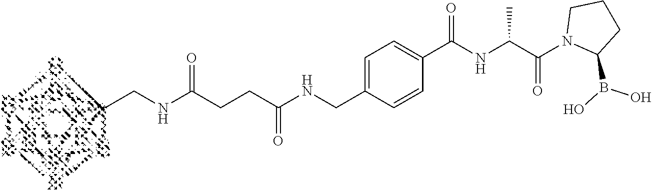 [SMILES: CCNC(=O)CCC(=O)NCc1ccc(C(=O)N[C@H](C)C(=O)N2CCC[C@H]2B(O)O)cc1] | 0.02717 |
| 7044 | 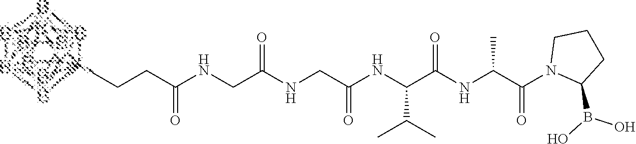 [SMILES: CCCC(=O)NCC(=O)NCC(=O)N[C@H](C(=O)N[C@H](C)C(=O)N1CCC[C@H]1B(O)O)C(C)C] | 0.03565 |
| 7389 | 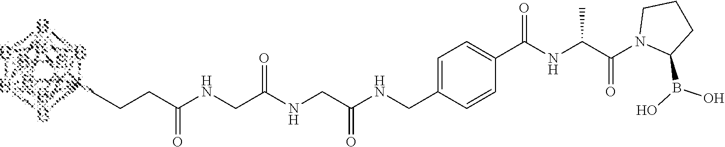 [SMILES: CCCC(=O)NCC(=O)NCC(=O)NCc1ccc(C(=O)N[C@H](C)C(=O)N2CCC[C@H]2B(O)O)cc1] | 0.03663 |
| 7036 | 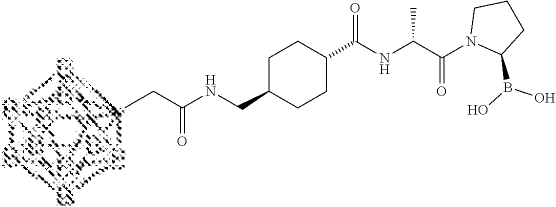 [SMILES: CCC(=O)NC[C@H]1CC[C@H](C(=O)N[C@H](C)C(=O)N2CCC[C@H]2B(O)O)CC1] | 0.03766 |
| 7042 | 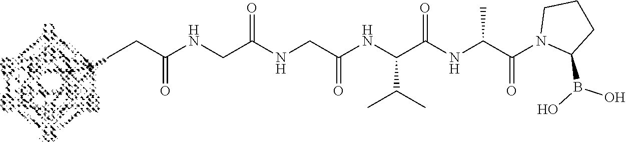 [SMILES: CCC(=O)NCC(=O)NCC(=O)N[C@H](C(=O)N[C@H](C)C(=O)N1CCC[C@H]1B(O)O)C(C)C] | 0.0488 |
| 7519 | 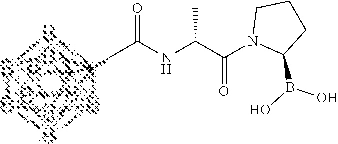 [SMILES: CC(=O)N[C@H](C)C(=O)N1CCC[C@H]1B(O)O] | 0.07708 |
| 7037 | 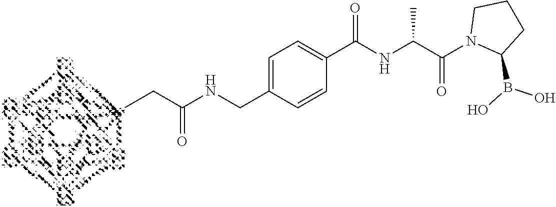 [SMILES: CCC(=O)NCc1ccc(C(=O)N[C@H](C)C(=O)N2CCC[C@H]2B(O)O)cc1] | 0.08049 |
| 7041 | 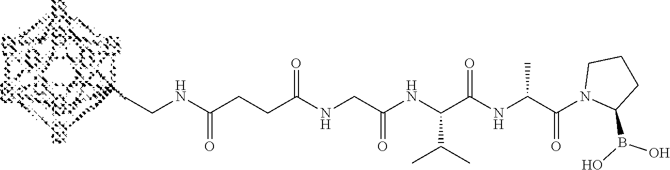 [SMILES: CCNC(=O)CCC(=O)NCC(=O)N[C@H](C(=O)N[C@H](C)C(=O)N1CCC[C@H]1B(O)O)C(C)C] | 0.0929 |
| 7311 | 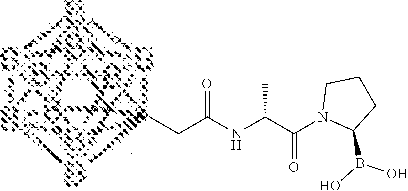 [SMILES: CCC(=O)N[C@H](C)C(=O)N1CCC[C@H]1B(O)O] | 0.0952 |
| 7429B | 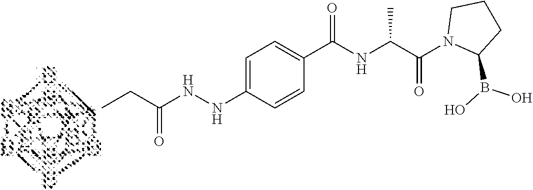 [SMILES: CCC(=O)NNc1ccc(C(=O)N[C@H](C)C(=O)N2CCC[C@H]2B(O)O)cc1] | 0.2184 |
| 7509 | 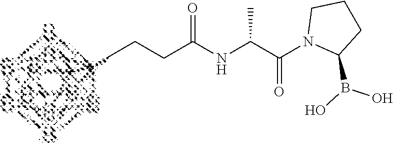 [SMILES: CCCC(=O)N[C@H](C)C(=O)N1CCC[C@H]1B(O)O] | 0.683 |
| 7312 | 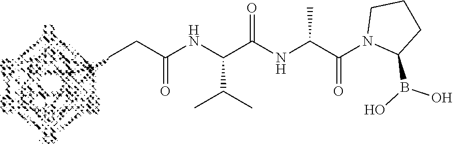 [SMILES: CCC(=O)N[C@H](C(=O)N[C@H](C)C(=O)N1CCC[C@H]1B(O)O)C(C)C] | 0.7377 |
| 7313 | 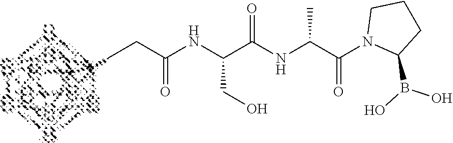 [SMILES: CCC(=O)N[C@@H](CO)C(=O)N[C@H](C)C(=O)N1CCC[C@H]1B(O)O] | 1.263 |
| 7633 | 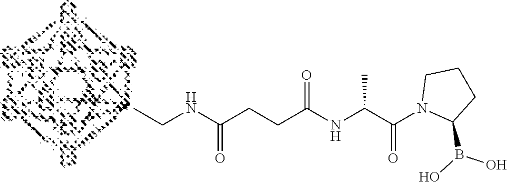 [SMILES: CCNC(=O)CCC(=O)N[C@H](C)C(=O)N1CCC[C@H]1B(O)O] | |
| 6988 | 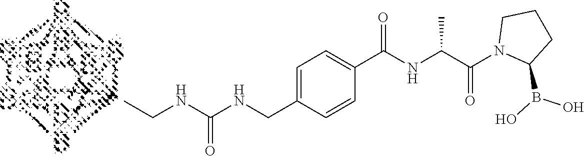 [SMILES: CCNC(=O)NCc1ccc(C(=O)N[C@H](C)C(=O)N2CCC[C@H]2B(O)O)cc1] | |
| 7519B | 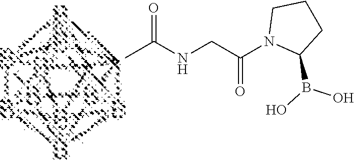 | |
| 7085 | 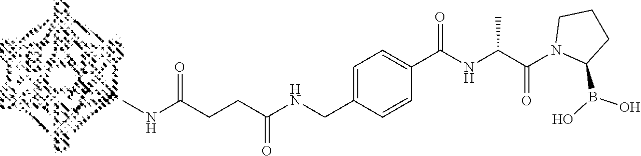 [SMILES: CNC(=O)CCC(=O)NCc1ccc(C(=O)N[C@H](C)C(=O)N2CCC[C@H]2B(O)O)cc1] | |
| 7644 | 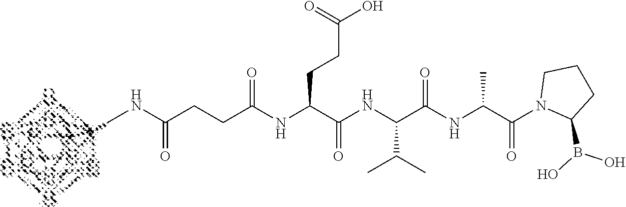 [SMILES: CNC(=O)CCC(=O)N[C@@H](CCC(=O)O)C(=O)N[C@H](C(=O)N[C@H](C)C(=O)N1CCC[C@H]1B(O)O)C(C)C] | |
| 7349T | 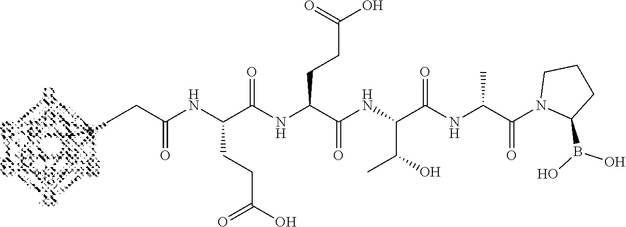 [SMILES: CCC(=O)N[C@@H](CCC(=O)O)C(=O)N[C@@H](CCC(=O)O)C(=O)N[C@H](C(=O)N[C@H](C)C(=O)N1CCC[C@H]1B(O)O)[C@@H](C)O] | |
| 7350T | 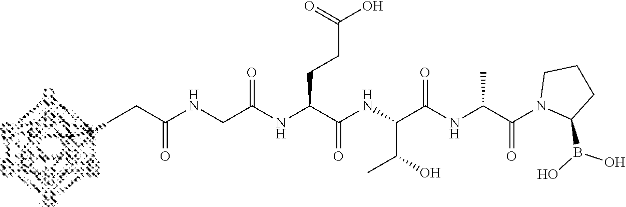 [SMILES: CCC(=O)NCC(=O)N[C@@H](CCC(=O)O)C(=O)N[C@H](C(=O)N[C@H](C)C(=O)N1CCC[C@H]1B(O)O)[C@@H](C)O] | |
| 7430 | 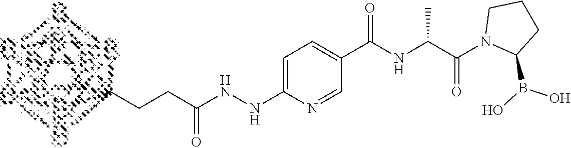 [SMILES: CCCC(=O)NNc1ccc(C(=O)N[C@H](C)C(=O)N2CCC[C@H]2B(O)O)cn1] | |
| 7429 | 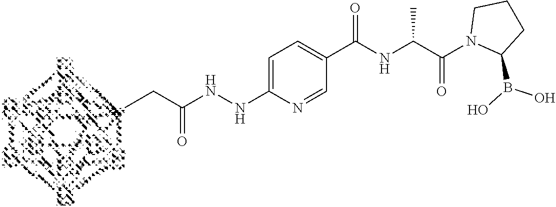 [SMILES: CCC(=O)NNc1ccc(C(=O)N[C@H](C)C(=O)N2CCC[C@H]2B(O)O)cn1] | |

### EQUIVALENTS

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

1. A composition for delivering neutron capture agents to cells expressing fibroblast activation protein (FAP), wherein the composition comprises a FAP binding moiety (FAP-bm) associated (covalently or non-covalently) with a neutron capture agent.

2-4. (canceled)

5. A compound of formula I: 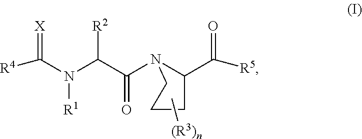
or a pharmaceutically acceptable salt thereof, wherein
X is O or S;
R¹ is H or (C₁-C₆) alkyl;
R² is H or (C₁-C₆) alkyl;
R³ is halogen, nitro, cyano, amino, acylamino, amido, hydroxyl, alkoxy, acyloxy, thiol, alkylthio, alkyl, aralkyl, heteroaralkyl, alkenyl, alkynyl, carbocyclyl, heterocyclyl, aryl, or heteroaryl;
n is an integer selected from 0 to 7;
R⁴ is amino, alkoxy, acyloxy, alkyl, aralkyl, heteroaralkyl, alkenyl, alkynyl, carbocyclyl, heterocyclyl, aryl, or heteroaryl; and
R⁵ is a moiety comprising at least one boron atom.

6.-26. (canceled)

27. A compound of formula II: 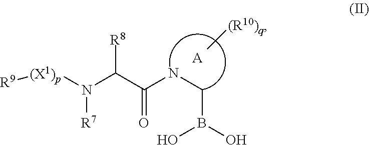
or a pharmaceutically acceptable salt thereof, wherein
A is a 4- to 7-membered heterocycle;
R⁷ is H or (C₁-C₆) alkyl;
R⁸ is H or (C₁-C₆) alkyl;
R¹⁰ is halogen, nitro, cyano, amino, amido, hydroxyl, alkoxy, aryloxy, acyloxy, carboxyl, thiol, alkylthio, arylthio, acylthio, alkyl, alkenyl, alkynyl, carbocyclyl, heterocyclyl, aryl, or heteroaryl;
q is an integer selected from 0 to 10, as valency permits;
X¹ is an alpha amino acid residue, or —X^{1A}—X^{1B}—X^{1C}—X^{1D}— wherein each X^{1A}, X^{1B}, X^{1C} and X^{1D} is independently a bond, —C(O)—, —CH₂C(O)—, alpha amino acid residue, substituted or unsubstituted (C₁-C₁₂) alkylene, substituted or unsubstituted 2 to 12 membered heteroalkylene, substituted or unsubstituted (C₃-C₈) cycloalkylene, substituted or unsubstituted 5 to 8 membered heterocycloalkylene, substituted or unsubstituted (C₆-C₈) arylene, or substituted or unsubstituted 5 to 8 membered heteroarylene, provided that at least one of X^{1A}, X^{1B}, X^{1C} and X^{1D} is not a bond;
p is an integer selected from 0 to 5; and
R⁹ is a moiety comprising at least one boron atom.

28. The compound of claim 27, or a pharmaceutically acceptable salt thereof, wherein the compound is a compound of formula II-a:

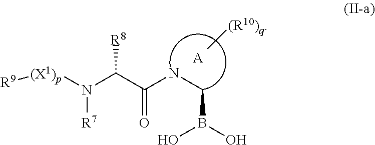

29. The compound of claim 27, or a pharmaceutically acceptable salt thereof, wherein the compound is a compound of formula II-b:

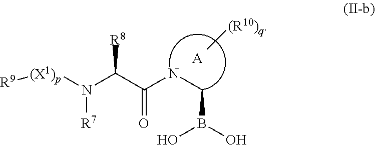

30. The compound of claim 27, or a pharmaceutically acceptable salt thereof, wherein A is a 5-membered heterocycle.

31. The compound of claim 27, or a pharmaceutically acceptable salt thereof, wherein R⁷ is H.

32. (canceled)

33. The compound of claim 27, or a pharmaceutically acceptable salt thereof, wherein R⁸ is methyl.

34. (canceled)

35. The compound of claim 27, or a pharmaceutically acceptable salt thereof, wherein X¹ comprises a natural or modified alpha amino acid residue.

36. The compound of claim 35, or a pharmaceutically acceptable salt thereof, wherein the natural alpha amino acid residue is selected from the group consisting of Val, Gly, Ile, Ala, Leu, Met, Phe, Tyr, and Trp.

37. (canceled)

38. The compound of claim 27, or a pharmaceutically acceptable salt thereof, wherein:
each X^{1A} and X^{1D} is independently —CH₂CH₂C(O)—, —CH₂C(O)— or —C(O)—; and
each X^{1B} and X^{1C} is independently a substituted or unsubstituted cyclohexylene, substituted or unsubstituted phenylene, or 2 to 6 membered heteroalkylene.

39. The compound of claim 27, or a pharmaceutically acceptable salt thereof, wherein:
X^{1A} is —CH₂CH₂C(O)—, —CH₂C(O)— or —C(O)—; and
X^{1B}, X^{1C} and X^{1D} is independently a bond or amino acid residue wherein at least one of X^{1B}, X^{1C} and X^{1D} is not a bond.

40. (canceled)

41. The compound of claim 27, or a pharmaceutically acceptable salt thereof, wherein R⁹ is a moiety comprising at least one ¹⁰B.

42. The compound of claim 27, or a pharmaceutically acceptable salt thereof, wherein R⁹ is a moiety comprising carborane.

43. The compound of claim 42, or a pharmaceutically acceptable salt thereof, wherein the carborane is decaborane.

44. The compound of claim 27, or a pharmaceutically acceptable salt thereof, wherein the boron atom of the boronic acid attached to A comprises a greater than natural abundance of ¹⁰B.

45. (canceled)

46. The compound of claim 27, or a pharmaceutically acceptable salt thereof, wherein the boron atom of the boronic acid attached to A comprises at least about 50% ¹⁰B.

47. A pharmaceutical composition, comprising a compound or composition of claim 27.

48. A method of treating cancer, comprising administering to a subject in need thereof an effective amount of a compound of claim 27.

49. (canceled)

50. (canceled)

51. An in vivo method of concentrating neutrons in a cell, comprising (i) administering to a subject an effective amount of a compound of claim 27; and (ii) irradiating the subject with neutrons.
